(19) United States
(12) Patent Application Publication
(10) Pub. No.: US 2023/0383270 A1
(43) Pub. Date: Nov. 30, 2023

(54) CRISPR/CAS-BASED BASE EDITING COMPOSITION FOR RESTORING DYSTROPHIN FUNCTION

(71) Applicant: Duke University, Durham, NC (US)

(72) Inventors: Charles A. Gersbach, Chapel Hill, NC (US); Veronica Gough, Durham, NC (US)

(21) Appl. No.: 18/031,313

(22) PCT Filed: Oct. 12, 2021
(86) PCT No.: PCT/US2021/054636

§ 371 Date: Apr. 11, 2023

Related U.S. Application Data
(60) Provisional application No. 63/183,545, filed on May 3, 2021.
(60) Provisional application No. 63/091,880, filed on Oct. 14, 2020.
(60) Provisional application No. 63/090,685, filed on Oct. 12, 2020.

(51) Int. Cl. C12N 9/22; C12N 9/78; A61P 21/00

(52) U.S. Cl. CPC C12N 9/22; C12N 9/78; A61P 21/00; C12N 2310/20; C12N 2320/33; C12Y 305/04001

(57) ABSTRACT

Disclosed herein are CRISPR/Cas-based base editing compositions and methods for treating Duchenne Muscular Dystrophy by restoring dystrophin function.

### CROSS-REFERENCE TO RELATED APPLICATIONS

This application claims priority to U.S. Provisional Patent Application No. 63/090,685 filed Oct. 12, 2020, U.S. Provisional Patent Application No. 63/091,880 filed Oct. 14, 2020, and U.S. Provisional Patent Application No. 63/183,545 filed May 3, 2021, each of which is incorporated herein by reference in its entirety.

### STATEMENT REGARDING FEDERALLY SPONSORED RESEARCH

This invention was made with government support under contract number R01AR069085 awarded by the National Institutes of Health. The government has certain rights in the invention.

### FIELD

The present disclosure is directed to CRISPR/Cas-based base editing compositions and methods for treating Duchenne Muscular Dystrophy by restoring dystrophin function.

### INTRODUCTION

Duchenne muscular dystrophy (DMD) is typically caused by deletions of one or more exons from the dystrophin gene, leading to disruption of the reading frame. Expression of dystrophin protein can be restored by correcting the reading frame by inducing the exclusion of one or more additional exons. The removal of introns and inclusion of selected exons during mRNA splicing is critical to normal gene function and is often misregulated in genetic disorders. Technologies that modulate mRNA processing and exon selection, such as exon skipping approaches, may be used to study and treat these diseases. Exon skipping aims to restore the correct reading frame or induce alternative splicing by blocking the recognition of splicing sequences by the spliceosome, leading to removal of specific exons along with the adjacent introns. Studies have shown that by targeting Cas9 to the splice acceptor of exons, the indels produced during DNA repair can disrupt the splice site and induce exclusion of the exon. However, there remains a need for the ability to precisely alter the splice sites in the dystrophin gene in order to restore fully and/or partially dystrophin function.

### SUMMARY

In an aspect, the disclosure relates to a CRISPR/Cas-based base editing system for altering an RNA splice site encoded in the genomic DNA of a subject. The CRISPR/Cas-based base editing system may include a fusion protein and at least one guide RNA (gRNA), wherein the fusion protein comprises a Cas protein and a base-editing domain, and wherein the at least one gRNA targets a sequence comprising at least one of SEQ ID NOs: 21-23 or 43 or a complement or a fragment thereof and/or the gRNA comprises a sequence selected from SEQ ID NOs: 24-26 or 44 or a complement or a fragment thereof.

In a further aspect, the disclosure relates to a CRISPR/Cas-based base editing system for altering an RNA splice site encoded in the genomic DNA of a subject. The CRISPR/Cas-based base editing system may include a fusion protein and at least one guide RNA (gRNA), wherein the fusion protein comprises a Cas protein and a base-editing domain, and wherein the base-editing domain comprises a polypeptide selected from SEQ ID NOs: 45-52 and/or is encoded by a polynucleotide comprising a sequence selected from SEQ ID NOs: 53-60.

In some embodiments, the fusion protein comprises a polypeptide selected from SEQ ID NOs: 27-34 and/or is encoded by a polynucleotide comprising a sequence selected from SEQ ID NOs: 35-42. In some embodiments, altering the RNA splice site encoded in the genomic DNA results in exclusion or inclusion of at least one exon sequence in an RNA transcript.

Another aspect of the disclosure provides a CRISPR/Cas-based base editing system for restoring dystrophin function in a subject. The CRISPR/Cas-based base editing system may include a fusion protein and at least one guide RNA (gRNA), wherein the fusion protein comprises a Cas protein and a base-editing domain, wherein the at least one gRNA targets a sequence comprising at least one of SEQ ID NOs: 21-23 or 43 or a complement or a fragment thereof and/or the gRNA comprises a sequence selected from SEQ ID NOs: 24-26 or 44 or a complement or a fragment thereof.

Another aspect of the disclosure provides a CRISPR/Cas-based base editing system for restoring dystrophin function in a subject. The CRISPR/Cas-based base editing system may include a fusion protein and at least one guide RNA (gRNA), wherein the fusion protein comprises a Cas protein and a base-editing domain, and wherein base-editing domain comprises a polypeptide selected from SEQ ID NOs: 45-52 and/or is encoded by a polynucleotide comprising a sequence selected from SEQ ID NOs: 53-60.

In some embodiments, the fusion protein comprises a polypeptide selected from SEQ ID NOs: 27-34 and/or is encoded by a polynucleotide comprising a sequence selected from SEQ ID NOs: 35-42. In some embodiments, the subject has a mutated dystrophin gene, and wherein the at least one guide RNA (gRNA) targets an RNA splice site in the mutated dystrophin gene of the subject. In some embodiments, administration of the CRISPR/Cas-based base editing system to the subject results in at least one exon sequence being excluded or included in an RNA transcript of the dystrophin gene of the subject and the reading frame of dystrophin gene in the subject being restored. In some embodiments, the Cas protein comprises a Cas9, and wherein the Cas9 comprises at least one amino acid mutation which eliminates the nuclease activity of Cas9. In some embodiments, the at least one amino acid mutation is at least one of D10A, H840A, or a combination thereof, in the amino acid sequence corresponding to SEQ ID NO: 2 or 3. In some embodiments, the Cas protein is a Streptococcus pyogenes Cas9 protein or a Staphylococcus aureus Cas9 protein. In some embodiments, the Cas protein comprises an amino acid sequence of SEQ ID NO: 4 or 5. In some embodiments, the base-editing domain further comprises (i) a cytidine deaminase domain and (ii) at least one uracil glycosylase inhibitor (UGI) domain. In some embodiments, the cytidine deaminase domain comprises an apolipoprotein B mRNA-editing enzyme, catalytic polypeptide-like (APOBEC) deaminase. In some embodiments, the cytidine deaminase domain comprises an APOBEC 1 deaminase. In some embodiments, the cytidine deaminase domain comprises a rat APOBEC 1 deaminase. In some embodiments, the at least one UGI domain comprises a domain capable of inhibiting UDG activity. In some embodiments, the at least one UGI domain comprises the amino acid sequence of SEQ ID NO: 20 or an amino acid sequence encoded by the polynucleotide sequence of SEQ ID NO: 6 or SEQ ID NO: 18. In some embodiments, the base-editing domain comprises one UGI domain or two UGI domains. In some embodiments, the fusion protein comprises the structure: NH₂[ABE]-[Cas protein]-COOH, and wherein each instance of “-” comprises an optional linker. In some embodiments, the fusion protein comprises the structure: NH₂-[Cas protein]-[ABE]-COOH, and wherein each instance of “-” comprises an optional linker. In some embodiments, the fusion protein further comprises a nuclear localization sequence (NLS).

Another aspect of the disclosure provides an isolated polynucleotide encoding a CRISPR/Cas-based base editing system as detailed herein. In some embodiments, the polynucleotide comprises a first polynucleotide encoding the fusion protein and a second polynucleotide encoding the gRNA. Another aspect of the disclosure provides a vector comprising the isolated polynucleotide. In some embodiments, the vector comprises a heterologous promoter driving expression of the isolated polynucleotide. Another aspect of the disclosure provides a cell comprising the isolated polynucleotide.

Another aspect of the disclosure provides a composition for restoring dystrophin function in a cell having a mutant dystrophin gene, the composition comprising a CRISPR/Cas-based base editing system as detailed herein.

Another aspect of the disclosure provides a kit comprising a CRISPR/Cas-based base editing system of as detailed herein, an isolated polynucleotide as detailed herein, a vector as detailed herein, a cell as detailed herein, or a composition as detailed herein.

Another aspect of the disclosure provides a method for restoring dystrophin function in a cell or a subject having a mutant dystrophin gene. The method may include contacting the cell or the subject with a CRISPR/Cas-based base editing system as detailed herein. In some embodiments, an “AG” splice acceptor in exon 45 of the mutant dystrophin gene is converted to an “GG” sequence and the dystrophin function is restored by exon 45 skipping. In some embodiments, the subject is suffering from Duchenne Muscular Dystrophy.

The disclosure provides for other aspects and embodiments that will be apparent in light of the following detailed description and accompanying figures.

### BRIEF DESCRIPTION OF THE DRAWINGS

FIGS. 1A-ID. FIG. 1A shows a CRISPR/Cas9-based base editor design (Komor et al., Nature 2016, 533, 420-424) in which the Cas9 component can be derived from various species, such as Streptococcus pyogenes and Staphylococcus aureus. In some embodiments, the base editor design comprises a cytidine deaminase, a linker, a nCas9, and an uracil glycosylase inhibitor (UGI). The uracil DNA glycosylase catalyzes reversion of U:G→C:G. In some embodiments, the base editor design comprises a cytidine deaminase, such as a rat cytidine deaminase, e.g., rAPOBEC1. In some embodiments, the base editor design comprises a XTEN linker (16 aa). In some embodiments, the base editor design comprises a nCas9 (RNA-guided and promotes mismatch repair on the strand with the unedited G). In some embodiments, the base editor design comprises a UGI, such as a UGI from Bacillus subtilis bacteriophage PBS1. FIG. 1B shows an alternative CRISPR/Cas9-based base editor design (Koblan et al. Nature Biotech. 2018, 36, 843-846). In the BE4max design, bipartite nuclear localization signals were further added to the N and C termini. 8 codon usages were tested. In the AncBE4max design, an ancestral sequence reconstruction on APOBEC was used. In some embodiments, the Cas9 component can be derived from various species, such as Streptococcus pyogenes and Staphylococcus aureus. FIG. 1C shows the base edit of C→T (or G→A) in a 5 bp window of positions 4-8 of protospacer. FIG. 1D shows the mechanism of base excision repair.

FIGS. 2A-2B. FIG. 2A shows a schematic showing R-loop formation by the base editors and the interaction between the cytidine deaminase enzyme and ssDNA. FIG. 2B shows a schematic for designing gRNAs to base edit splice acceptors and the strict requirement for “AG” splice acceptor to fall within the editing window determined by the availability of a PAM (which changes depending on species of Cas9—“Sp” is Streptococcus pyogenes and ‘Sa’ is Staphylococcus aureus).

FIGS. 3A-3C. FIG. 3A shows the splice acceptor design strategy for exons 44 and 45 (as well as many others) in which gi and G2 are targeted for base editing. FIG. 3B shows the % G>A base editing at the Exon 44 splice acceptor site (N=3) using an exon 44 gRNA of 5′-CGCCTGCAGGTAAAAGCATA-3′ (SEQ ID NO: 9). FIG. 3C shows the % G>A base editing at the Exon 45 splice acceptor site (N=3) using an exon 45 gRNA corresponding to 5′-GTTCCTGTAAGATACCAAAA-3′ (SEQ ID NO: 1).

FIGS. 4A-4D. FIG. 4A shows a schematic of exons 41-50 of the dystrophin gene. FIG. 4B shows the expected sequence of a dystrophin gene which would result from deletion of exon 44. As a result, intron 43 would transition directly into intron 44. FIG. 4C shows the sequence of a dystrophin gene in which exon 44 was deleted. Insertions or deletions may be present at the junction intron 43 and intron 44 following deletion of exon 44. FIG. 4D shows confirmation of the deletion of exon 44 of the dystrophin gene in clone c11 compared to clone c2 without a deletion in exon 44.

FIG. 5 shows a schematic of myogenic differentiation of iPSCs.

FIG. 6 shows myogenic differentiation of iPSCs in which the A44 mutation ablates the dystrophin protein.

FIG. 7 shows an outline for A44 iPSC editing.

FIGS. 8A-8B. FIG. 8A shows the % G>A base editing events in the A44 iPSC using BE4max. FIG. 8B shows all gVG03 d12 editing events in the A44 iPSC using BE4max.

FIGS. 9A-9B. FIG. 9A shows the % G>A base editing events in the A44 iPSC using AncBE4max. FIG. 9B shows all gVG03 d12 editing events in the A44 iPSC using AncBE4max.

FIG. 10 shows A44 iPSC editing after 12 days using BE4max and AncBE4max.

FIG. 11 shows RT-PCR of MyoD differentiation of edited cells.

FIG. 12 shows % Non-G base editing events in the A44 iPSC using AncBE4max delivered by lentivrus on day 7 (D7) and day 14 (D14).

FIG. 13 shows % Non-G base editing events in the A44 iPSC using AncBE4max delivered by electroporation on day 7 (D7) and day 14 (D14).

FIG. 14 shows a schematic diagram of the wild-type (NT), A44, and A44-45 versions of the dystrophin gene (left), and a Western blot of MyoD differentiated A44 iPSC cells edited with AncBE4max and exon 45 gRNA (right).

FIGS. 15A-15C. FIG. 15A is a schematic diagram of four adenine base editors (ABEs) used (see Example 2). FIG. 15B shows A3, the splice acceptor target that was edited for exon skipping. FIG. 15C shows results of a transfection experiment performed in HEK293T cells. ABE8e with gVG56 enabled conversion of 38.6% of the splice acceptor A3s to a non-A base, with G being the predominant edit.

FIG. 16 shows results of a transfection experiment performed in HEK293T cells with an expanded panel of four additional ABE variants, with the same three gRNAs tested with each editor. Across all variants tested, the gRNA gVG56 showed the greatest ability to edit the exon 45 splice acceptor (A3) compared to gVG55 and gVG56.

FIGS. 17A-17G. FIG. 17A is a schematic diagram of the gRNA design to edit the “A” of the hDMD exon 45 splice acceptor with SpCas9-based ABEs. FIG. 17B is a graph showing exon 45 splice acceptor base editing (adenine A3 conversion to C, G, or T) with a panel of ABEs with g01, g02, or g03 gRNAs in HEK293T cells (n=3, error bars represent SEM). Any edit away from “A” should disrupt the “AG” splice acceptor. ABE8e and ABE8.17, when paired with g02, showed the most efficient editing at this position. FIG. 17C is a schematic diagram of the gRNA design to edit the “G” of the hDMD exon 45 splice acceptor with SpCas9-based ABEs. FIG. 17D is a graph showing exon 45 splice acceptor base editing (guanine G1 conversion to C, A, or T) with a panel of ABEs with g04 gRNA in HEK293T cells (n=3, error bars represent SEM). FIG. 17E and FIG. 17F are graphs showing bystander editing of neighboring As with ABE8e (FIG. 17E) and ABE8.17m (FIG. 17F). Bystander edits are not expected to interfere with slice site disruption or coding sequence. FIG. 17G is a graph showing the purity of ABE8e and ABE8.17m products with g02.

FIGS. 18A-18C. FIG. 18A is a schematic diagram for the creation of a A44 human iPSC line. SpCas9 and two gRNAs were used to excise exon 44, which shifts dystrophin out-of-frame. The reading frame in Δ44 cells can be restored by skipping exon 45. FIG. 18B is a schematic diagram showing lentiviral constructs for iPSC editing and differentiation. Δ44 iPSCs were transduced with either ABE8e or ABE8.17m and selected to create stable lines. At day 0, either g02 or a scrambled control were transduced, but not selected on. To achieve dystrophin expression. ABE+gRNA cells were cultured in skeletal muscle media (SMM), transduced with a lentiviral construct with constitutive MyoD cDNA, and further differentiated in low serum conditions. FIG. 18C is a graph showing that ABE8e+g02 exhibited 88.6% splice acceptor base editing in Δ44 iPSCs 4 days post-gRNA transduction (no selection on gRNA lenti). Minimal increases in DNA editing were observed during the MyoD differentiation.

FIGS. 19A-19C. FIG. 19A is a gel showing RT-PCR products on cDNA from Day 28 of the Δ44 iPSCs+ABE+gRNA+MyoD differentiation. The high level of exon 45 splice acceptor base editing observed with ABE8e+g02 corresponds with a strong shift towards transcripts skipping exon 45. FIG. 19B is a graph showing the quantification of the Day 28 cDNA exon skipping by ddPCR. ABE8e+g02 exhibited 96.6% exon 45 skipping. FIG. 19C is a Westem blot showing restoration of dystrophin protein expression with splice acceptor base editing. ABE8e+g02 rescued dystrophin protein expression that was not present in unedited Δ44 iPSCs.

FIG. 20 is a schematic diagram of canonical splice sites delineating intron-exon boundaries. Both adenine and cytosine base editors can be used to disrupt the splice acceptor and force exon skipping.

FIGS. 21A-21E. FIG. 21A is a schematic diagram of the reading frame of hDMD exons 43-46. The deletion of exon 44 disrupts the reading frame, which can be rescued by editing of the exon 45 splice acceptor and subsequent exon 45 skipping. To accomplish this editing in iPSC-derived cardiomyocytes (CM), ABE8e and ABE8.17m were delivered in lentiviral constructs. FIG. 21B is a graph showing base editing in Δ44 iPSC-derived CMs 5 days after transduction of base editor and gRNA lentiviruses without selection. All adenines in the editing window are represented, with the main splice acceptor target at A3. The percent of reads with conversion of A to C, G, or T are plotted, along with the percent of reads containing indels (black) (n=3, error bars represent SEM). FIG. 21C is a gel showing the products from endpoint RT-PCR on RNA from base edited CMs amplified with primers in exons 42 and 46. FIG. 21D is a graph showing ddPCR quantification of exon skipping in base edited CMs. The editing frequency was calculated as edited transcripts divided by the sum of edited and unedited transcripts (n=3, error bars represent SEM). FIG. 21E is a Westem blot for base edited CMs, stained for dystrophin (MANDYS108) and GAPDH.

### DETAILED DESCRIPTION

The present disclosure provides CRISPR/Cas-based base editing compositions and methods for treating Duchenne Muscular Dystrophy (DMD) by restoring dystrophin function. DMD is typically caused by deletions in the dystrophin gene that disrupt the reading frame. Many strategies to treat DMD aim to restore the reading frame by removing or skipping over an additional exon, as it has been shown that internally truncated dystrophin protein can still be partially functional. There are conserved sequences that mark the boundaries between introns and exons in mammalian genes. One important splice site is the “AG” that precedes exons and is called the splice acceptor. Full nuclease Cas9 has been used to target the splice acceptors of dystrophin exons to force skipping, thereby relying on the semi-random indels formed during the DNA repair process to ablate the splice site. The presently disclosed CRISPR/Cas-based base editing system allows for a more precise base editing method to reliably convert the “AG” splice acceptor to an “AA” or “GG” that will promote exon skipping. In contrast to the semi-random indels generated by the conventional CRISPR-Cas9 system, base editing technologies have been developed for the precise modification of a single base pair without inducing double-stranded DNA breaks. Base editors can change a C directly to a T, or a G to A on the reverse strand, and they may be targeted to both splice donors “GT” and acceptors “AG” of a variety of exons to modulate mRNA splicing.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value. The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the CRISPR/Cas-based base editing system.

“Chromatin” as used herein refers to an organized complex of chromosomal DNA associated with histones.

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a polynucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, for example, to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a construct or system as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 5000 live male births. DMD is the result of inherited or spontaneous mutations that cause nonsense or frame shift mutations in the dystrophin gene. The majority of dystrophin mutations that cause DMD are deletions of exons that disrupt the reading frame and cause premature translation termination in the dystrophin gene. DMD patients typically lose the ability to physically support themselves during childhood, become progressively weaker during the teenage years, and die in their twenties.

“Dystrophin” as used herein refers to a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane that is responsible for regulating muscle cell integrity and function. The dystrophin gene or “DMD gene” as used interchangeably herein is 2.2 megabases at locus Xp21. The primary transcription measures about 2,400 kb with the mature mRNA being about 14 kb. 79 exons code for the protein which is over 3500 amino acids.

“Exon 45” as used herein refers to the 45 exon of the dystrophin gene. Exon 45 is frequently adjacent to frame-disrupting deletions in DMD patients and has been targeted in clinical trials for oligonucleotide-based exon skipping.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or“frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” as used herein refers to changing a mutant gene that encodes a dysfunctional protein or truncated protein or no protein at all, such that a full-length functional or partially full-length functional protein expression is obtained. Genome editing may include correcting or restoring a mutant gene. Genome editing may include base editing for altering a splice acceptor site or splice donor sequence. Genome editing, for example base editing, may be used to treat disease or enhance muscle repair by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (e.g., a “fusion protein,” where the two subsequences are encoded by a single nucleic acid sequence).

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA. RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine, and isoguanine. Nucleic acids may be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be, for example, 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and the CMV IE promoter. Promoters that target muscle-specific stem cells may include the CK8 promoter, the Spc5-12 promoter, and the MHCK7 promoter.

The term “recombinant” when used with reference, for example, to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Skeletal muscle” as used herein refers to a type of striated muscle, which is under the control of the somatic nervous system and attached to bones by bundles of collagen fibers known as tendons. Skeletal muscle is made up of individual components known as myocytes, or “muscle cells,” sometimes colloquially called “muscle fibers.” Myocytes are formed from the fusion of developmental myoblasts (a type of embryonic progenitor cell that gives rise to a muscle cell) in a process known as myogenesis. These long, cylindrical, multinucleated cells are also called myofibers.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Skeletal muscle condition” as used herein refers to a condition related to the skeletal muscle, such as muscular dystrophies, aging, muscle degeneration, wound healing, and muscle weakness or atrophy.

“Subject” and “patient” as used herein interchangeably refers to any vertebrate, including, but not limited to, a mammal. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject or patient may be undergoing other forms of treatment. The subject may be of any age or stage of development, such as, for example, an adult, an adolescent, a child, such as age 0-2, 2-4, 2-6, or 6-12 years, or an infant, or an infant, such as age 0-1 years. The subject may be male. The subject may be female. In some embodiments, the subject has a specific genetic marker.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is the dystrophin gene. “Target region” as used herein refers to the region of the target gene to which the CRISPR/Cas9-based gene editing or targeting system is designed to bind.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treat,” “treating,” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of an antibody or pharmaceutical composition of the present invention to a subject that is not at the time of administration afflicted with the disease. “Preventing” also refers to preventing the recurrence of a disease or of one or more symptoms associated with such disease. “Treatment” and “therapeutically” refer to the act of treating, as “treating” is defined above.

“Variant” used herein with respect to a nucleic acid means (i) a portion or fragment of a referenced polynucleotide sequence; (ii) the complement of a referenced polynucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, for example, replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode the CRISPR/Cas-based base editing system described herein, including a polynucleotide sequence encoding the fusion protein, such as SEQ ID NO: 7 or SEQ ID NO: 8, and/or at least one gRNA polynucleotide sequence of SEQ ID NO: 1 or one of SEQ ID NOs: 21-26 or 43-44.

### 2. CRISPR/CAS-BASED BASE EDITING SYSTEM FOR RESTORING DYSTROPHIN

Provided herein are CRISPR/Cas-based base editing systems. The CRISPR/Cas-based base editing systems may be used for altering an RNA splice site encoded in the genomic DNA of a subject. The CRISPR/Cas-based base editing systems may be for use in restoring dystrophin gene function. The CRISPR/Cas-based base editing system may include a fusion protein and at least one guide RNA (gRNA). In some embodiments, the at least one gRNA targets a sequence comprising at least one of SEQ ID NOs: 21-23 or 43 or a complement or a variant or a fragment thereof, and/or the at least one gRNA comprises a sequence selected from SEQ ID NOs: 24-26 or 44 or a complement or a variant or a fragment thereof. In some embodiments, the at least one gRNA binds and targets a polynucleotide sequence corresponding to SEQ ID NO: 1. In some embodiments, the at least one gRNA is encoded by the polynucleotide sequence of SEQ ID NO: 1. The fusion protein can comprise two heterologous polypeptide domains. In some embodiments, the fusion protein comprises a Cas protein and a base-editing domain. In some embodiments, the base-editing domain comprises an adenine base editor (ABE). In some embodiments, the fusion protein comprises a polypeptide selected from SEQ ID NOs: 27-34 and/or is encoded by a polynucleotide comprising a sequence selected from SEQ ID NOs: 35-42. In some embodiments, the at least one gRNA binds and targets a polynucleotide sequence corresponding to: a) a fragment of SEQ ID NO: 1; b) a complement of SEQ ID NO: 1, or fragment thereof; c) a nucleic acid that is substantially identical to SEQ ID NO: 1, or complement thereof; or d) a nucleic acid that hybridizes under stringent conditions to SEQ ID NO: 1, complement thereof, or a sequence substantially identical thereto. In some embodiments, the at least one gRNA comprises a polynucleotide sequence corresponding to SEQ ID NO: 1, or variant thereof.

a. Dystrophin Gene

Dystrophin is a rod-shaped cytoplasmic protein which is a part of a protein complex that connects the cytoskeleton of a muscle fiber to the surrounding extracellular matrix through the cell membrane. Dystrophin provides structural stability to the dystroglycan complex of the cell membrane. The dystrophin gene is 2.2 megabases at locus Xp21. The primary transcription measures about 2,400 kb with the mature mRNA being about 14 kb. 79 exons code for the protein which is over 3500 amino acids. Normal skeleton muscle tissue contains only small amounts of dystrophin but its absence of abnormal expression leads to the development of severe and incurable symptoms. Some mutations in the dystrophin gene lead to the production of defective dystrophin and severe dystrophic phenotype in affected patients. Some mutations in the dystrophin gene lead to partially-functional dystrophin protein and a much milder dystrophic phenotype in affected patients.

DMD is the result of inherited or spontaneous mutations that cause nonsense or frame shift mutations in the dystrophin gene. Naturally occurring mutations and their consequences are relatively well understood for DMD. It is known that in-frame deletions that occur in the exon 45-55 regions contained within the rod domain can produce highly functional dystrophin proteins, and many carriers are asymptomatic or display mild symptoms. Furthermore, more than 60% of patients may theoretically be treated by targeting exons in this region of the dystrophin gene. Efforts have been made to restore the disrupted dystrophin reading frame in DMD patients by skipping non-essential exon(s) (for example, exon 45 skipping) during mRNA splicing to produce internally deleted but functional dystrophin proteins. The deletion of internal dystrophin exon(s) (for example, deletion of exon 45) retains the proper reading frame and can generate an internally truncated but partially functional dystrophin protein. Deletions between exons 45-55 of dystrophin result in a phenotype that is much milder compared to DMD.

Human DMD exon 45 may be an attractive exon for demonstrating the application of base editing to DMD exon skipping because it is the exon that may treat the second largest group of DMD patients when skipped (8.1%). In certain embodiments, excision of exon 45 to restore reading frame ameliorates the phenotype in DMD subjects, including DMD subjects with deletion mutations. In certain embodiments, exon 45 of a dystrophin gene refers to the 45th exon of the dystrophin gene. Exon 45 is frequently adjacent to frame-disrupting deletions in DMD patients and has been targeted in clinical trials for oligonucleotide-based exon skipping.

The CRISPR/Cas-based base editing systems as detailed herein may be used for altering an RNA splice site encoded in the genomic DNA of a subject. In some embodiments, altering the RNA splice site encoded in the genomic DNA results in exclusion or inclusion of at least one exon sequence in an RNA transcript. The CRISPR/Cas-based base editing systems as detailed herein may be used for restoring dystrophin function in a subject. In some embodiments, the subject has a mutated dystrophin gene, and at least one guide RNA (gRNA) targets an RNA splice site in the mutated dystrophin gene of the subject. In some embodiments, administration of the CRISPR/Cas-based base editing system to the subject results in at least one exon sequence being excluded or included in an RNA transcript of the dystrophin gene of the subject, and the reading frame of dystrophin gene in the subject being restored.

The presently disclosed systems and vectors can alter a splice acceptor site at exon 45 in the dystrophin gene, e.g., the human dystrophin gene. Altering of the splice acceptor site can result in exon 45 being deleted from the dystrophin protein product (i.e., exon 45 skipping) and can increase the function or activity of the encoded dystrophin protein, or results in an improvement in the disease state of the subject. In certain embodiments, exon 45 skipping can restore the dystrophin reading frame. In some embodiments, the splice acceptor site at exon 45 is within a sequence comprising the polynucleotide sequence of SEQ ID NO: 1. In some embodiments, the splice acceptor site at exon 45 is within a sequence comprising the polynucleotide sequence selected from SEQ ID NOs: 21-23 and 43.

A presently disclosed system or genetic construct (e.g., a vector) can mediate highly efficient exon 45 skipping of a dystrophin gene (for example, the human dystrophin gene). A presently disclosed system or genetic construct (for example, a vector) may restore dystrophin protein expression in cells from DMD patients. Exon 45 is frequently adjacent to frame-disrupting deletions in DMD. Elimination of exon 45 from the dystrophin transcript by exon skipping can be used to treat approximately 8% of all DMD patients. A presently disclosed system or genetic construct (for example, a vector) may be transfected into human DMD cells and mediate efficient gene modification and conversion to the correct reading frame. Protein restoration may be concomitant with frame restoration and detected in a bulk population of CRISPR/Cas-based base editing system-treated cells.

b. Fusion Protein

The CRISPR/Cas-based base editing system includes a fusion protein or a nucleic acid sequence encoding a fusion protein. The fusion protein comprises a Cas protein and a base-editing domain. In some embodiments, the nucleic acid sequence encoding the fusion protein is DNA. In some embodiments, the nucleic acid sequence encoding the fusion protein is RNA.

i) Cas Protein

The Cas protein forms a complex with the 3′ end of a gRNA. The specificity of the CRISPR-based system depends on two factors: the targeting sequence and the protospacer-adjacent motif (PAM). The targeting or recognition sequence is located on the 5′ end of the gRNA and is designed to pair with base pairs on the host DNA (target nucleic acid or target DNA) at the correct DNA sequence known as the protospacer. By simply exchanging the recognition sequence of the gRNA, the Cas protein can be directed to new genomic targets. The PAM sequence is located on the DNA to be altered and is recognized by a Cas protein. PAM recognition sequences of the Cas protein can be species specific.

In some embodiments, the CRISPR/Cas-based base editing system may include a Cas9 protein, such as a catalytically dead dCas9. Cas9 protein is an endonuclease that cleaves nucleic acid and is encoded by the CRISPR loci and is involved in the Type II CRISPR system. A Cas9 molecule can interact with one or more gRNA molecule and, in concert with the gRNA molecule(s), localizes to a site which comprises a target domain, and in certain embodiments, a PAM sequence. The ability of a Cas9 molecule to recognize a PAM sequence can be determined, for example, using a transformation assay as described previously (Jinek 2012). In some embodiments, the Cas9 protein is from Streptococcus pyogenes. In some embodiments, the Cas9 protein comprises the polypeptide sequence of SEQ ID NO: 2. In some embodiments, the Cas9 protein is from Staphylococcus aureus. In some embodiments, the Cas9 protein comprises the polypeptide sequence of SEQ ID NO: 3.

In some embodiments, the Cas9 protein may be mutated so that the nuclease activity is reduced or inactivated. An inactivated Cas9 protein (“iCas9”, also referred to as “dCas9”) with no endonuclease activity may be targeted to genes in bacteria, yeast, and human cells by gRNAs to silence gene expression through steric hindrance. Exemplary mutations with reference to the S. pyogenes Cas9 sequence to reduce or inactivate nuclease activity include: D10A, E762A, H840A, N854A, N863A and/or D986A. Exemplary mutations with reference to the S. aureus Cas9 sequence to inactivate nuclease activity include D10A and N580A. In some embodiments, an inactivated Cas9 protein from Streptococcus pyogenes (iCas9, also referred to as “dCas9”; SEQ ID NO: 5) may be used. As used herein, “iCas9” and “dCas9” both may refer to a Cas9 protein that has the amino acid substitutions D10A and H840A and has its nuclease activity inactivated. In some embodiments, the Cas protein can be a mutant Cas9 protein that has the amino acid substitutions D10A (referred to as “nCas9” and has nickase activity; e.g., SEQ ID NO: 4).

The Cas9 protein or mutant Cas9 protein may be from any bacterial or archaea species, such as Streptococcus pyogenes, Staphylococcus aureus, Streptococcus thermophiles, or Neisseria meningitides. In some embodiments, the Cas protein or mutant Cas9 protein is a Cas9 protein derived from a bacterial genus of Streptococcus, Staphylococcus, Brevibacillus, Corynebacter, Sutterella, Legionella, Francisella, Treponema, Filifactor, Eubacterium, Lactobacillus, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter. In some embodiments, the Cas9 protein or mutant Cas9 protein is selected from the group, including, but not limited to, Streptococcus pyogenes, Francisella novicida, Staphylococcus aureus, Neisseria meningitides, Streptococcus thermophiles, Treponema denticola, Brevibacillus laterosporus, Campylobacter jejuni, Corynebactenum diphtheria, Eubacterium ventriosum, Streptococcus pasteurianus, Lactobacillus farciminis, Sphaerochaeta globus, Azospirillum, Gluconacetobacter diazotrophicus, Neisseria cinerea, Roseburia intestinalis, Parvibaculum lavamentivorans, Nitratifractor salsuginis, and Campylobacter lari.

In certain embodiments, the ability of a Cas9 molecule or mutant Cas9 protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (e.g., PAM sequences). In certain embodiments, a Cas9 molecule of S. pyogenes recognizes the sequence motif NGG (SEQ ID NO: 10) and directs cleavage of a target nucleic acid sequence 1 to 10, such as 3 to 5, bp upstream from that sequence (see, for example, Mali 2013). In certain embodiments, a Cas9 molecule of S. aureus recognizes the sequence motif NNGRR (R=A or G) (SEQ ID NO: 12) and directs cleavage of a target nucleic acid sequence 1 to 10, such as 3 to 5, bp upstream from that sequence. In certain embodiments, a Cas9 molecule of S. aureus recognizes the sequence motif NNGRRN (R=A or G) (SEQ ID NO: 13) and directs cleavage of a target nucleic acid sequence 1 to 10, such as 3 to 5, bp upstream from that sequence. In certain embodiments, a Cas9 molecule of S. aureus recognizes the sequence motif NNGRRT (R=A or G) (SEQ ID NO: 14) and directs cleavage of a target nucleic acid sequence 1 to 10, such as 3 to 5, bp upstream from that sequence. In certain embodiments, a Cas9 molecule of S. aureus recognizes the sequence motif NNGRRV (R=A or G; V=A or C or G) (SEQ ID NO: 15) and directs cleavage of a target nucleic acid sequence 1 to 10, such as 3 to 5, bp upstream from that sequence. In the aforementioned embodiments, N can be any nucleotide residue, e.g., any of A, G, C, or T. Cas9 molecules can be engineered to alter the PAM specificity of the Cas9 molecule.

In some embodiments, the Cas9 protein or mutant Cas9 protein can recognize a PAM sequence NGG (SEQ ID NO: 10) or NGA (SEQ ID NO: 19). In some embodiments, the Cas9 protein or mutant Cas9 protein can recognize a PAM sequence NNNRRT (SEQ ID NO: 11). In some embodiments, the Cas9 protein or mutant Cas9 protein is a Cas9 protein of S. aureus and recognizes the sequence motif NNGRR (R=A or G) (SEQ ID NO: 12), NNGRRN (R=A or G) (SEQ ID NO: 13), NNGRRT (R=A or G) (SEQ ID NO: 14), or NNGRRV (R=A or G) (SEQ ID NO: 15). In the aforementioned embodiments, N can be any nucleotide residue, e.g., any of A, G, C, or T. Cas9 molecules can be engineered to alter the PAM specificity of the Cas9 molecule.

Additionally or alternatively, a nucleic acid encoding a Cas9 molecule or Cas9 polypeptide may comprise a nuclear localization sequence (NLS). Nuclear localization sequences are known in the art. In some embodiments, the NLS comprises an amino acid sequence selected from SEQ ID NOs: 65-68, encoded by a polynucleotide sequence of SEQ ID NOs: 69-72, respectively.

ii) Base-Editing Domain

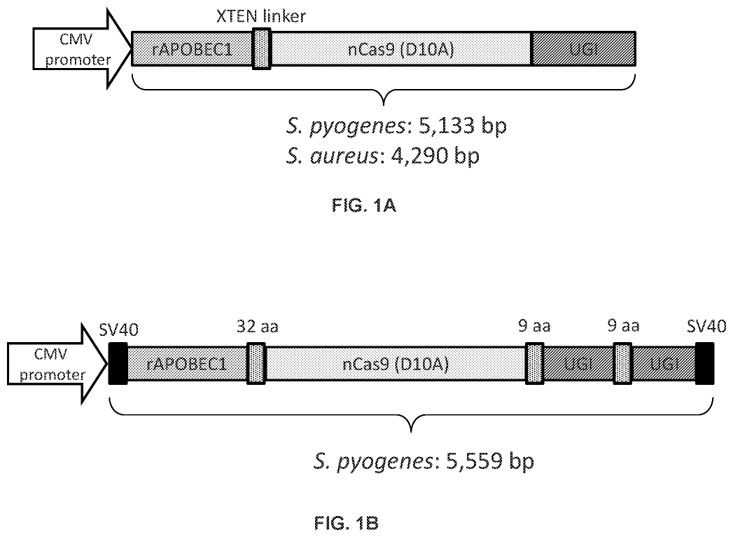

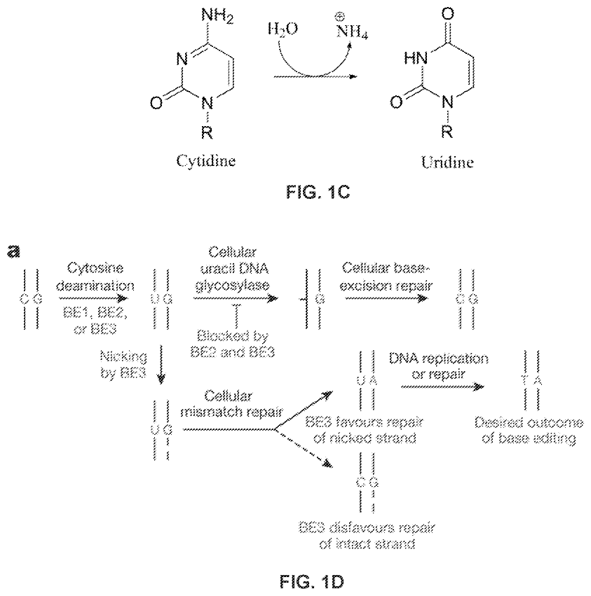

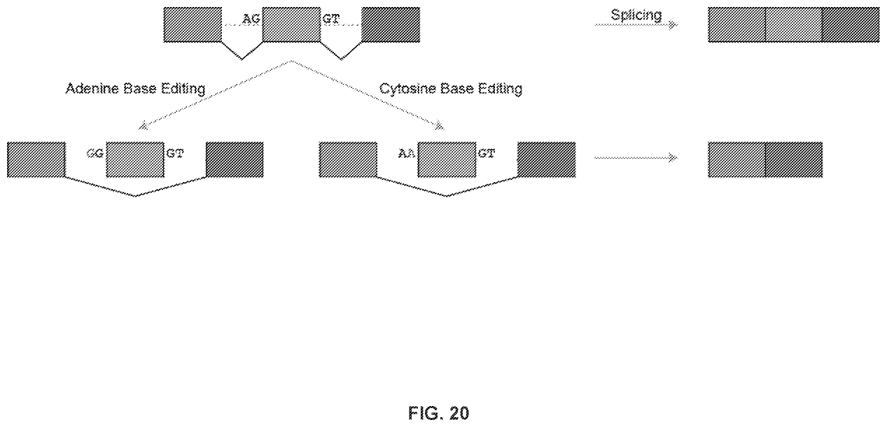

The fusion protein comprises a Cas protein and a base-editing domain. Base editing enables the direct, irreversible conversion of a specific DNA base into another base at a targeted genomic locus without requiring double-stranded DNA breaks (DSB). FIG. 1D shows one design process of the base editor. A base editing domain has sequence requirements for activity. In a 20 nucleotide protospacer, the target base may be within 4-8 nucleotides from the PAM-distal end. An exemplary splice acceptor is an “AG” immediately before the exon, and an exemplary splice donor is a “GT” immediately following the exon. Cas9 molecules from different species may use different PAMs, and thereby provide some flexibility in selecting the base to edit. Disruption of canonical splice sites can lead to exon skipping or activation of cryptic splice sites. Both adenine and cytosine base editors may be capable of disrupting an “AG” splice acceptor, converting it to either a “GG” or “AA”, respectively (FIG. 20). In some embodiments, an “AG” splice acceptor in exon 45 of the mutant dystrophin gene is converted to an “GG” sequence by a base editing domain, such as an adenine base editor, and the dystrophin function is restored by exon 45 skipping.

The fusion protein may comprise a Cas protein and one or more base-editing domains. In some embodiments, the base-editing domain includes an adenine base editor (ABE). The fusion protein may comprise a Cas protein and one or more adenine base editor domains. Adenine base editors may include, for example, ecTadA, including wild-type and mutants thereof. Examples of ecTadA adenine base editors are included in the fusion proteins of SEQ ID NOs: 27-34 (annotated sequences of which are included herein). The adenine base editor may be as described in Gaudelli et al. (Nature 2017, 551, 464-471). Koblan et al. (Nature Biotech. 2018, 36, 843-846), Richter et al. (Nature Biotech. 2020, 38, 883-891), and Gaudelli et al. (Nature Biotech. 2020, 38, 892-900), each of which is incorporated herein by reference. The ABE may comprise a polypeptide selected from SEQ ID NOs: 45-52. The ABE may be encoded by a polynucleotide comprising a sequence selected from SEQ ID NOs: 53-80. In some embodiments, the ABE comprises an amino acid sequence of SEQ ID NO: 45, encoded by a polynucleotide sequence of SEQ ID NO: 53. In some embodiments, the ABE comprises an amino acid sequence of SEQ ID NO: 46, encoded by a polynucleotide sequence of SEQ ID NO: 54. In some embodiments, the ABE comprises an amino acid sequence of SEQ ID NO: 47, encoded by a polynucleotide sequence of SEQ ID NO: 55. In some embodiments, the ABE comprises an amino acid sequence of SEQ ID NO: 48, encoded by a polynucleotide sequence of SEQ ID NO: 56. In some embodiments, the ABE comprises an amino acid sequence of SEQ ID NO: 49, encoded by a polynucleotide sequence of SEQ ID NO: 57. In some embodiments, the ABE comprises an amino acid sequence of SEQ ID NO: 50, encoded by a polynucleotide sequence of SEQ ID NO: 58. In some embodiments, the ABE comprises an amino acid sequence of SEQ ID NO: 51, encoded by a polynucleotide sequence of SEQ ID NO: 59. In some embodiments, the ABE comprises an amino acid sequence of SEQ ID NO: 52, encoded by a polynucleotide sequence of SEQ ID NO: 60. In some embodiments, the fusion protein further can include at least one nuclear localization sequence (NLS), as detailed above. The at least one NLS may be at the N-terminal end of the fusion protein, at the C-terminal end of the protein, or a combination thereof.

In some embodiments, the fusion protein comprises a polypeptide selected from SEQ ID NOs: 27-34. In some embodiments, the fusion protein is encoded by a polynucleotide comprising a sequence selected from SEQ ID NOs: 35-42. In some embodiments, the fusion protein comprises the amino acid sequence of SEQ ID NO: 27, encoded by a polynucleotide sequence comprising SEQ ID NO: 35. In some embodiments, the fusion protein comprises the amino acid sequence of SEQ ID NO: 28, encoded by a polynucleotide sequence comprising SEQ ID NO: 36. In some embodiments, the fusion protein comprises the amino acid sequence of SEQ ID NO: 29, encoded by a polynucleotide sequence comprising SEQ ID NO: 37. In some embodiments, the fusion protein comprises the amino acid sequence of SEQ ID NO: 30, encoded by a polynucleotide sequence comprising SEQ ID NO: 38. In some embodiments, the fusion protein comprises the amino acid sequence of SEQ ID NO: 31, encoded by a polynucleotide sequence comprising SEQ ID NO: 39. In some embodiments, the fusion protein comprises the amino acid sequence of SEQ ID NO: 32, encoded by a polynucleotide sequence comprising SEQ ID NO: 40. In some embodiments, the fusion protein comprises the amino acid sequence of SEQ ID NO: 33, encoded by a polynucleotide sequence comprising SEQ ID NO: 41. In some embodiments, the fusion protein comprises the amino acid sequence of SEQ ID NO: 34, encoded by a polynucleotide sequence comprising SEQ ID NO: 42.

In some embodiments, the base-editing domain includes (i) a cytidine deaminase domain and (ii) at least one uracil glycosylase inhibitor (UGI) domain. The cytidine deaminase domain can convert the DNA base cytosine to uracil (see FIG. 1C). In some embodiments, the cytidine deaminase domain can include an apolipoprotein B mRNA-editing enzyme, catalytic polypeptide-like (APOBEC) family deaminase. In some embodiments, the cytidine deaminase domain can include an APOBEC 1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, APOBEC3H deaminase, or a combination thereof. In some embodiments, the cytidine deaminase domain comprises an APOBEC 1 deaminase. In some embodiments, the cytidine deaminase domain comprises a rat APOBEC 1 deaminase. In some embodiments, a cytidine deaminase enzyme (for example, rAPOBEC1) can be fused to the N-terminus of dCas to generate a base editing enzyme named BE1.

In some embodiments, the at least one UGI domain comprises a domain capable of inhibiting uracil-DNA glycosylases (UDG) activity. UDG activity may include eliminating uracil from nucleic acids by cleaving the N-glycosidic bond. UDG activity may initiate the base-excision repair (BER) pathway. The UGI domain that can inhibit UDG activity can prevent the subsequent U:G mismatch from being repaired back to a C:G base pair thus manipulating the cellular DNA repair processes and increasing the yield of the desired outcome (e.g., T:A base pair). In some embodiments, the at least one UGI domain comprises a polypeptide having an amino acid sequence of SEQ ID NO: 20. In some embodiments, the at least one UGI domain comprises an amino acid sequence encoded by the polynucleotide sequence of SEQ ID NO: 6 or SEQ ID NO: 18. In some embodiments, the base-editing domain comprises one UGI domain or two UGI domains. When more than one UGI domain is present in the base-editing domain, slightly different or variant sequences of the UGI domain may be used to avoid the tendency of two identical sequences to recombine when adjacent to each other on the same construct. In some embodiments, a UGI can be fused to a cytidine deaminase enzyme (e.g., rAPOBEC1) fused to the N-terminus of dCas to generate a base editing enzyme named BE2. In some embodiments, two UGI can be fused to a cytidine deaminase enzyme (e.g., rAPOBEC1) fused to the N-terminus of dCas to generate a base editing enzyme named BE4.

In some embodiments, the fusion protein can include the structure: NH₂-[cytidine deaminase domain]-[Cas protein]-[UGI domain]-COOH, and wherein each instance of “-” comprises an optional linker. In some embodiments, the fusion protein can include the structure: NH₂-[cytidine deaminase domain]-[Cas protein]-[UGI domain]-[UGI domain]-COOH, and wherein each instance of “-” comprises an optional linker. In some embodiments, the fusion protein can include the structure: NH₂-[ABE]-[Cas protein]-COOH, and wherein each instance of “-” comprises an optional linker. In some embodiments, the fusion protein can include the structure: NH₂-[Cas protein]-[ABE]-COOH, and wherein each instance of “-” comprises an optional linker. In some embodiments, the fusion protein can include the structure: NH₂-[ABE]-[ABE]-[Cas protein]-COOH, and wherein each instance of “-” comprises an optional linker. A linker may be any sequence of amino acids. A linker may be, for example, about 2-10, about 5-10, about 5-20, or about 10-25 amino acids in length. A linker may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids in length. A linker may be less than 30, less than 29, less than 28, less than 27, less than 26, less than 25, less than 24, less than 23, less than 22, less than 21, less than 20, less than 19, less than 18, less than 17, less than 16, less than 15, less than 14, less than 13, less than 12, less than 11, or less than 10 amino acids in length. In some embodiments, the linker comprises a XTEN linker (16 amino acids). In some embodiments, the linker comprises an amino acid sequence of SEQ ID NO: 61 or SEQ ID NO: 62, encoded by a polynucleotide sequence of SEQ ID NO: 63 or SEQ ID NO: 64, respectively. In some embodiments, the fusion protein further can include a nuclear localization sequence (NLS). In some embodiments, the fusion protein comprises the structure: NH₂-[cytidine deaminase domain]-[Cas9 protein]-[UGI domain]-[NLS]-COOH, and wherein each instance of “-” comprises an optional linker. In some embodiments, the fusion protein can include the structure: NH₂-[NLS]-[ABE]-[Cas protein]-COOH, and wherein each instance of “-” comprises an optional linker. In some embodiments, the fusion protein can include the structure: NH₂-[ABE]-[Cas protein]-[NLS]-COOH, and wherein each instance of “-” comprises an optional linker. In some embodiments, the fusion protein can include the structure: NH₂-[NLS]-[ABE]-[Cas protein]-[NLS]-COOH, and wherein each instance of “-” comprises an optional linker. In some embodiments, the fusion protein can include the amino acid sequence encoded by or corresponding to SEQ ID NO: 7 or SEQ ID NO: 8 or any of SEQ ID NOs: 27-34.

c. gRNA

The CRISPR/Cas-based base editing system may include at least one gRNA. The gRNA may target the dystrophin gene. The gRNA may bind and target a portion of the dystrophin gene. The gRNA may target an RNA splice site in the dystrophin gene. The gRNA may target an RNA splice site in a mutated dystrophin gene. The gRNA provides the targeting of the CRISPR/Cas-based base editing systems. The gRNA is a fusion of two noncoding RNAs: a crRNA and a tracrRNA. The gRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9.

The “target region” or “target sequence” or “protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds: “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 74 (RNA), which is encoded by a sequence comprising SEQ ID NO: 73 (DNA). The CRISPR/Cas9-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

In some embodiments, at least one gRNA may target and bind a target region. In some embodiments, between 1 and 20 gRNAs may be used to alter a target gene, for example, to alter a splice acceptor site. For example, between 1 gRNA and 20 gRNAs, between 1 gRNA and 15 gRNAs, between 1 gRNA and 10 gRNAs, between 1 gRNA and 5 gRNAs, between 2 gRNAs and 20 gRNAs, between 2 gRNAs and 15 gRNAs, between 2 gRNAs and 10 gRNAs, between 2 gRNAs and 5 gRNAs, between 5 gRNAs and 20 gRNAs, between 5 gRNAs and 15 gRNAs, or between 5 gRNAs and 10 gRNAs may be included in the CRISPR/Cas-based base editing system and used to alter the splice acceptor site. In some embodiments, at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNAs, at least 12 gRNAs, at least 13 gRNAs, at least 14 gRNAs, at least 15 gRNAs, or at least 20 gRNAs may be included in the CRISPR/Cas-based base editing system and used to alter the splice acceptor site. In some embodiments, less than 20 gRNAs, less than 19 gRNAs, less than 18 gRNAs, less than 17 gRNAs, less than 16 gRNAs, less than 15 gRNAs, less than 14 gRNAs, less than 13 gRNAs, less than 12 gRNAs, less than 11 gRNAs, less than 10 gRNAs, less than 9 gRNAs, less than 8 gRNAs, less than 7 gRNAs, less than 6 gRNAs, less than 5 gRNAs, less than 4 gRNAs, or less than 3 gRNAs may be included in the CRISPR/Cas-based base editing system and used to alter the splice acceptor site.

The CRISPR/Cas-based base editing system may use gRNA of varying sequences and lengths. The gRNA may comprise a complementary polynucleotide sequence of the target DNA sequence, such as a target sequence comprising SEQ ID NO: 1 or one of SEQ ID NOs: 21-23 or 43 or a complementary polynucleotide sequence of a target sequence comprising SEQ ID NO: 1 or one of SEQ ID NOs: 21-23 or 43, followed by NGG. The gRNA may comprise a “G” at the 5 end of the complementary polynucleotide sequence. The gRNA may comprise a 5-40 base pair, 5-35 base pair, 5-30 base pair, 10-35 base pair, or 10-30 base pair complementary polynucleotide sequence of the target DNA sequence followed by NGG. The gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by NGG. The gRNA may comprise a less than 40 base pair, less than 35 base pair, less than 30 base pair, less than 25 base pair, less than 24 base pair, less than 23 base pair, less than 22 base pair, less than 21 base pair, less than 20 base pair, less than 19 base pair, less than 18 base pair, at less than 17 base pair, less than 16 base pair, or less than 15 base pair complementary polynucleotide sequence of the target DNA sequence followed by NGG. The gRNA may target at least one of the promoter region, the enhancer region, or the transcribed region of the target gene.

The at least one gRNA may target a nucleic acid sequence comprising SEQ ID NO: 1. In some embodiments, the at least one gRNA is encoded by a nucleic acid sequence comprising SEQ ID NO: 1. The gRNA may target a sequence comprising at least one of SEQ ID NOs: 21-23 or 43 or a complement thereof, a variant thereof, or a fragment thereof. The gRNA may comprise a sequence selected from SEQ ID NOs: 24-26 or 44 or a complement thereof, a variant thereof, or a fragment thereof. The gRNA may include a nucleic acid sequence corresponding to at least one of SEQ ID NO: 1, a complement thereof, a variant thereof, or fragment thereof.

### 3. COMPOSITIONS FOR RESTORING DYSTROPHIN FUNCTION

The present invention is directed to a composition for restoring dystrophin function by altering or eliminating a splice acceptor site of exon 45. The composition may include the CRISPR/Cas-based base editing system, as disclosed above. The composition may also include a viral delivery system. For example, the viral delivery system may include an adeno-associated virus vector or a modified lentiviral vector.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery.

a. Constructs and Plasmids

The compositions, as described above, may comprise genetic constructs that encodes the CRISPR/Cas-based base editing system, as disclosed herein. The genetic construct, such as a plasmid or expression vector, may comprise a nucleic acid that encodes the CRISPR/Cas-based base editing system and/or at least one of the gRNAs. The compositions, as described above, may comprise genetic constructs that encodes the modified Adeno-associated virus (AAV) vector and a nucleic acid sequence that encodes the CRISPR/Cas-based base editing system, as disclosed herein. In some embodiments, the compositions, as described above, may comprise genetic constructs that encodes the modified adenovirus vector and a nucleic acid sequence that encodes the CRISPR/Cas-based base editing system, as disclosed herein. The genetic construct, such as a plasmid, may comprise a nucleic acid that encodes the CRISPR/Cas-based base editing system. The compositions, as described above, may comprise genetic constructs that encodes a modified lentiviral vector. The genetic construct, such as a plasmid, may comprise a nucleic acid that encodes the fusion protein and the at least one gRNA. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

The nucleic acid sequences may make up a genetic construct that may be a vector. The vector may be capable of expressing the fusion protein, such as the CRISPR/Cas-based base editing system, in the cell of a mammal. The vector may be recombinant. The vector may comprise heterologous nucleic acid encoding the fusion protein, such as the CRISPR/Cas-based base editing system. The vector may be a plasmid. The vector may be useful for transfecting cells with nucleic acid encoding the CRISPR/Cas-based base editing system, which the transformed host cell is cultured and maintained under conditions wherein expression of the CRISPR/Cas-based base editing system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cas-based base editing system and may further comprise an initiation codon, which may be upstream of the CRISPR/Cas-based base editing system coding sequence, and a stop codon, which may be downstream of the CRISPR/Cas-based base editing system coding sequence. The initiation and termination codon may be in frame with the CRISPR/Cas-based base editing system coding sequence. The vector may also comprise a promoter that is operably linked to the CRISPR/Cas-based base editing system coding sequence. The CRISPR/Cas-based base editing system may be under the light-inducible or chemically inducible control to enable the dynamic control of base editing in space and time. The promoter operably linked to the CRISPR/Cas-based base editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication No. US20040175727, the contents of which are incorporated herein in its entirety.

The vector may also comprise a polyadenylation signal, which may be downstream of the CRISPR/Cas-based base editing system. The polyadenylation signal may be a SV40 polyadenylation signal, LTR polyadenylation signal, bovine growth hormone (bGH) polyadenylation signal, human growth hormone (hGH) polyadenylation signal, or human-globin polyadenylation signal. The SV40 polyadenylation signal may be a polyadenylation signal from a pCEP4 vector (Invitrogen, San Diego, CA).

The vector may also comprise an enhancer upstream of the CRISPR/Cas-based base editing system or sgRNAs. The enhancer may be necessary for DNA expression. The enhancer may be human actin, human myosin, human hemoglobin, human muscle creatine or a viral enhancer such as one from CMV, HA, RSV or EBV. Polynucleotide function enhancers are described in U.S. Pat. Nos. 5,593,972, 5,962,428, and WO94/016737, the contents of each are fully incorporated by reference. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is incorporated fully by reference. In some embodiments the vector may comprise the nucleic acid sequence encoding the CRISPR/Cas-based base editing system, including the nucleic acid sequence encoding the fusion protein and the nucleic acid sequence encoding the at least one gRNA comprising the nucleic acid sequence of SEQ ID NO: 1, a complement thereof, a variant thereof, or a fragment thereof.

In some embodiments, the compositions are delivered by mRNA and protein/RNA complexes (Ribonucleoprotein (RNP)). For example, the purified fusion protein can be combined with guide RNA to form an RNP complex.

b. Modified Lentiviral Vector

The compositions for altering splice acceptor sites of exon 45 may include a modified lentiviral vector. The modified lentiviral vector includes a first polynucleotide sequence encoding a fusion protein and a second polynucleotide sequence encoding the at least one gRNA. The first polynucleotide sequence may be operably linked to a promoter. The promoter may be a constitutive promoter, an inducible promoter, a repressible promoter, or a regulatable promoter.

The second polynucleotide sequence encodes at least 1 gRNA. For example, the second polynucleotide sequence may encode between 1 gRNA and 20 gRNAs, between 1 gRNA and 15 gRNAs, between 1 gRNA and 10 gRNAs, between 1 gRNA and 5 gRNAs, between 2 gRNAs and 20 gRNAs, between 2 gRNAs and 15 gRNAs, between 2 gRNAs and 10 gRNAs, between 2 gRNAs and 5 gRNAs, between 5 gRNAs and 20 gRNAs, between 5 gRNAs and 15 gRNAs, or between 5 gRNAs and 10 gRNAs. The second polynucleotide sequence may encode at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNA, at least 12 gRNAs, at least 13 gRNAs, at least 14 gRNAs, at least 15 gRNAs, at least 16 gRNAs, at least 17 gRNAs, at least 18 gRNAs, at least 19 gRNAs, or at least 20 gRNAs. The second polynucleotide sequence may encode less than 20 gRNAs, less than 19 gRNAs, less than 18 gRNAs, less than 17 gRNAs, less than 16 gRNAs, less than 15 gRNAs, less than 14 gRNAs, less than 13 gRNAs, less than 12 gRNAs, less than 11 gRNAs, less than 10 gRNAs, less than 9 gRNAs, less than 8 gRNAs, less than 7 gRNAs, less than 6 gRNAs, less than 5 gRNAs, less than 4 gRNAs, or less than 3 gRNAs. The second polynucleotide sequence may be operably linked to a promoter. The promoter may be a constitutive promoter, an inducible promoter, a repressible promoter, or a regulatable promoter. At least one gRNA may bind to a target gene or loci, such as a target region comprising the exon 45 splice acceptor site.

c. Adeno-Associated Virus Vectors

AAV may be used to deliver the compositions to the cell using various construct configurations. For example, AAV may deliver the fusion protein and the gRNA expression cassettes on separate vectors. Alternatively, both the fusion protein and up to two gRNA expression cassettes may be combined in a single AAV vector within the 4.7 kb packaging limit.

The composition, as described above, includes a modified adeno-associated virus (AAV) vector. The modified AAV vector may be capable of delivering and expressing the site-specific nuclease in the cell of a mammal. For example, the modified AAV vector may be an AAV-SASTG vector (Piacentino et al. (2012) Human Gene Therapy 23:635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5 and AAV/SASTG vectors that efficiently transduce skeletal muscle or cardiac muscle by systemic and local delivery (Seto et al. Current Gene Therapy 2012, 12, 139-151).

### 4. METHODS OF RESTORING DYSTROPHIN FUNCTION IN A SUBJECT HAVING A MUTANT DYSTROPHIN GENE

Provided herein are methods of restoring dystrophin function (e.g., a mutant dystrophin gene, e.g., a mutant human dystrophin gene) in a cell and/or a subject suffering from DMD and/or having a mutant dystrophin gene. Also provided herein are methods of treating Duchenne Muscular Dystrophy in a subject in need thereof. Also provided herein are methods of altering an RNA splice site encoded in the genomic DNA of a subject. The method can include administering to a cell or subject or cell thereof a CRISPR/Cas-based gene editing system, a polynucleotide or vector encoding said CRISPR/Cas-based gene editing system, or composition of said CRISPR/Cas9-based gene editing system as detailed herein. In some embodiments, the subject is suffering from Duchenne Muscular Dystrophy

The method can include administering to a cell or a subject a presently disclosed genetic construct (e.g., a vector) or a composition comprising thereof as described above. The method can comprises administering to the skeletal muscle or cardiac muscle of the subject the presently disclosed genetic construct (e.g., a vector) or a composition comprising thereof for genome editing, for example base editing, in skeletal muscle or cardiac muscle, as described above. Use of presently disclosed genetic construct (e.g., a vector) or a composition comprising thereof to deliver the CRISPR/Cas-based gene editing system to the skeletal muscle or cardiac muscle may restore the expression of a full-functional or partially-functional protein. The CRISPR/Cas-based gene editing system has the advantage of advanced genome editing due to their high rate of successful and efficient genetic modification.

The method may include administering a CRISPR/Cas-based gene editing system, such as administering a fusion protein, a polynucleotide sequence encoding said fusion protein and/or at least one gRNA comprising or encoded by or corresponding to SEQ ID NO: 1, a complement thereof, a variant thereof, or fragment thereof.

### 5. PHARMACEUTICAL COMPOSITIONS

The CRISPR/Cas-based base editing system may be in a pharmaceutical composition. The pharmaceutical composition may comprise about 1 ng to about 10 mg of DNA encoding the CRISPR/Cas-based base editing system. The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The pharmaceutical composition containing the CRISPR/Cas-based base editing system may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the pharmaceutical composition containing the CRISPR/Cas-based base editing system at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the CRISPR/Cas-based base editing system may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

### 6. METHODS OF DELIVERY

Provided herein is a method for delivering the pharmaceutical formulations of the CRISPR/Cas-based base editing system for providing genetic constructs and/or proteins of the CRISPR/Cas-based base editing system. The delivery of the CRISPR/Cas-based base editing system may be the transfection or electroporation of the CRISPR/Cas-based base editing system as one or more nucleic acid molecules that is expressed in the cell and delivered to the surface of the cell. The CRISPR/Cas-based base editing system protein may be delivered to the cell. The nucleic acid molecules may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The vector encoding a CRISPR/Cas-based base editing system protein may be delivered to the mammal by DNA injection (also referred to as DNA vaccination) with and without in vivo electroporation, liposome mediated, nanoparticle facilitated, and/or recombinant vectors. The recombinant vector may be delivered by any viral mode. The viral mode may be recombinant lentivirus, recombinant adenovirus, and/or recombinant adeno-associated virus.

The polynucleotide encoding a CRISPR/Cas-based base editing system protein may be introduced into a cell to induce gene expression of the target gene. For example, one or more polynucleotide sequences encoding the CRISPR/Cas-based base editing system directed towards a target gene may be introduced into a mammalian cell. Upon delivery of the CRISPR/Cas-based base editing system to the cell, and thereupon the vector into the cells of the mammal, the transfected cells will express the CRISPR/Cas-based base editing system. The CRISPR/Cas-based base editing system may be administered to a mammal to induce or modulate gene expression of the target gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

Upon delivery of the presently disclosed genetic construct or composition to the tissue, and thereupon the vector into the cells of the mammal, the transfected cells will express the gRNA molecule(s) and the Cas9 molecule. The genetic construct or composition may be administered to a mammal to alter gene expression or to re-engineer or alter the genome. For example, the genetic construct or composition may be administered to a mammal to restore dystrophin function in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

The genetic construct (for example, a vector) encoding the gRNA molecule(s) and the Cas9 molecule can be delivered to the mammal by DNA injection (also referred to as DNA vaccination) with and without in vivo electroporation, liposome mediated, nanoparticle facilitated, and/or recombinant vectors. The recombinant vector can be delivered by any viral mode. The viral mode can be recombinant lentivirus, recombinant adenovirus, and/or recombinant adeno-associated virus.

A presently disclosed genetic construct (for example, a vector) or a composition comprising thereof can be introduced into a cell to genetically restore dystrophin function of a dystrophin gene (for example, human dystrophin gene). In certain embodiments, a presently disclosed genetic construct (for example, a vector) or a composition comprising thereof is introduced into a myoblast cell from a DMD patient. In certain embodiments, the genetic construct (for example, a vector) or a composition comprising thereof is introduced into a fibroblast cell from a DMD patient, and the genetically corrected fibroblast cell can be treated with MyoD to induce differentiation into myoblasts, which can be implanted into subjects, such as the damaged muscles of a subject to verify that the corrected dystrophin protein is functional and/or to treat the subject. The modified cells can also be stem cells, such as induced pluripotent stem cells, bone marrow-derived progenitors, skeletal muscle progenitors, human skeletal myoblasts from DMD patients, CD 133⁺ cells, mesoangioblasts, and MyoD- or Pax7-transduced cells, or other myogenic progenitor cells. For example, the CRISPR/Cas-based gene editing system may cause neuronal or myogenic differentiation of an induced pluripotent stem cell.

### 7. ROUTES OF ADMINISTRATION

The CRISPR/Cas-based base editing system and compositions thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The CRISPR/Cas-based base editing system and compositions thereof may be administered by traditional syringes, needleless injection devices, “microprojectile bombardment gone guns,” or other physical methods such as electroporation (“EP”), “hydrodynamic method”, or ultrasound. The composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus.

The presently disclosed genetic constructs (for example, vectors) or a composition comprising thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. In certain embodiments, the presently disclosed genetic construct (for example, a vector) or a composition is administered to a subject (for example, a subject suffering from DMD) intramuscularly, intravenously or a combination thereof. For veterinary use, the presently disclosed genetic constructs (for example, vectors) or compositions may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gone guns”, or other physical methods such as electroporation (“EP”), “hydrodynamic method”, or ultrasound.

The presently disclosed genetic construct (for example, a vector) or a composition may be delivered to the mammal by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior muscle or tail.

In some embodiments, the presently disclosed genetic construct (for example, a vector) or a composition thereof is administered by 1) tail vein injections (systemic) into adult mice; 2) intramuscular injections, for example, local injection into a muscle such as the TA or gastrocnemius in adult mice; 3) intraperitoneal injections into P2 mice; or 4) facial vein injection (systemic) into P2 mice.

### 8. CELL TYPES

Any of these delivery methods and/or routes of administration can be utilized for delivery of the herein described base editing system to a myriad of cell types. For example, cell types may include, but are not limited to, immortalized myoblast cells, such as wild-type and DMD patient derived lines, primary DMD dermal fibroblasts, induced pluripotent stem cells, bone marrow-derived progenitors, skeletal muscle progenitors, human skeletal myoblasts from DMD patients, CD 133⁺ cells, mesoangioblasts, cardiomyocytes, hepatocytes, chondrocytes, mesenchymal progenitor cells, hematopoetic stem cells, smooth muscle cells, and MyoD- or Pax7-transduced cells, or other myogenic progenitor cells. Immortalization of human myogenic cells can be used for clonal derivation of genetically corrected myogenic cells. Cells can be modified ex vivo to isolate and expand clonal populations of immortalized DMD myoblasts that include a genetically corrected or restored dystrophin gene and are free of other nuclease-introduced mutations in protein coding regions of the genome. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

### 9. KITS

Provided herein is a kit, which may be used to correct a mutated dystrophin gene and/or restore dystrophin function. The kit comprises at least one gRNA that binds and targets or is encoded by or is corresponding to a polynucleotide sequence of SEQ ID NO: 1, a complement thereof, a variant thereof, or fragment thereof, for restoring dystrophin function and instructions for using the CRISPR/Cas-based editing system. Also provided herein is a kit, which may be used for base editing of a dystrophin gene in skeletal muscle or cardiac muscle. The kit comprises genetic constructs (for example, vectors) or a composition comprising thereof for genome editing, for example base editing, in skeletal muscle or cardiac muscle, as described above, and instructions for using said composition.

Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (for example, magnetic discs, tapes, cartridges, chips), optical media (for example, CD ROM), and the like. As used herein, the term “instructions” may include the address of an internet site that provides the instructions.

The genetic constructs (for example, vectors) or a composition comprising thereof for restoring dystrophin function in skeletal muscle or cardiac muscle may include a modified AAV vector that includes a gRNA molecule(s) and the fusion protein, as described above, that specifically binds and cleaves a region of the dystrophin gene. The CRISPR/Cas-based gene editing system, as described above, may be included in the kit to specifically bind and target a particular region, for example the exon 45 splice acceptor containing region, in the mutated dystrophin gene.

### 10. EXAMPLES

The foregoing may be better understood by reference to the following examples, which are presented for purposes of illustration and are not intended to limit the scope of the invention. The present invention has multiple aspects, illustrated by the following non-limiting examples.

### Example 1

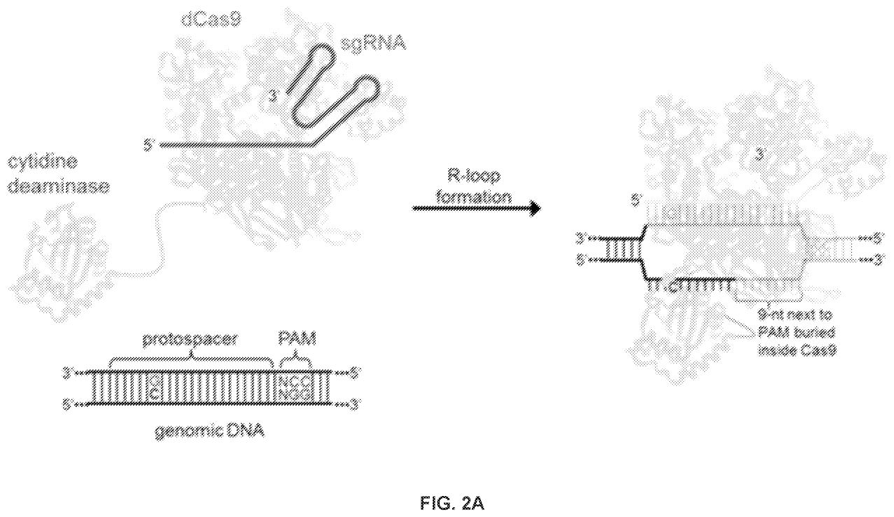

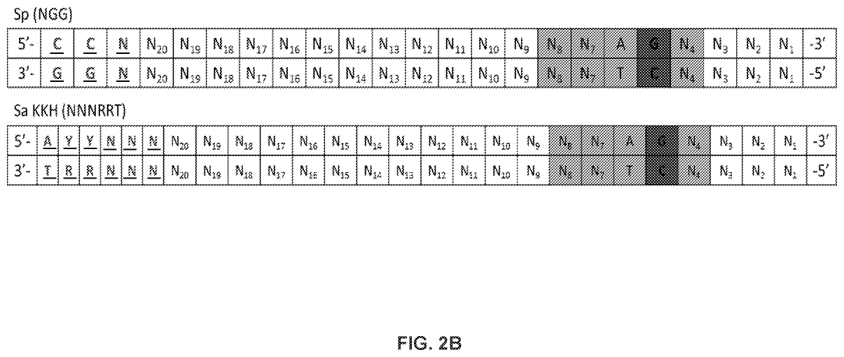

gRNAs were designed to base edit splice acceptors based on the availability of a PAM (see FIG. 2A and FIG. 2B). gRNAs were designed to target the DNA base editor systems with both S. pyogenes and S. aureus Cas9 proteins (FIG. 1A and FIG. 1B) to human dystrophin exons within the hotspot for deletions in the DMD gene between exons 45 and 55. The BE4max (Addgene #112093) and AncBE4max (Addgene #112094) designs, as described in FIG. 1B, worked better at lower plasmid concentrations than the designs in FIG. 1A, which had limited expression levels. The BE4max and AncBE4max designs performed similarly. As the gRNAs are binding to the Cas9 portion, which is constant between all designs, the same gRNA can be used through multiple generations of base editor (as long as the Cas9 species remains the same).

Splice acceptor G>A base editing were assayed at various dystrophin exons by plasmid transfection (Lipofectamine 2000) of human HEK293T cells with 400 ng of gRNA plasmid and 400 ng of BE4max or AncBE4max plasmid. Deep sequencing of the target sites using the MiSeq system (Illumina) was performed to determine the % G>A base editing. See TABLE 1. While some exons showed poor editing efficiency (i.e., <0.1% editing), 7-8% of alleles were observed to be edited at exon 45 using an exon 45 gRNA sequence of 5′-GTTCCTGTAAGATACCAAAA-3′ (SEQ ID NO: 1). Exon 45 is the dystrophin exon whose removal could treat the second largest group of DMD patients (˜8%) (Aartsma-Rus et al. Human Mutation 2009, 30, 293-299).

TABLE 1

| | Splice | % mutations | % G >A |
|---|---|---|---|
| Base Editor | Acceptor | treated by skipping | Editing |
| (PAM) | Target | this exon (ranking) | (HEK293T) |
| SpBE3 | Exon 44 | 6.2% (4^{th}) | 0.221% |
| (NGG) | Exon 45 | 8.1% (2^{nd}) | 2.174% |
| SaKKH-BE3 | Exon 44 | 6.2% (4^{th}) | 0.004% |
| (NNNRRT) | Exon 53 | 7.7% (3^{rd}) | 0.081% |
| | Exon 46 | 4.3% (5^{th}) | 0.197% |
| | Mouse Exon 23 | — | 0.017% |

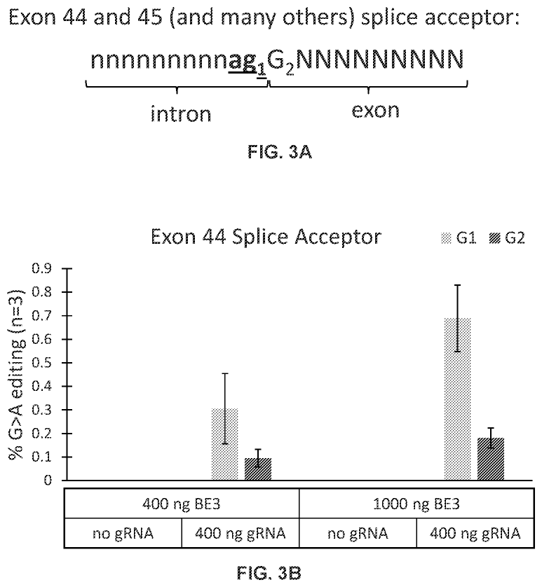

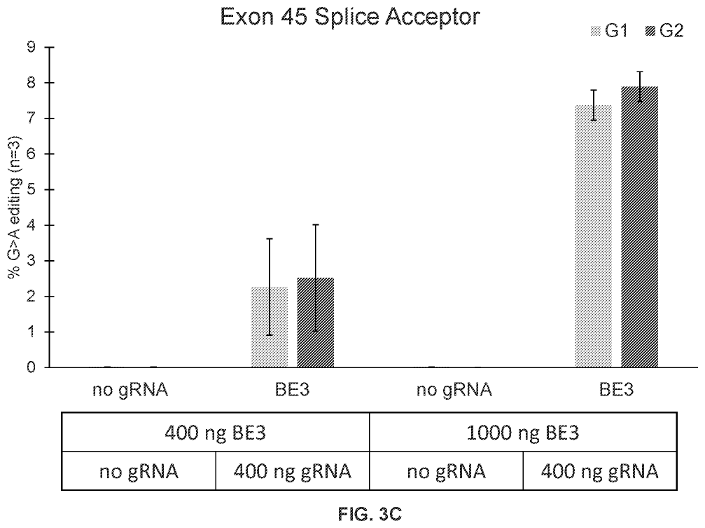

Splice acceptor G>A base editing were assayed at exons 44 and 45 by plasmid transfection (Lipofectamine 2000) of human HEK293T cells with 400 ng of gRNA plasmid and 400 ng or 1000 ng of the BE4max plasmid. Deep sequencing of the target sites using the MiSeq system (Illumina) was performed to determine the % G>A base editing. The transfection conditions were optimized by increasing the amount of BE3max plasmid to increase the base editing. As shown in FIG. 3B and FIG. 3C, the base editing was increased to 7-8% with exon 45 gRNA. Editing both the G1 and G2 as shown in FIG. 3A may provide proper exon skipping.

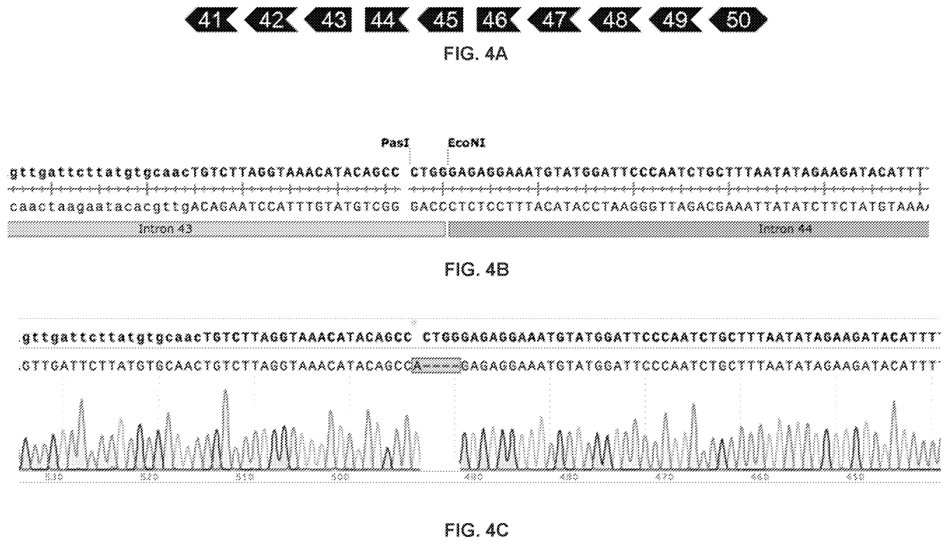

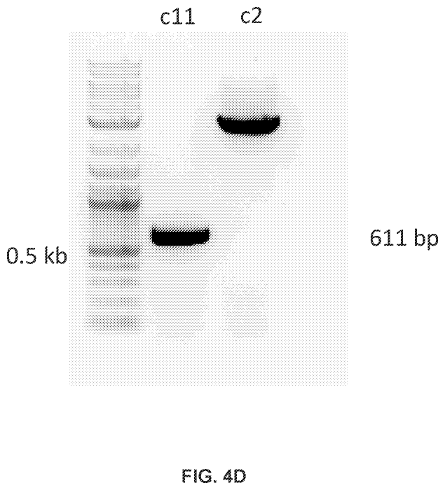

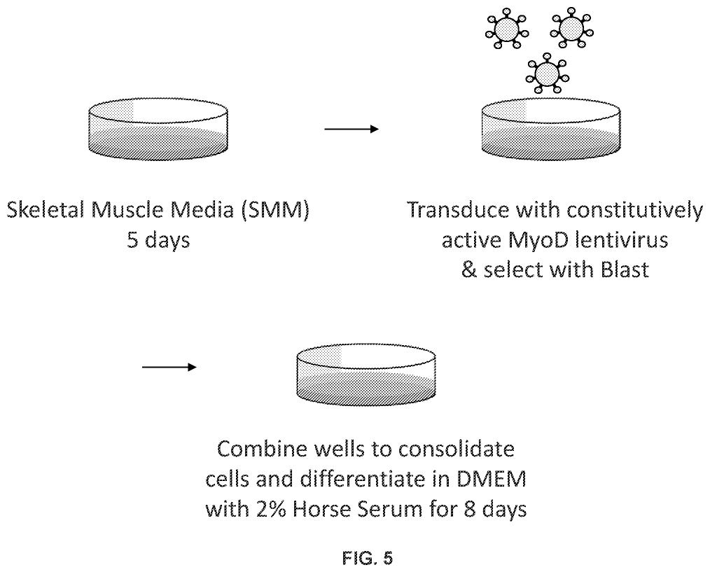

In order to test the effect of splice site disruption on exon skipping, a human induced pluripotent stem cell (iPSC) line harboring a deletion of dystrophin exon 44 was generated. See FIGS. 4A-4D. This pluripotent cell line models an inherited DMD mutation with a disrupted reading frame of the DMD gene that is correctable by removal of exon 45. iPSCs do not express dystrophin, so it is difficult to determine if the edited exon is getting skipped. Overexpression of MyoD in the iPSCs was used to express dystrophin to analyze the RNA and protein levels (FIG. 5).

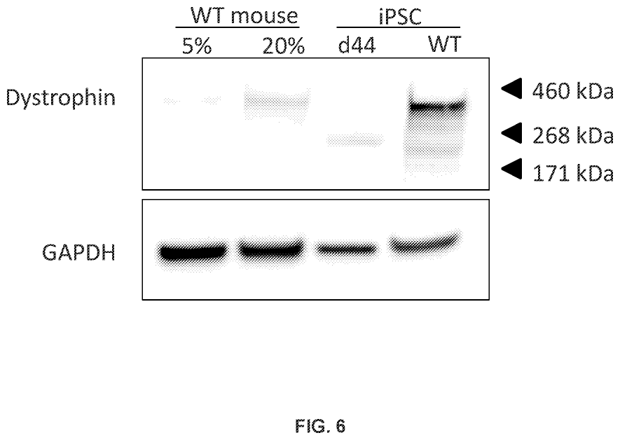

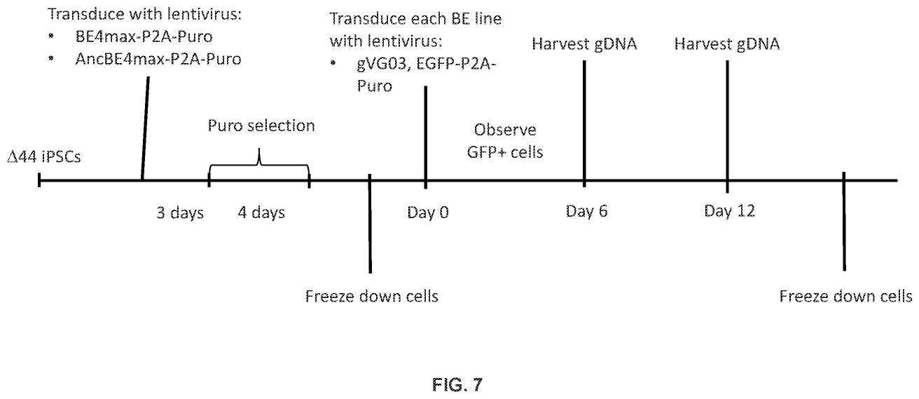

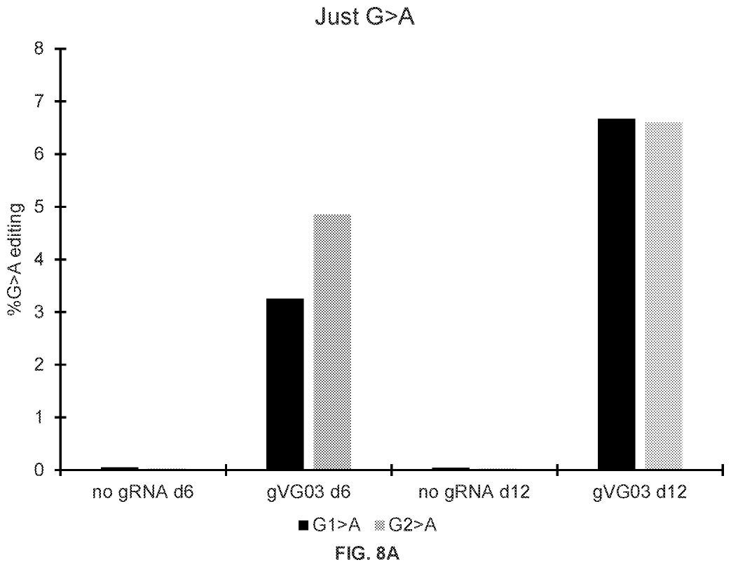

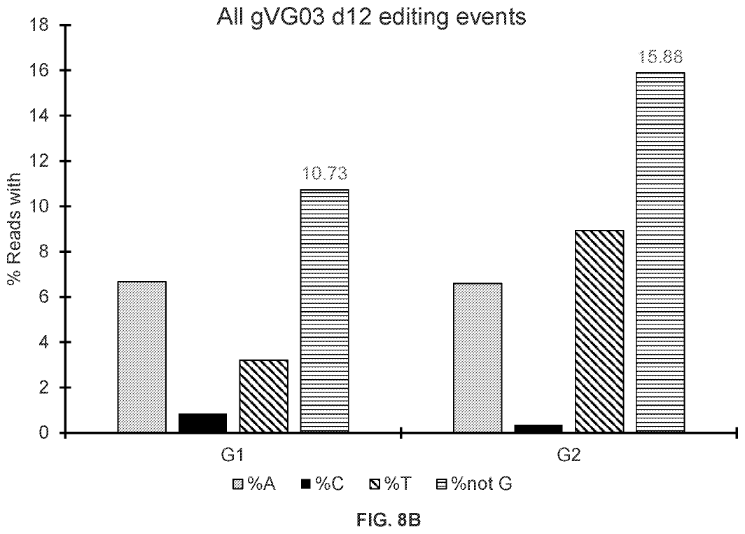

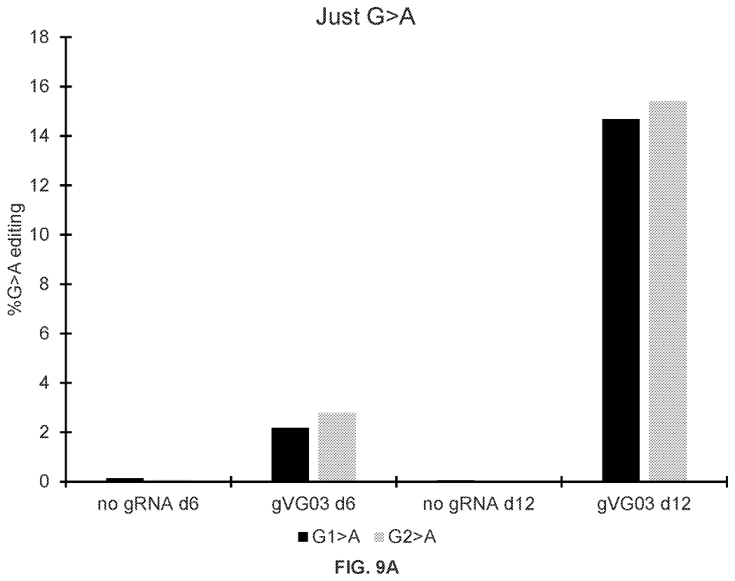

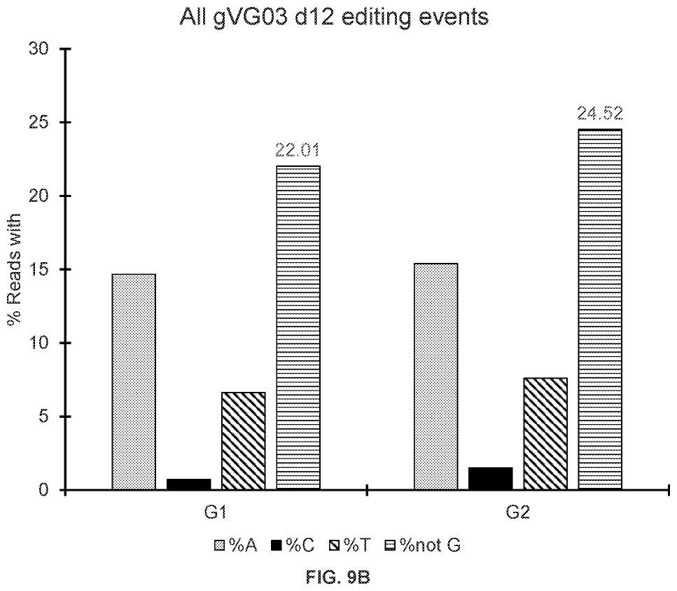

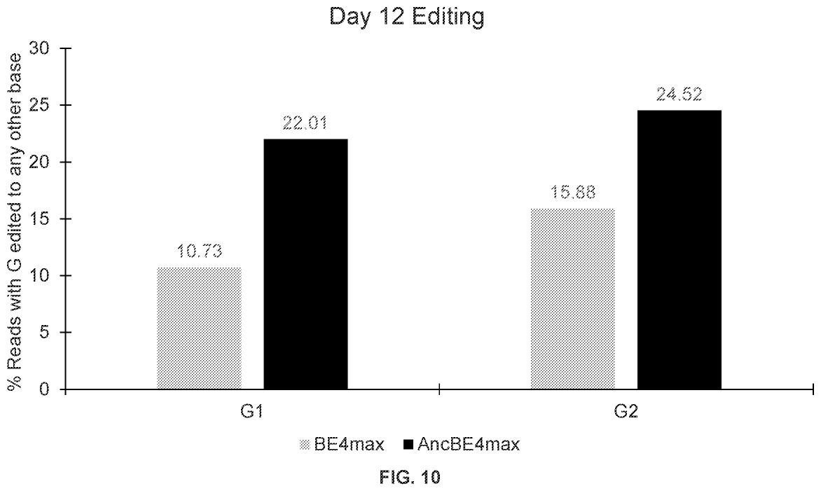

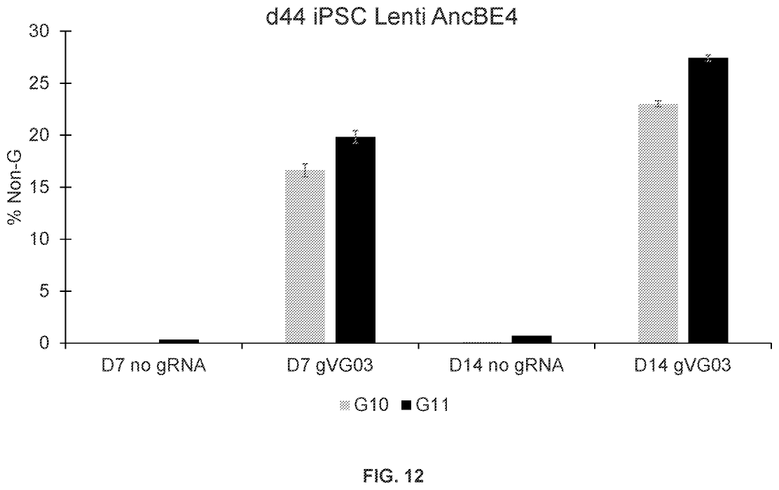

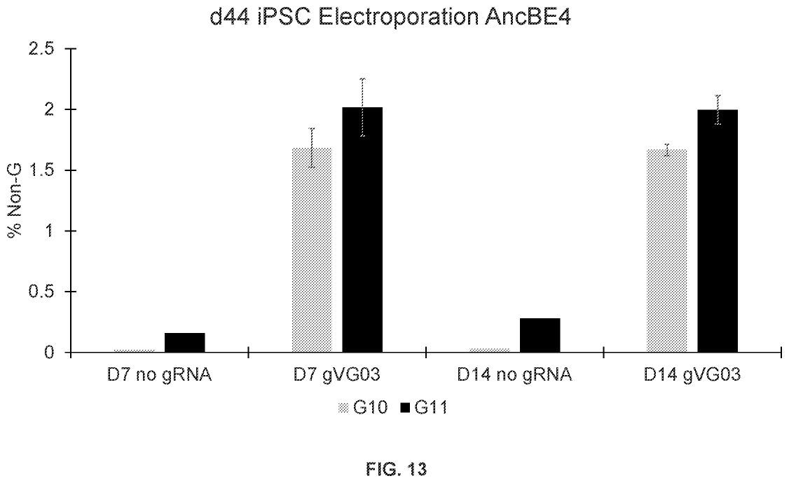

Myogenic differentiation of this Δ44 iPSC line by lentiviral transduction of MyoD cDNA confirms that the mutation ablates dystrophin protein expression. See FIG. 6. The S. pyogenes dCas9-based AncBE4max and a gRNA cassette was delivered to these cells by lentiviral transduction. FIG. 7 shows an outline of the procedure. 200 μL of 20× virus was used for BE4max and AncBE4 max transductions. FIG. 8A and FIG. 9A show the % G>A base editing events for BE4max and AncBE4max, respectively. FIG. 8B and FIG. 9B show all gVG03 d12 editing events for BE4max and AncBE4max, respectively. While the APOBEC enzyme in the construct design should convert G>A, sometimes G>T or G>C events also occur. Any of these cases that lead to the removal of the G should disrupt splicing, therefore the sum of “not G” events gives an effective editing rate. FIG. 10 shows Δ44 iPSC editing (% reads with G edited to any other base) after 12 days using BE4max and AncBE4max. Deep sequencing showed that 22% of splice acceptors were disrupted after 12 days. FIG. 12 shows % Non-G base editing events in the Δ44 iPSC using AncBE4max delivered by lentivrus. FIG. 13 shows % Non-G base editing events in the Δ44 iPSC using AncBE4max delivered by electroporation. The cells were harvested after being treated with the gRNA lentivirus for 7 days (D7) and 14 days (D14).

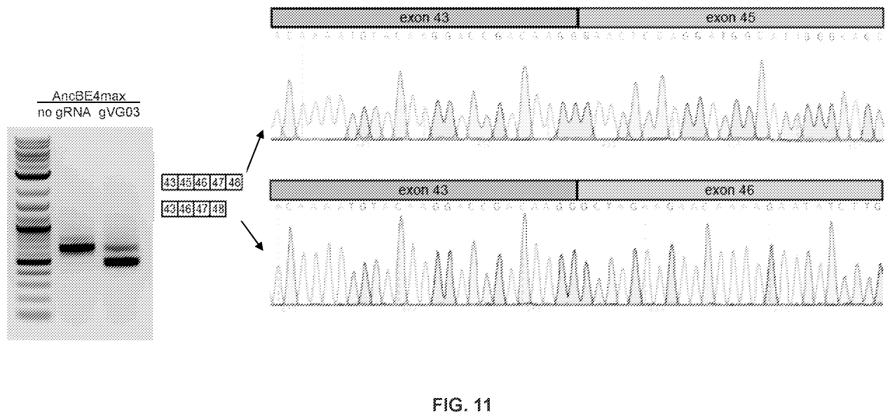

MyoD overexpression in this edited Δ44 iPSC line followed by RT-PCR confirmed that splice acceptor base editing results in skipping of exon 45, which restores the dystrophin reading frame. AncBE4max showed higher editing, so these edited cells were differentiated with MyoD and the RNA was harvested to look for skipping. FIG. 11 shows the RT-PCR results following 35 amplification cycles with the primers: 5′-CTACAACAAAGCTCAGGTCG-3′ (SEQ ID NO: 16) and 5′-TTCTCAGGTAAAGCTCTGGAAAC-3′ (SEQ ID NO: 17). Robust skipping of exon 45 was observed in cells that were treated with the exon 45 gRNA, but not in the no gRNA control.

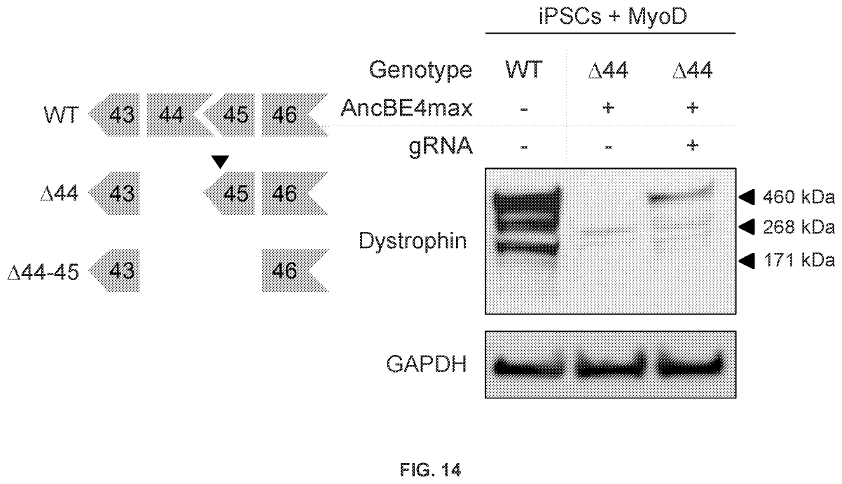

MyoD overexpression in this edited Δ44 iPSC line followed by Western blot analysis further confirmed that splice acceptor base editing results in skipping of exon 45, which restores the dystrophin reading frame. Δ44 iPSC cells transduced with AncBE4max lentivirus and gRNA lentivirus, or WT iPSCs, were differentiated with MyoD as above for FIG. 11. Cell lysates were harvested, and Western blot was performed with antibodies against dystrophin protein and GAPDH. The Western blot (FIG. 14) shows that while the untreated Δ44 iPSC cells had much reduced dystrophin protein expression, especially the largest isoform, base editing (with gRNA) was able to restore some dystrophin protein expression.

### Example 2

The removal of introns and inclusion of selected exons during mRNA splicing is critical to normal gene function and is often misregulated in genetic disorders. Technologies that modulate mRNA processing and exon selection, such as exon skipping approaches, may be used to study and treat these diseases. Exon skipping aims to restore the correct reading frame or induce alternative splicing by blocking the recognition of splicing sequences by the spliceosome, leading to removal of specific exons along with the adjacent introns. For example, Duchenne muscular dystrophy (DMD) is typically caused by deletions of one or more exons from the dystrophin gene, leading to disruption of the reading frame. Expression of dystrophin protein can be restored by correcting the reading frame by inducing the exclusion of one or more additional exons. By targeting Cas9 to the splice acceptor of exons, the indels produced during DNA repair can disrupt the splice site and induce exclusion of the exon. In contrast to the semi-random indels generated by the conventional CRISPR-Cas9 system, base editing technologies have been developed for the precise modification of a single base pair without inducing double-stranded DNA breaks. Adenine base editors can change an A directly to a G, or a T to C on the reverse strand, and they have been targeted to splice acceptor “AG” of a variety of exons to modulate mRNA splicing.

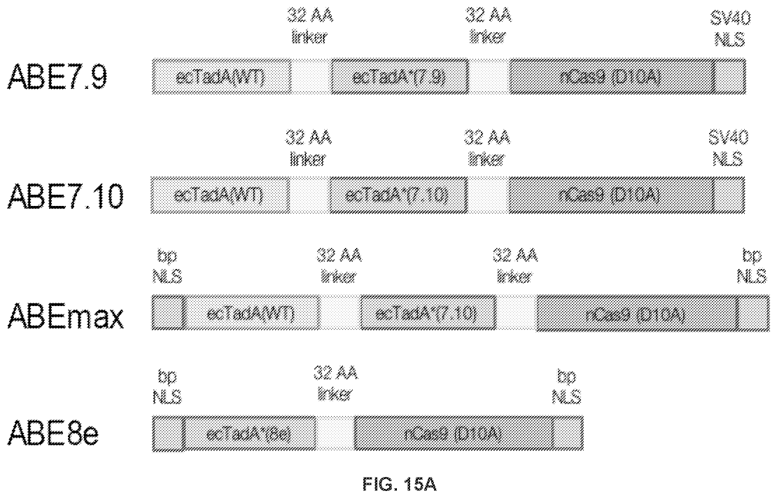

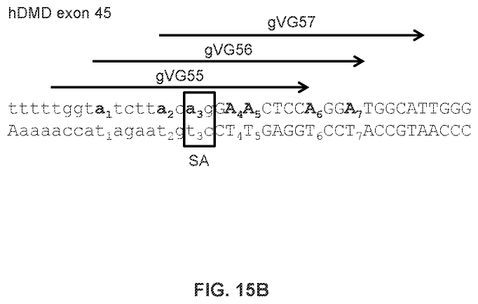

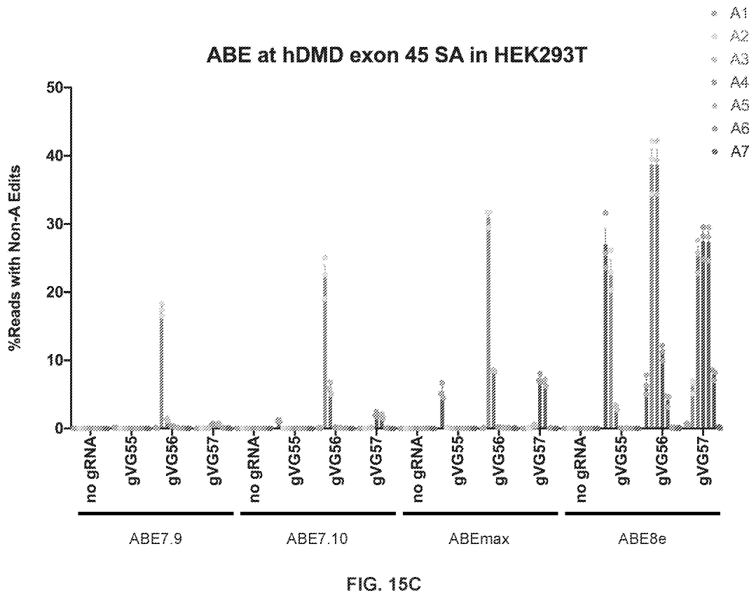

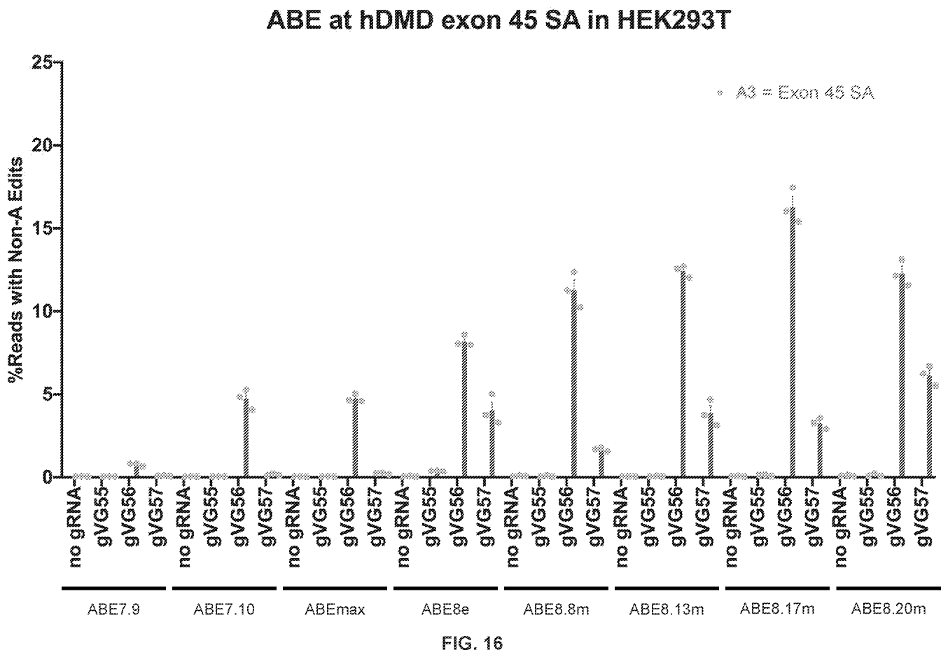

Guide RNAs were designed (gRNAs: TABLE 2) for 4 versions of adenine base editors (ABEs) constructed on S. pyogenes Cas9 targeting the splice acceptor (SA) of human dystrophin exon 45. Skipping exon 45 is applicable to treating the second largest group of DMD patients (8%), and the effect of base editing on dystrophin restoration can be tested in cell lines and mouse models. The four ABEs used were two different variants of the TadA enzyme (ABE7.9 and ABE7.10; Gaudelli et al. Nature 2017, 551, 464-471), a codon and NLS-optimized variant of ABE7.10 (ABEmax; Koblan et al. Nature Biotech. 2018, 36, 843-846), and a next generation evolution of ABEmax (ABE8e; Richter et al. Nature Biotech. 2020, 38, 883-891)(FIG. 15A). There are many adenines (A) that fall within the editing window of these three gRNAs, but the splice acceptor target that was edited for exon skipping was A3 (FIG. 15B). A transfection experiment was performed in HEK293T cells with 750 ng of ABE plasmid and 250 ng of gRNA plasmid. 30,000 HEK293 cells were plated in a 48-well. The next day, 750 ng base editor plasmid and 250 ng gRNA plasmid or pmaxGFP were transfected with Lipefectamine 2000. Quick extract was harvested 3 days after transfection, and editing was determined by deep sequencing and crispresso2. Results showed that after three days, ABE8e with gVG56 enabled conversion of 38.6% of the splice acceptor A3s to a non-A base, with G being the predominant edit (FIG. 15C). Next, this experiment was repeated with an expanded panel of four additional ABE variants, again with the same three gRNAs tested with each editor (Gaudelli et al. Nature Biotech. 2020, 38, 892-900)(FIG. 16). 30,000 HEK293 cells were plated in a 48-well. The next day, 750 ng base editor plasmid and 250 ng gRNA plasmid or pmaxGFP were transfected with Lipefectamine 2000. Quick extract was harvested 3 days after transfection, and editing was determined by deep sequencing and crispresso2. Across all variants tested, the gRNA gVG56 showed the greatest ability to edit the exon 45 splice acceptor (A3) compared to gVG55 and gVG56. The ABEs used in these experiments are included in the fusion proteins of SEQ ID NOs: 27-34. This editing strategy will be applied to an iPS cell line with an exon 44 deletion as well as a mouse containing the human dystrophin gene with an exon 44 deletion to show that base editing of the exon 45 splice acceptor will skip the exon and restore dystrophin expression.

TABLE 2

| gRNA | | |
|---|---|---|
| name | gRNA Sequence | gRNA |
| gVG55 | 5′-tggtatcttaca | 5′-ugguaucuuaca |
| (g01) | gGAACTCC-3′ | gGAACUCC-3′ |
| | (SEQ ID NO: 21) | (SEQ ID NO: 24) |
| gVG56 | 5′-atcttacagGAA | 5′-aucuuacagGAA |
| (g02) | CTCCAGGA-3′ | CUCCAGGA-3′ |
| | (SEQ ID NO: 22) | (SEQ ID NO: 25) |
| gVG57 | 5′-cagGAACTCCAG | 5′-cagGAACUCCAG |
| (g03) | GATGGCAT-3′ | GAUGGCAU-3′ |
| | (SEQ ID NO: 23) | (SEQ ID NO: 26) |
| g04 | 5′-GTTCctgtaaga | 5′-GUUCcuguaaga |
| | taccaaa-3′ | uaccaaa-3′ |
| | (SEQ ID NO: 43) | (SEQ ID NO: 44) |

### Example 3

### ABE8s Enable Efficient Exon 45 Splice Acceptor Editing in HEK293 Ts

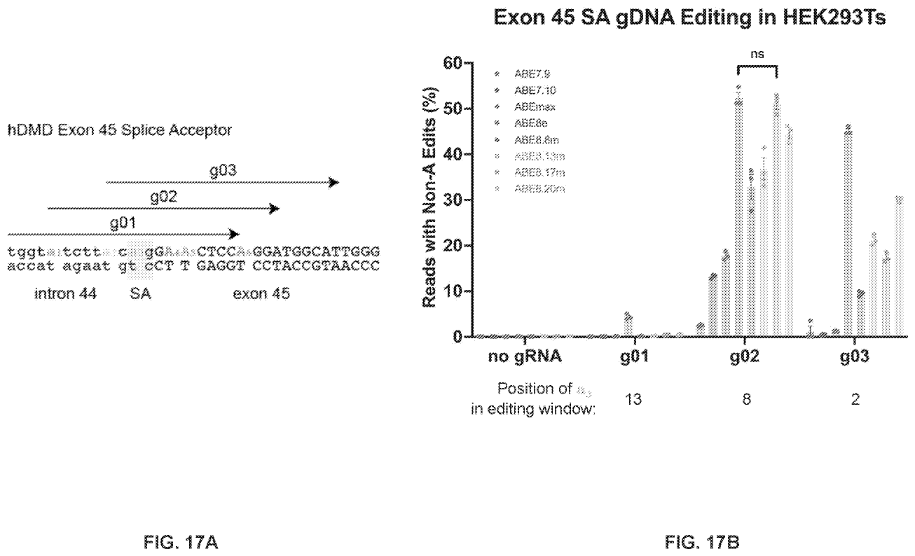

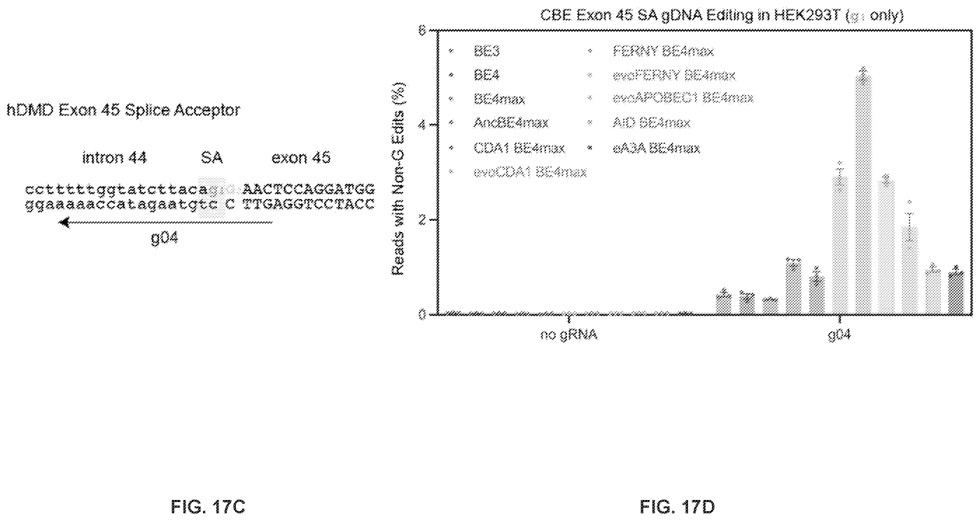

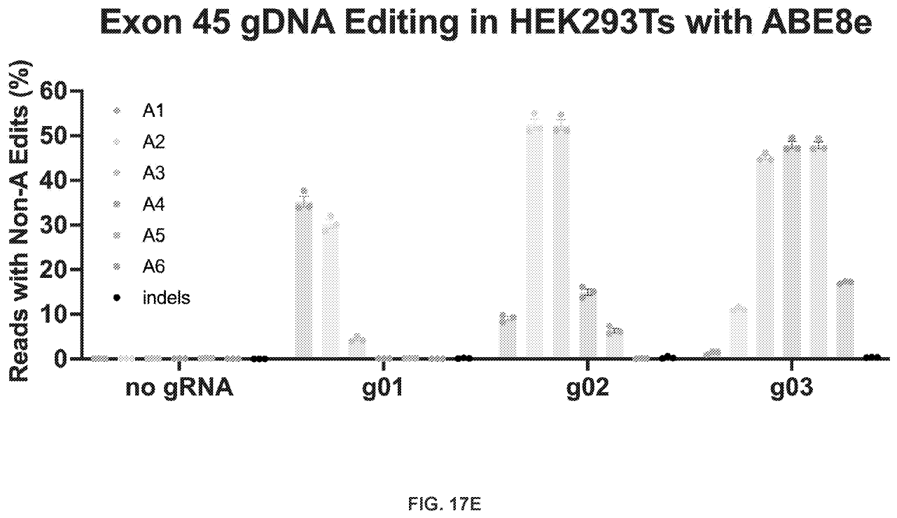

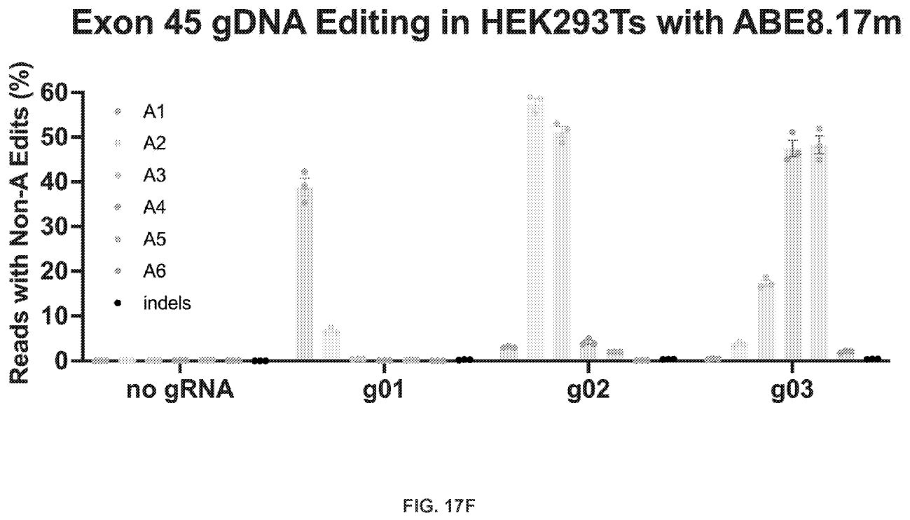

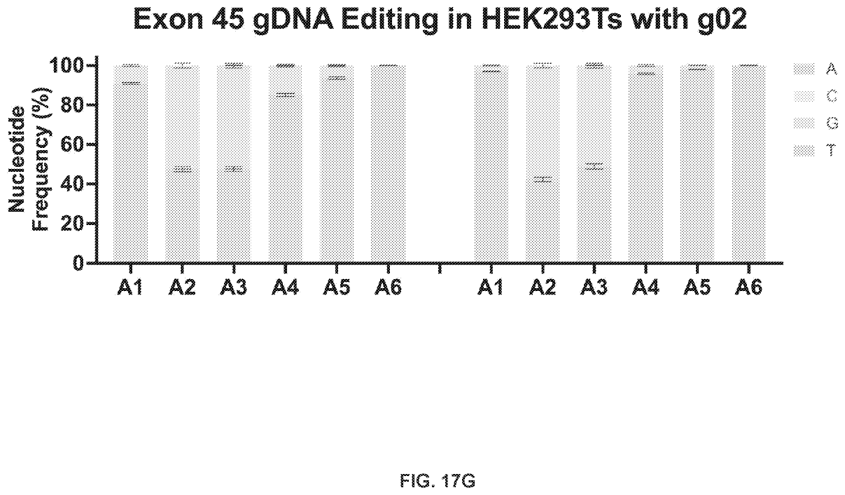

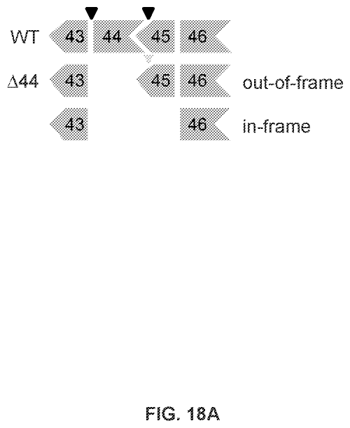

The gRNAs of Example 2 (gRNAs: TABLE 2, renamed g01, g02, and g03) and g04 were studied with additional versions of adenine base editors (ABEs) constructed on S. pyogenes Cas9 targeting the splice acceptor (SA) of human dystrophin exon 45. The ABEs used were two different variants of the TadA enzyme (ABE7.9 and ABE7.10; Gaudelli et al. Nature 2017, 551, 464-471), a codon and NLS-optimized variant of ABE7.10 (ABEmax; Koblan et al. Nature Biotech. 2018, 36, 843-848), a next generation evolution of ABEmax (ABE8e; Richter et al. Nature Biotech. 2020, 38, 883-891), ABE8.8m, ABE8.13m, ABE8.17m, and ABE8.20m. The splice acceptor target that was edited for exon skipping was A3 (FIG. 17A, FIG. 17C). A transfection experiment was performed in HEK293T cells with 750 ng of ABE plasmid and 250 ng of gRNA plasmid or pmaxGFP. HEK293 cells were plated in a 48-well (30,000 cells/well). The next day, 750 ng base editor plasmid and 250 ng gRNA plasmid or pmaxGFP were transfected with Lipefectamine 2000. Quick extract was harvested 3 days after transfection, the region around the splice acceptor amplified by PCR, amplicons were subjected to deep sequencing, and data were analyzed using CRISPResso software to determine the proportion of editing at each position. Results showed that after three days, ABE8e and ABE8.17m, when paired with g02, showed the most efficient editing at this position (FIG. 17B, FIG. 17D). While all ABEs tested showed high levels of editing in at least one of the adenines in the editing window (data not shown), only the 8th generation editors (ABE8e, ABE8.8m, ABE8.13m, ABE8.17m, and ABE8.20m) with broadened editing windows were able to efficiently edit the adenine of the splice acceptor (A3). The editing efficiency for the top two conditions, 52.37% for ABE8e and g02 and 51.11% for ABE8.17m with g02, was an order of magnitude higher that that observed when a similar experiment was conducted with a panel of CBEs and the one gRNA capable of targeting the exon 45 splice acceptor (FIG. 17B, FIG. 17D). As a result, these two high-performing ABE conditions were chosen to study the effect of base editing on exon skipping.

This experiment was repeated to examine bystander editing of neighboring A's with ABE8e (FIG. 17E) and ABE.17m (FIG. 17F). For this application, bystander edits should not interfere with splice site disruption or coding sequence. Next, the purity of products formed with ABE8e and ABE8.17m paired with g02 was examined (FIG. 17G). The ABEs used in these experiments are included in the fusion proteins of SEQ ID NOs: 27-34. ABE8e enabled highly efficient base editing of the hDMD exon 45 splice acceptor in HEK293T cells.

### Example 4

### Editing and Differentiation of Δ44 iPSCs for Assessment of Exon Skipping

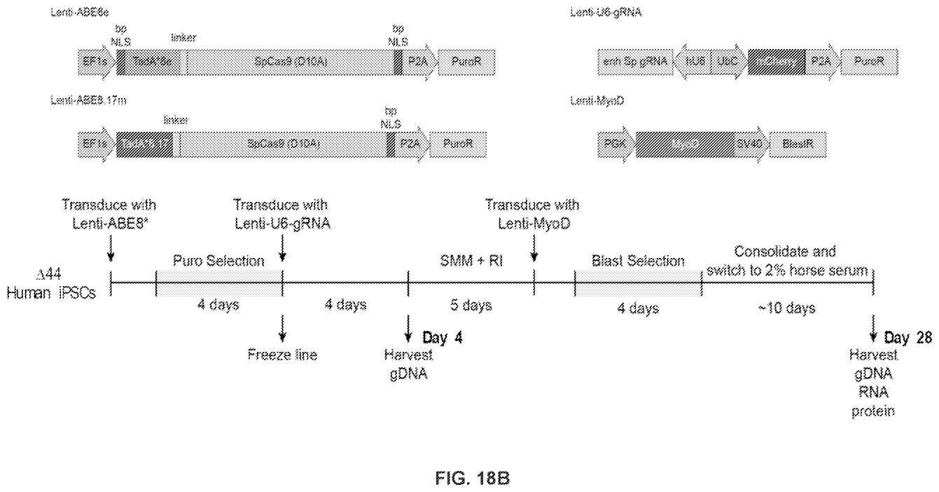

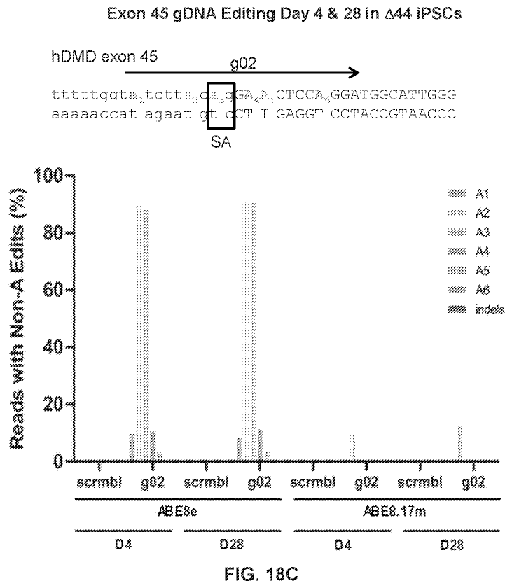

A human iPSC cell line with exon 44 deleted from the dystrophin gene was created, referred to as Δ44 (FIG. 18A). SpCas9 and two gRNAs were used to excise exon 44, which shifts the dystrophin gene out of frame. The reading frame in Δ44 cells can be restored by skipping exon 45. Shown in FIG. 18B is a schematic of the lentiviral constructs used for iPSC editing and differentiation. Δ44 iPSCs were transduced with either ABE8e or ABE8.17m and selected to create stable lines. At day 0, either g02 or a scrambled control were transduced, but not selected on. To achieve dystrophin expression, ABE+gRNA cells were cultured in skeletal muscle media (SMM), transduced with a lentiviral construct with constitutive MyoD cDNA, and further differentiated in low serum conditions. As shown in FIG. 18C, ABE8e and g02 exhibited 88.6% splice acceptor base editing in Δ44 iPSCs 4 days post-gRNA transduction (no selection on gRNA lenti). There were minimal increases in DNA editing during the MyoD differentiation. ABE8e enabled highly efficient base editing of the hDMD exon 45 splice acceptor in iPSC cells.

### Example 5

### Editing Exon 45 Splice Acceptor Causes Exon Skipping and Protein Restoration

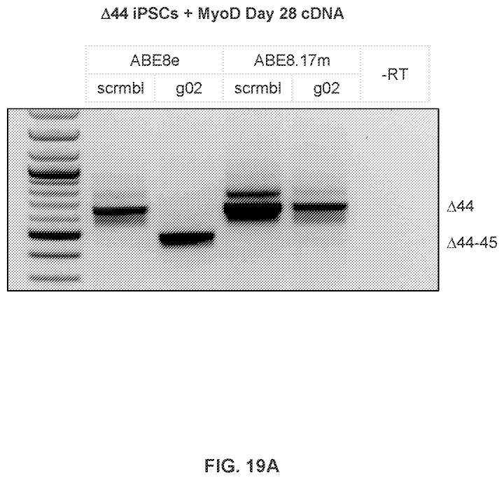

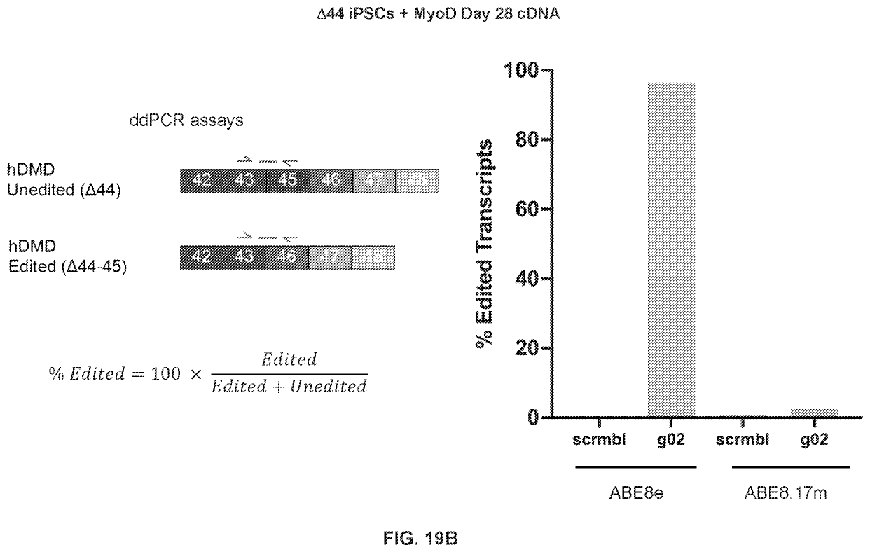

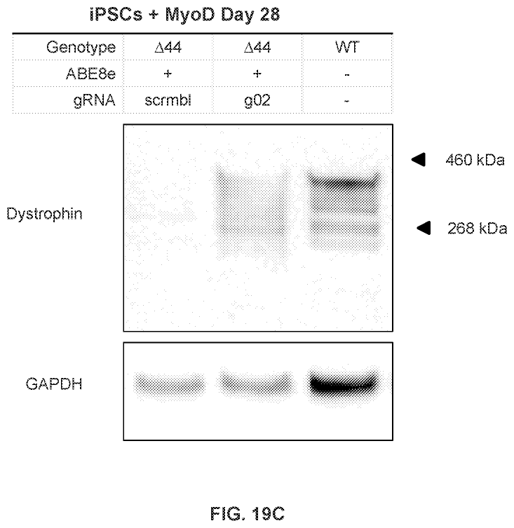

The editing of exon 45 splice acceptor with ABE8e or ABE8.17m in Δ44 iPSC cells was examined. cDNA extracted on Day 28 from the Δ44 iPSCs+ABE+gRNA+MyoD differentiation cells was amplified by RT-PCR (FIG. 19A). The high level of exon 45 splice acceptor base editing observed with ABE8e+g02 corresponds with a strong shift towards transcripts skipping exon 45. The cDNA from Day 28 was then quantified by ddPCR (FIG. 19B), showing that ABE8e+g02 exhibited 96.6% exon 45 skipping. Restoration of dystrophin expression was examined via Westem Blot analysis (FIG. 19C), showing that ABE8e+g02 rescued dystrophin protein expression that was not present in unedited Δ44 iPSCs. Myogenic differentiation of base edited Δ44 iPSCs demonstrated exon skipping after splice site editing, which lead to dystrophin protein restoration.

gRNA-dependent DNA off-target activity will be predicted using CHANGE-seq analysis. Any off-target RNA editing will be analyzed through RNA-seq, and splicing outcomes will be identified and quantified. Split-intein AAV-ABE8e will be used to edit new hDMDΔ44/mdx mice to assess the functional benefit of splice acceptor editing and investigate the editing products.

### Example 6

### Base Editing for Skipping Exon 45

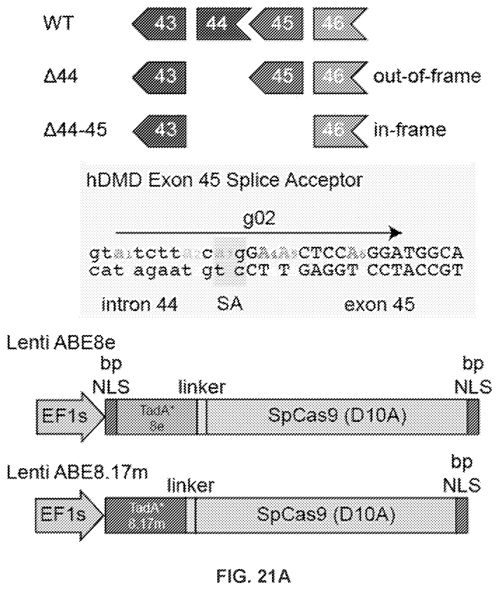

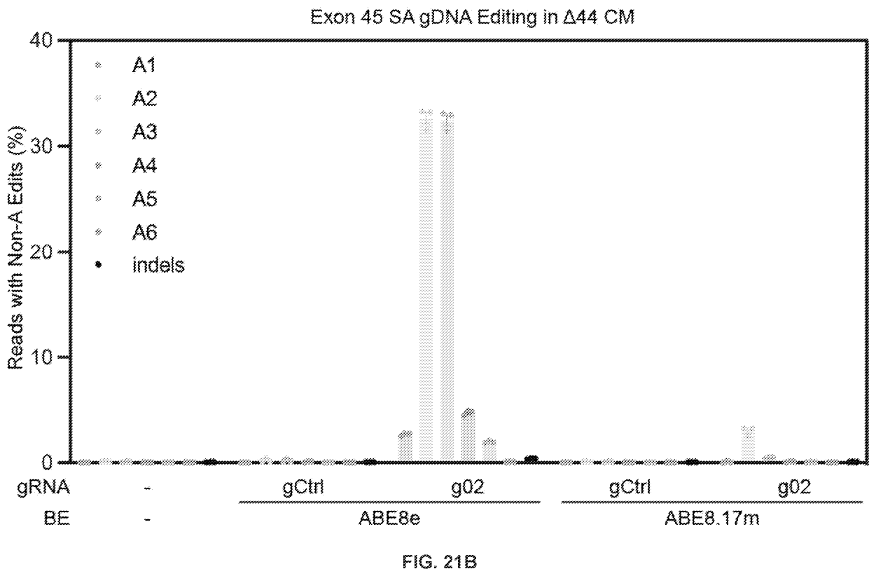

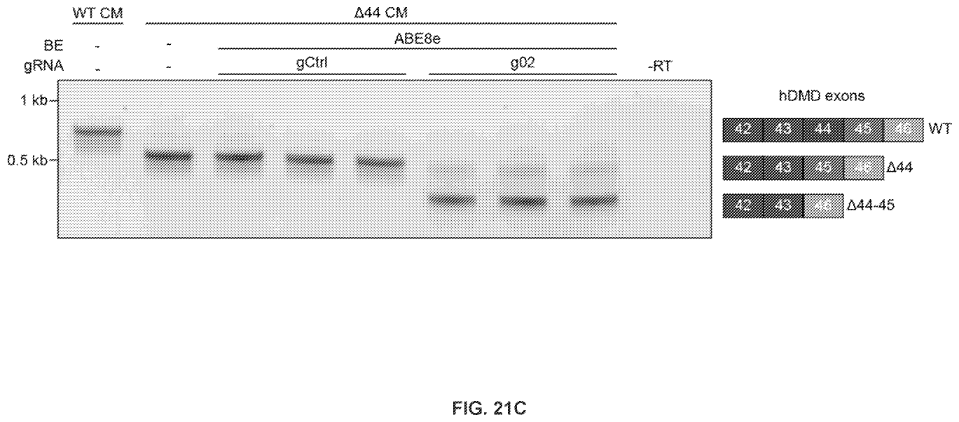

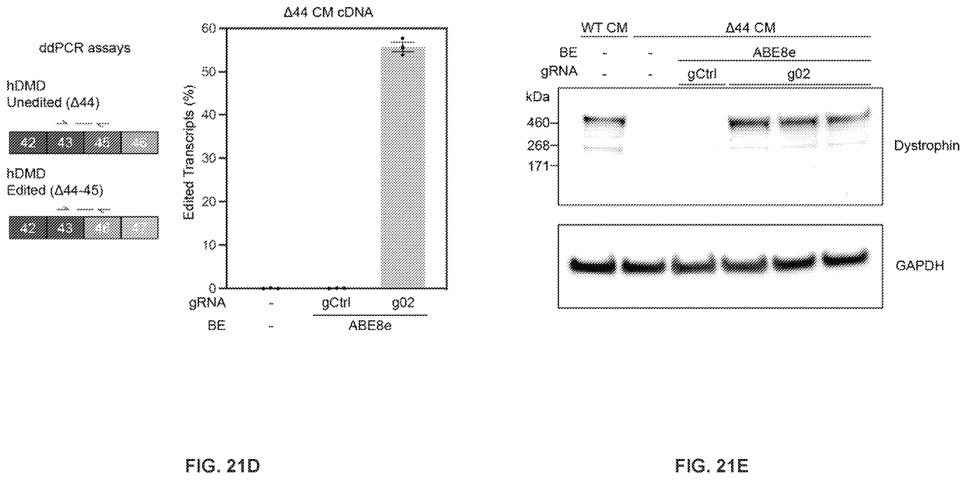

Dystrophin is lowly expressed in non-muscle tissues, so iPSC-derived cardiomyocytes (CM) were applied as an in vitro model to study how base editing the exon 45 splice acceptor impacts DMD splicing. To model the transcript and protein restoration expected when correcting a DMD patient mutation. SpCas9 and two gRNAs were used to excise exon 44 from a male wild-type iPS cell line, and an edited Δ44 clone was then selected. When exon 45 is skipped in this line with a DMD genotype, the reading frame should be restored, resulting in internally truncated but functional dystrophin protein (FIG. 21A). Wild-type and Δ44 iPSCs were differentiated into CMs through an 11-day small molecule protocol, followed by 4 days of selection in glucose-free conditions. On day 16, cells were replated and transduced with two lentiviruses, one containing the ABE (either ABE8e or ABE8.17m) and one supplying the U6-gRNA (either g02 targeting the exon 45 splice acceptor or a non-targeting control) (FIG. 21A). Five days after transduction, cells were harvested without selecting for lentiviral transduction, and RNA and protein were isolated. Deep sequencing of the gDNA showed that ABE8e enabled 32.47% conversion of the splice acceptor adenine, only when paired with the targeting gRNA (FIG. 21B). ABE8e is an editor with a broadened window, which is consistent with the observation that neighboring A's were also edited, the most notable being A2. Because A1. A2, and A3 are intronic and A4, A5, and A6 are within the exon that should be skipped, it was not anticipated that these bystander edits would have deleterious effects. Notably, ABE8.17m performed much more poorly in the CMs, compared to both the HEK293T transfection (FIG. 21B) and ABE8e in the CMs. This may be due to the removal of the N-terminal bipartite NLS from this construct compared to earlier versions, resulting in lower levels of nuclear expression.

Endpoint RT-PCR with primers in exons 42 and 46 demonstrated a clear pattern of exon skipping in the ABE8e+g02 samples (FIG. 21C). This exon skipping was quantified by ddPCR, with unedited transcripts measured by a primer probe set spanning the exon 43-45 junction (cells are Δ44), and edited transcripts by the exon 43-46 junction. The fraction of edited transcripts was calculated by dividing the edited concentration by the sum of edited and unedited transcripts. ABE8e+g02 forced exon 45 skipping in 55.72% of transcripts (FIG. 21D). This editing rate at the RNA level was higher than the 32.47% observed at the DNA level. This was likely due to stabilization of DMD transcripts by reading frame restoration amplifying the effect, and indeed, transcript levels in edited CMs were observed to be higher than the Δ44 control by ddPCR (data not shown). The high levels of exon 45 skipping observed translated to restoration of dystrophin protein comparable to wild-type levels (FIG. 21E).

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A CRISPR/Cas-based base editing system for altering an RNA splice site encoded in the genomic DNA of a subject, the CRISPR/Cas-based base editing system comprising a fusion protein and at least one guide RNA (gRNA), wherein the fusion protein comprises a Cas protein and a base-editing domain, and wherein the at least one gRNA targets a sequence comprising at least one of SEQ ID NOs: 21-23 or 43 or a complement or a fragment thereof and/or the gRNA comprises a sequence selected from SEQ ID NOs: 24-26 or 44 or a complement or a fragment thereof.

Clause 2. A CRISPR/Cas-based base editing system for altering an RNA splice site encoded in the genomic DNA of a subject, the CRISPR/Cas-based base editing system comprising a fusion protein and at least one guide RNA (gRNA), wherein the fusion protein comprises a Cas protein and a base-editing domain, and wherein the base-editing domain comprises a polypeptide selected from SEQ ID NOs: 45-52 and/or is encoded by a polynucleotide comprising a sequence selected from SEQ ID NOs: 53-60.

Clause 3. The CRISPR/Cas-based base editing system of clause 2, wherein the fusion protein comprises a polypeptide selected from SEQ ID NOs: 27-34 and/or is encoded by a polynucleotide comprising a sequence selected from SEQ ID NOs: 35-42.

Clause 4. The CRISPR/Cas-based base editing system of any one of clauses 1-3, wherein altering the RNA splice site encoded in the genomic DNA results in exclusion or inclusion of at least one exon sequence in an RNA transcript.

Clause 5. A CRISPR/Cas-based base editing system for restoring dystrophin function in a subject, the CRISPR/Cas-based base editing system comprising a fusion protein and at least one guide RNA (gRNA), wherein the fusion protein comprises a Cas protein and a base-editing domain, wherein the at least one gRNA targets a sequence comprising at least one of SEQ ID NOs: 21-23 or 43 or a complement or a fragment thereof and/or the gRNA comprises a sequence selected from SEQ ID NOs: 24-26 or 44 or a complement or a fragment thereof.

Clause 6. A CRISPR/Cas-based base editing system for restoring dystrophin function in a subject, the CRISPR/Cas-based base editing system comprising a fusion protein and at least one guide RNA (gRNA), wherein the fusion protein comprises a Cas protein and a base-editing domain, and wherein base-editing domain comprises a polypeptide selected from SEQ ID NOs: 45-52 and/or is encoded by a polynucleotide comprising a sequence selected from SEQ ID NOs: 53-60.

Clause 7. The CRISPR/Cas-based base editing system of clause 6, wherein the fusion protein comprises a polypeptide selected from SEQ ID NOs: 27-34 and/or is encoded by a polynucleotide comprising a sequence selected from SEQ ID NOs: 35-42.

Clause 8. The CRISPR/Cas-based base editing system of any one of clauses 5-7, wherein the subject has a mutated dystrophin gene, and wherein the at least one guide RNA (gRNA) targets an RNA splice site in the mutated dystrophin gene of the subject.

Clause 9. The CRISPR/Cas-based base editing system of clause 8, wherein administration of the CRISPR/Cas-based base editing system to the subject results in at least one exon sequence being excluded or included in an RNA transcript of the dystrophin gene of the subject and the reading frame of dystrophin gene in the subject being restored.

Clause 10. The CRISPR/Cas-based base editing system any one of clauses 1-9, wherein the Cas protein comprises a Cas9, and wherein the Cas9 comprises at least one amino acid mutation which eliminates the nuclease activity of Cas9.

Clause 11. The CRISPR/Cas-based base editing system of clause 10, wherein the at least one amino acid mutation is at least one of D10A, H840A, or a combination thereof, in the amino acid sequence corresponding to SEQ ID NO: 2 or 3.

Clause 12. The CRISPR/Cas-based base editing system of any one of clauses 1-11, wherein the Cas protein is a Streptococcus pyogenes Cas9 protein or a Staphylococcus aureus Cas9 protein.

Clause 13. The CRISPR/Cas-based base editing system of any one of clauses 1-12, wherein the Cas protein comprises an amino acid sequence of SEQ ID NO: 4 or 5.

Clause 14. The CRISPR/Cas-based base editing system of any one of clauses 1-13, wherein the base-editing domain further comprises (i) a cytidine deaminase domain and (ii) at least one uracil glycosylase inhibitor (UGI) domain.

Clause 15. The CRISPR/Cas-based base editing system of clause 14, wherein the cytidine deaminase domain comprises an apolipoprotein B mRNA-editing enzyme, catalytic polypeptide-like (APOBEC) deaminase.

Clause 16. The CRISPR/Cas-based base editing system of clause 14 or 15, wherein the cytidine deaminase domain comprises an APOBEC 1 deaminase.

Clause 17. The CRISPR/Cas-based base editing system of clause 16, wherein the cytidine deaminase domain comprises a rat APOBEC 1 deaminase.

Clause 18. The CRISPR/Cas-based base editing system of any one of clauses 14-17, wherein the at least one UGI domain comprises a domain capable of inhibiting UDG activity.

Clause 19. The CRISPR/Cas-based base editing system of clause 18, wherein the at least one UGI domain comprises the amino acid sequence of SEQ ID NO: 20 or an amino acid sequence encoded by the polynucleotide sequence of SEQ ID NO: 6 or SEQ ID NO: 18.

Clause 20. The CRISPR/Cas-based base editing system of any one of clauses 14-19, wherein the base-editing domain comprises one UGI domain or two UGI domains.

Clause 21. The CRISPR/Cas-based base editing system of any one of clauses 1-20, wherein the fusion protein comprises the structure: NH₂-[ABE]-[Cas protein]-COOH, and wherein each instance of “-” comprises an optional linker.

Clause 22. The CRISPR/Cas-based base editing system of any one of clauses 1-20, wherein the fusion protein comprises the structure: NH₂-[Cas protein]-[ABE]-COOH, and wherein each instance of “-” comprises an optional linker.

Clause 23. The CRISPR/Cas-based base editing system of any one of clauses 1-22, wherein the fusion protein further comprises a nuclear localization sequence (NLS).

Clause 24. An isolated polynucleotide encoding the CRISPR/Cas-based base editing system of any one of clauses 1-23.

Clause 25. The isolated polynucleotide of clause 24, wherein the polynucleotide comprises a first polynucleotide encoding the fusion protein and a second polynucleotide encoding the gRNA.

Clause 26. A vector comprising the isolated polynucleotide of clause 24 or 25.

Clause 27. The vector of clause 26, wherein the vector comprises a heterologous promoter driving expression of the isolated polynucleotide.

Clause 28. A cell comprising the isolated polynucleotide of clause 24 or 25 or the vector of clause 26 or 27.

Clause 29. A composition for restoring dystrophin function in a cell having a mutant dystrophin gene, the composition comprising the CRISPR/Cas-based base editing system of any one of clauses 1-23.

Clause 30. A kit comprising the CRISPR/Cas-based base editing system of any one of clauses 1-23, the isolated polynucleotide of clause 24 or 25, the vector of clause 26 or 27, the cell of clause 28, or the composition of clause 29.

Clause 31. A method for restoring dystrophin function in a cell or a subject having a mutant dystrophin gene, the method comprising contacting the cell or the subject with the CRISPR/Cas-based base editing system of any one of clauses 1-23.

Clause 32. The method of clause 31, wherein an “AG” splice acceptor in exon 45 of the mutant dystrophin gene is converted to an “GG” sequence and the dystrophin function is restored by exon 45 skipping.

Clause 33. The method of clause 31 or 32, wherein the subject is suffering from Duchenne Muscular Dystrophy.

SEQUENCES
Target sequence of the Exon 45 gRNA (SEQ ID NO: 1)
gttcctgtaagataccaaaa
Streptococcus pyogenes Cas 9 (SEQ ID NO: 2)
MDKKYSIGLDIGTNSVGWAVITDEYKVPSKKFKVLGNTDRHSIKKNLIGALLFDSGETAEATRLKRTA
RRRYTREKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNILAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
S. aureus Cas9 molecule (SEQ ID NO: 3)
MKRNYILGLDIGITSVGYGIIDYETRDVIDAGVRLFKEANVENNEGRRSKRGARRLKRRRRHRIQRVK
KLLFDYNLLTDHSELSGINPYEARVKGLSQKLSEEEFSAALLHLAKRRGVHNVNEVEEDTGNELSTKE
QISRNSKALEEKYVAELQLERLKKDGEVRGSINRFKTSDYVKEAKQLLKVQKAYHQLDQSFIDTYIDL
LETRRTYYEGPGEGSPFGWKDIKEWYEMLMGHCTYFPEELRSVKYAYNADLYNALNDLNNLVITRDEN
EKLEYYEKFQIIENVFKQKKKPTLKQIAKEILVNEEDIKGYRVTSTGKPEFTNLKVYHDIKDITARKE
IIENAELLDQIAKILTIYQSSEDIQEELTNLNSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDEKDYKYSHRVDKKPNRELINDTLYSTRKDDKGNTLIVNNLNGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
Streptococcus pyogenes Cas 9 (with D10A) (SEQ ID NO: 4)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKERGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLEKTNRKVTVKQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Streptococcus pyogenes Cas 9 (with D10A, H849A) (SEQ ID NO: 5)
MDKKYSIGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSKDTYD
DDLDNILAQIGDQYADLFLAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLEKTNRKVTVKQLKEDYFKKIECFDSVEISGVEDRENASLGTYHDLLKIIKDKDFLDNEEN
EDILEDIVLTLTLFEDREMIEERLKTYAHLEDDKVMKQLKRRRYTGWGRLSRKLINGIRDKQSGKTIL
DELKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDAIVPQSFLKDDSIDNKVLTRSDKNRGKSDNVPSEEVVKKMKNYWR
QLLNAKLITQRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
Polynucleotide encoding UGI-1 (SEQ ID NO: 6)
actaatctgagcgacatcattgagaaggagactgggaaacagctggtcattcaggagtccatcctgat
gctgcctgaggaggtggaggaagtgatcggcaacaagccagagtctgacatcctggtgcacaccgcct
acgacgagtccacagatgagaatgtgatgctgctgacctctgacgcccccgagtataagccttgggcc
ctggtcatccaggattctaacggcgagaataagatcaagatgctg
pCMV_BE4max Sequence (SEQ ID NO: 7)
atatgccaagtacgccccctattgacgtcaatgacggtaaatggcccgcctggcattatgcccagtac
atgaccttatgggactttcctacttggcagtacatctacgtattagtcatcgctattaccatggtgat
gcggttttggcagtacatcaatgggcgtggatagcggtttgactcacggggatttccaagtctccacc
ccattgacgtcaatgggagtttgttttggcaccaaaatcaacgggactttccaaaatgtcgtaacaac
tccgccccattgacgcaaatgggcggtaggcgtgtacggtgggaggtctatataagcagagctggttt
agtgaaccgtcagatccgctagagatccgcggccgctaatacgactcactatagggagagccgccacc
atgaaacggacagccgacggaagcgagttcgagtcaccaaagaagaagcggaaagtctcctcagagac
tgggcctgtcgccgtcgatccaaccctgcgccgccggattgaacctcacgagtttgaagtgttctttg
acccccgggagctgagaaaggagacatgcctgctgtacgagatcaactggggaggcaggcactccatc
tggaggcacacctctcagaacacaaataagcacgtggaggtgaacttcatcgagaagtttaccacaga
gcggtacttctgccccaataccagatgtagcatcacatggtttctgagctggtccccttgcggagagt
gtagcagggccatcaccgagttcctgtccagatatccacacgtgacactgtttatctacatcgccagg
ctgtatcaccacgcagacccaaggaataggcagggcctgcgcgatctgatcagctccggcgtgaccat
ccagatcatgacagagcaggagtccggctactgctggcggaacttcgtgaattattctcctagcaacg
aggcccactggcctaggtacccacacctgtgggtgcgcctgtacgtgctggagctgtattgcatcatc
ctgggcctgcccccttgtctgaatatcctgcggagaaagcagccccagctgaccttctttacaatcgc
cctgcagtcttgtcactatcagaggctgccaccccacatcctgtgggccacaggcctgaagtctggag
gatctagcggaggatcctctggcagcgagacaccaggaacaagcgagtcagcaacaccagagagcagt
ggcggcagcagcggcggcagcgacaagaagtacagcatcggcctggccatcggcaccaactctgtggg
ctgggccgtgatcaccgacgagtacaaggtgcccagcaagaaattcaaggtgctgggcaacaccgacc
ggcacagcatcaagaagaacctgatcggagccctgctgttcgacagcggcgaaacagccgaggccacc
cggctgaagagaaccgccagaagaagatacaccagacggaagaaccggatctgctatctgcaagagat
cttcagcaacgagatggccaaggtggacgacagcttcttccacagactggaagagtccttcctggtgg
aagaggataagaagcacgagcggcaccccatcttcggcaacatcgtggacgaggtggcctaccacgag
aagtaccccaccatctaccacctgagaaagaaactggtggacagcaccgacaaggccgacctgcggct
gatctatctggccctggcccacatgatcaagttccggggccacttcctgatcgagggcgacctgaacc
ccgacaacagcgacgtggacaagctgttcatccagctggtgcagacctacaaccagctgttcgaggaa
aaccccatcaacgccagcggcgtggacgccaaggccatcctgtctgccagactgagcaagagcagacg
gctggaaaatctgatcgcccagctgcccggcgagaagaagaatggcctgttcggaaacctgattgccc
tgagcctgggcctgacccccaacttcaagagcaacttcgacctggccgaggatgccaaactgcagctg
agcaaggacacctacgacgacgacctggacaacctgctggcccagatcggcgaccagtacgccgacct
gtttctggccgccaagaacctgtccgacgccatcctgctgagcgacatcctgagagtgaacaccgaga
tcaccaaggcccccctgagcgcctctatgatcaagagatacgacgagcaccaccaggacctgaccctg
ctgaaagctctcgtgcggcagcagctgcctgagaagtacaaagagattttcttcgaccagagcaagaa
cggctacgccggctacattgacggcggagccagccaggaagagttctacaagttcatcaagcccatcc
tggaaaagatggacggcaccgaggaactgctcgtgaagctgaacagagaggacctgctgcggaagcag
cggaccttcgacaacggcagcatcccccaccagatccacctgggagagctgcacgccattctgcggcg
gcaggaagatttttacccattcctgaaggacaaccgggaaaagatcgagaagatcctgaccttccgca
tcccctactacgtgggccctctggccaggggaaacagcagattcgcctggatgaccagaaagagcgag
gaaaccatcaccccctggaacttcgaggaagtggtggacaagggcgcttccgcccagagcttcatcga
gcggatgaccaacttcgataagaacctgcccaacgagaaggtgctgcccaagcacagcctgctgtacg
agtacttcaccgtgtataacgagctgaccaaagtgaaatacgtgaccgagggaatgagaaagcccgcc
ttcctgagcggcgagcagaaaaaggccatcgtggacctgctgttcaagaccaaccggaaagtgaccgt
gaagcagctgaaagaggactacttcaagaaaatcgagtgcttcgactccgtggaaatctccggcgtgg
aagatcggttcaacgcctccctgggcacataccacgatctgctgaaaattatcaaggacaaggacttc
ctggacaatgaggaaaacgaggacattctggaagatatcgtgctgaccctgacactgtttgaggacag
agagatgatcgaggaacggctgaaaacctatgcccacctgttcgacgacaaagtgatgaagcagctga
agcggcggagatacaccggctggggcaggctgagccggaagctgatcaacggcatccgggacaagcag
tccggcaagacaatcctggatttcctgaagtccgacggcttcgccaacagaaacttcatgcagctgat
ccacgacgacagcctgacctttaaagaggacatccagaaagcccaggtgtccggccagggcgatagcc
tgcacgagcacattgccaatctggccggcagccccgccattaagaagggcatcctgcagacagtgaag
gtggtggacgagctcgtgaaagtgatgggccggcacaagcccgagaacatcgtgatcgaaatggccag
agagaaccagaccacccagaagggacagaagaacagccgcgagagaatgaagcggatcgaagagggca
tcaaagagctgggcagccagatcctgaaagaacaccccgtggaaaacacccagctgcagaacgagaag
ctgtacctgtactacctgcagaatgggcgggatatgtacgtggaccaggaactggacatcaaccggct
gtccgactacgatgtggaccatatcgtgcctcagagctttctgaaggacgactccatcgacaacaagg
tgctgaccagaagcgacaagaaccggggcaagagcgacaacgtgccctccgaagaggtcgtgaagaag
atgaagaactactggcggcagctgctgaacgccaagctgattacccagagaaagttcgacaatctgac
caaggccgagagaggcggcctgagcgaactggataaggccggcttcatcaagagacagctggtggaaa
cccggcagatcacaaagcacgtggcacagatcctggactcccggatgaacactaagtacgacgagaat
gacaagctgatccgggaagtgaaagtgatcaccctgaagtccaagctggtgtccgatttccggaagga
tttccagttttacaaagtgcgcgagatcaacaactaccaccacgcccacgacgcctacctgaacgccg
tcgtgggaaccgccctgatcaaaaagtaccctaagctggaaagcgagttcgtgtacggcgactacaag
gtgtacgacgtgcggaagatgatcgccaagagcgagcaggaaatcggcaaggctaccgccaagtactt
cttctacagcaacatcatgaactttttcaagaccgagattaccctggccaacggcgagatccggaagc
ggcctctgatcgagacaaacggcgaaaccggggagatcgtgtgggataagggccgggattttgccacc
gtgcggaaagtgctgagcatgccccaagtgaatatcgtgaaaaagaccgaggtgcagacaggcggctt
cagcaaagagtctatcctgcccaagaggaacagcgataagctgatcgccagaaagaaggactgggacc
ctaagaagtacggcggcttcgacagccccaccgtggcctattctgtgctggtggtggccaaagtggaa
aagggcaagtccaagaaactgaagagtgtgaaagagctgctggggatcaccatcatggaaagaagcag
cttcgagaagaatcccatcgactttctggaagccaagggctacaaagaagtgaaaaaggacctgatca
tcaagctgcctaagtactccctgttcgagctggaaaacggccggaagagaatgctggcctctgccggc
gaactgcagaagggaaacgaactggccctgccctccaaatatgtgaacttcctgtacctggccagcca
ctatgagaagctgaagggctcccccgaggataatgagcagaaacagctgtttgtggaacagcacaagc
actacctggacgagatcatcgagcagatcagcgagttctccaagagagtgatcctggccgacgctaat
ctggacaaagtgctgtccgcctacaacaagcaccgggataagcccatcagagagcaggccgagaatat
catccacctgtttaccctgaccaatctgggagcccctgccgccttcaagtactttgacaccaccatcg
accggaagaggtacaccagcaccaaagaggtgctggacgccaccctgatccaccagagcatcaccggc
ctgtacgagacacggatcgacctgtctcagctgggaggtgacagcggcgggagcggcgggagcggggg
gagcactaatctgagcgacatcattgagaaggagactgggaaacagctggtcattcaggagtccatcc
tgatgctgcctgaggaggtggaggaagtgatcggcaacaagccagagtctgacatcctggtgcacacc
gcctacgacgagtccacagatgagaatgtgatgctgctgacctctgacgcccccgagtataagccttg
ggccctggtcatccaggattctaacggcgagaataagatcaagatgctgagcggaggatccggaggat
ctggaggcagcaccaacctgtctgacatcatcgagaaggagacaggcaagcagctggtcatccaggag
agcatcctgatgctgcccgaagaagtcgaagaagtgatcggaaacaagcctgagagcgatatcctggt
ccataccgcctacgacgagagtaccgacgaaaatgtgatgctgctgacatccgacgccccagagtata
agccctgggctctggtcatccaggattccaacggagagaacaaaatcaaaatgctgtctggcggctca
aaaagaaccgccgacggcagcgaattcgagcccaagaagaagaggaaagtctaaccggtcatcatcac
catcaccattgagtttaaacccgctgatcagcctcgactgtgccttctagttgccagccatctgttgt
ttgcccctcccccgtgccttccttgaccctggaaggtgccactcccactgtcctttcctaataaaatg
aggaaattgcatcgcattgtctgagtaggtgtcattctattctggggggtggggtggggcaggacagc
aagggggaggattgggaagacaatagcaggcatgctggggatgcggtgggctctatggcttctgaggc
ggaaagaaccagctggggctcgataccgtcgacctctagctagagcttggcgtaatcatggtcatagc
tgtttcctgtgtgaaattgttatccgctcacaattccacacaacatacgagccggaagcataaagtgt
aaagcctagggtgcctaatgagtgagctaactcacattaattgcgttgcgctcactgcccgctttcca
gtcgggaaacctgtcgtgccagctgcattaatgaatcggccaacgcgcggggagaggcggtttgcgta
ttgggcgctcttccgcttcctcgctcactgactcgctgcgctcggtcgttcggctgcggcgagcggta
tcagctcactcaaaggcggtaatacggttatccacagaatcaggggataacgcaggaaagaacatgtg
agcaaaaggccagcaaaaggccaggaaccgtaaaaaggccgcgttgctggcgtttttccataggctcc
gcccccctgacgagcatcacaaaaatcgacgctcaagtcagaggtggcgaaacccgacaggactataa
agataccaggcgtttccccctggaagctccctcgtgcgctctcctgttccgaccctgccgcttaccgg
atacctgtccgcctttctcccttcgggaagcgtggcgctttctcatagctcacgctgtaggtatctca
gttcggtgtaggtcgttcgctccaagctgggctgtgtgcacgaaccccccgttcagcccgaccgctgc
gccttatccggtaactatcgtcttgagtccaacccggtaagacacgacttatcgccactggcagcagc
cactggtaacaggattagcagagcgaggtatgtaggcggtgctacagagttcttgaagtggtggccta
actacggctacactagaagaacagtatttggtatctgcgctctgctgaagccagttaccttcggaaaa
agagttggtagctcttgatccggcaaacaaaccaccgctggtagcggtggtttttttgtttgcaagca
gcagattacgcgcagaaaaaaaggatctcaagaagatcctttgatcttttctacggggtctgacactc
agtggaacgaaaactcacgttaagggattttggtcatgagattatcaaaaaggatcttcacctagatc
cttttaaattaaaaatgaagttttaaatcaatctaaagtatatatgagtaaacttggtctgacagtta
ccaatgcttaatcagtgaggcacctatctcagcgatctgtctatttcgttcatccatagttgcctgac
tccccgtcgtgtagataactacgatacgggagggcttaccatctggccccagtgctgcaatgataccg
cgagacccacgctcaccggctccagatttatcagcaataaaccagccagccggaagggccgagcgcag
aagtggtcctgcaactttatccgcctccatccagtctattaattgttgccgggaagctagagtaagta
gttcgccagttaatagtttgcgcaacgttgttgccattgctacaggcatcgtggtgtcacgctcgtcg
tttggtatggcttcattcagctccggttcccaacgatcaaggcgagttacatgatcccccatgttgtg
caaaaaagcggttagctccttcggtcctccgatcgttgtcagaagtaagttggccgcagtgttatcac
tcatggttatggcagcactgcataattctcttactgtcatgccatccgtaagatgcttttctgtgact
ggtgagtactcaaccaagtcattctgagaatagtgtatgcggcgaccgagttgctcttgcccggcgtc
aatacgggataataccgcgccacatagcagaactttaaaagtgctcatcattggaaaacgttcttcgg
ggcgaaaactctcaaggatcttaccgctgttgagatccagttcgatgtaacccactcgtgcacccaac
tgatcttcagcatcttttactttcaccagcgtttctgggtgagcaaaaacaggaaggcaaaatgccgc
aaaaaagggaataagggcgacacggaaatgttgaatactcatactcttcctttttcaatattattgaa
gcatttatcagggttattgtctcatgagcggatacatatttgaatgtatttagaaaaataaacaaata
ggggttccgcgcacatttccccgaaaagtgccacctgacgtcgacggatcgggagatcgatctcccga
tcccctagggtcgactctcagtacaatctgctctgatgccgcatagttaagccagtatctgctccctg
cttgtgtgttggaggtcgctgagtagtgcgcgagcaaaatttaagctacaacaaggcaaggcttgacc
gacaattgcatgaagaatctgcttagggttaggcgttttgcgctgcttcgcgatgtacgggccagata
tacgcgttgacattgattattgactagttattaatagtaatcaattacggggtcattagttcatagcc
catatatggagttccgcgttacataacttacggtaaatggcccgcctggctgaccgcccaacgacccc
cgcccattgacgtcaataatgacgtatgttcccatagtaacgccaatagggactttccattgacgtca
atgggtggagtatttacggtaaactgcccacttggcagtacatcaagtgtatc
pCMV_AncBE4max Sequence (SEQ ID NO: 8)
atatgccaagtacgccccctattgacgtcaatgacggtaaatggcccgcctggcattatgcccagtac
atgaccttatgggactttcctacttggcagtacatctacgtattagtcatcgctattaccatggtgat
gcggttttggcagtacatcaatgggcgtggatagcggtttgactcacggggatttccaagtctccacc
ccattgacgtcaatgggagtttgttttggcaccaaaatcaacgggactttccaaaatgtcgtaacaac
tccgccccattgacgcaaatgggggtaggcgtgtacggtgggaggtctatataagcagagctggttt
agtgaaccgtcagatccgctagagatccgcggccgctaatacgactcactatagggagagccgccacc
atgaaacggacagccgacggaagcgagttcgagt caccaaagaagaagcggaaagtcagcagtgaaac
cggaccagtggcagtggacccaaccctgaggagacggattgagccccatgaatttgaagtgttctttg
acccaagggagctgaggaaggagacatgcctgctgtacgagatcaagtggggcacaagccacaagatc
tggcgccacagctccaagaacaccacaaagcacgtggaagtgaatttcatcgagaagtttacctccga
gcggcacttctgcccctctaccagctgttccatcacatggtttctgtcttggagcccttgcggcgagt
gttccaaggccatcaccgagttcctgtctcagcaccctaacgtgaccctggtcatctacgtggcccgg
ctgtatcaccacatggaccagcagaacaggcagggcctgcgcgatctggtgaattctggcgtgaccat
ccagatcatgacagccccagagtacgactattgctggcggaacttcgtgaattatccacctggcaagg
aggcacactggccaagatacccacccctgtggatgaagctgtatgcactggagctgcacgcaggaatc
ctgggcctgcctccatgtctgaatatcctgcggagaaagcagccccagctgacatttttcaccattgc
tctgcagtcttgtcactatcagcggctgcctcctcatattctgtgggctacaggcctgaagtctggag
gatctagcggaggatcctctggcagcgagacaccaggaacaagcgagtcagcaacaccagagagcagt
ggcggcagcagcggcggcagcgacaagaagtacagcatcggcctggccatcggcaccaactctgtggg
ctgggccgtgatcaccgacgagtacaaggtgcccagcaagaaattcaaggtgctgggcaacaccgacc
ggcacagcatcaagaagaacctgatcggagccctgctgttcgacagcggcgaaacagccgaggccacc
cggctgaagagaaccgccagaagaagatacaccagacggaagaaccggatctgctatctgcaagagat
cttcagcaacgagatggccaaggtggacgacagcttcttccacagactggaagagtccttcctggtgg
aagaggataagaagcacgagcggcaccccatcttcggcaacatcgtggacgaggtggcctaccacgag
aagtaccccaccatctaccacctgagaaagaaactggtggacagcaccgacaaggccgacctgcggct
gatctatctggccctggcccacatgatcaagttccggggccacttcctgatcgagggcgacctgaacc
ccgacaacagcgacgtggacaagctgttcatccagctggtgcagacctacaaccagctgttcgaggaa
aaccccatcaacgccagcggcgtggacgccaaggccatcctgtctgccagactgagcaagagcagacg
gctggaaaatctgatcgcccagctgcccggcgagaagaagaatggcctgttcggaaacctgattgccc
tgagcctgggcctgacccccaacttcaagagcaacttcgacctggccgaggatgccaaactgcagctg
agcaaggacacctacgacgacgacctggacaacctgctggcccagatcggcgaccagtacgccgacct
gtttctggccgccaagaacctgtccgacgccatcctgctgagcgacatcctgagagtgaacaccgaga
tcaccaaggcccccctgagcgcctctatgatcaagagatacgacgagcaccaccaggacctgaccctg
ctgaaagctctcgtgcggcagcagctgcctgagaagtacaaagagattttcttcgaccagagcaagaa
cggctacgccggctacattgacggcggagccagccaggaagagttctacaagttcatcaagcccatcc
tggaaaagatggacggcaccgaggaactgctcgtgaagctgaacagagaggacctgctgcggaagcag
cggaccttcgacaacggcagcatcccccaccagatccacctgggagagctgcacgccattctgcggcg
gcaggaagatttttacccattcctgaaggacaaccgggaaaagatcgagaagatcctgaccttccgca
tcccctactacgtgggccctctggccaggggaaacagcagattcgcctggatgaccagaaagagcgag
gaaaccatcaccccctggaacttcgaggaagtggtggacaagggcgcttccgcccagagcttcatcga
gcggatgaccaacttcgataagaacctgcccaacgagaaggtgctgcccaagcacagcctgctgtacg
agtacttcaccgtgtataacgagctgaccaaagtgaaatacgtgaccgagggaatgagaaagcccgcc
ttcctgagcggcgagcagaaaaaggccatcgtggacctgctgttcaagaccaaccggaaagtgaccgt
gaagcagctgaaagaggactacttcaagaaaatcgagtgcttcgactccgtggaaatctccggcgtgg
aagatcggttcaacgcctccctgggcacataccacgatctgctgaaaattatcaaggacaaggacttc
ctggacaatgaggaaaacgaggacattctggaagatatcgtgctgaccctgacactgtttgaggacag
agagatgatcgaggaacggctgaaaacctatgcccacctgttcgacgacaaagtgatgaagcagctga
agcggcggagatacaccggctggggcaggctgagccggaagctgatcaacggcatccgggacaagcag
tccggcaagacaatcctggatttcctgaagtccgacggcttcgccaacagaaacttcatgcagctgat
ccacgacgacagcctgacctttaaagaggacatccagaaagcccaggtgtccggccagggcgatagcc
tgcacgagcacattgccaatctggccggcagccccgccattaagaagggcatcctgcagacagtgaag
gtggtggacgagctcgtgaaagtgatgggccggcacaagcccgagaacatcgtgatcgaaatggccag
agagaaccagaccacccagaagggacagaagaacagccgcgagagaatgaagcggatcgaagagggca
tcaaagagctgggcagccagatcctgaaagaacaccccgtggaaaacacccagctgcagaacgagaag
ctgtacctgtactacctgcagaatgggcgggatatgtacgtggaccaggaactggacatcaaccggct
gtccgactacgatgtggaccatatcgtgcctcagagctttctgaaggacgactccatcgacaacaagg
tgctgaccagaagcgacaagaaccggggcaagagcgacaacgtgccctccgaagaggtcgtgaagaag
atgaagaactactggcggcagctgctgaacgccaagctgattacccagagaaagttcgacaatctgac
caaggccgagagaggcggcctgagcgaactggataaggccggcttcatcaagagacagctggtggaaa
cccggcagatcacaaagcacgtggcacagatcctggactcccggatgaacactaagtacgacgagaat
gacaagctgatccgggaagtgaaagtgatcaccctgaagtccaagctggtgtccgatttccggaagga
tttccagttttacaaagtgcgcgagatcaacaactaccaccacgcccacgacgcctacctaaacgccg
tcgtgggaaccgccctgatcaaaaagtaccctaagctggaaagcgagttcgtgtacggcgactacaag
gtgtacgacgtgcggaagatgatcgccaagagcgagcaggaaatcggcaaggctaccgccaagtactt
cttctacagcaacatcatgaactttttcaagaccgagattaccctggccaacggcgagatccggaagc
ggcctctgatcgagacaaacggcgaaaccggggagatcgtgtgggataagggccgggattttgccacc
gtgcggaaagtgctgagcatgccccaagtgaatatcgtgaaaaagaccgaggtgcagacaggcggctt
cagcaaagagtctatcctgcccaagaggaacagcgataagctgatcgccagaaagaaggactgggacc
ctaagaagtacggcggcttcgacagccccaccgtggcctattctgtgctggtggtggccaaagtggaa
aagggcaagtccaagaaactgaagagtgtgaaagagctgctggggatcaccatcatggaaagaagcag
cttcgagaagaatcccatcgactttctggaagccaagggctacaaagaagtgaaaaaggacctgatca
tcaagctgcctaagtactccctgttcgagctggaaaacggccggaagagaatgctggcctctgccggc
gaactgcagaagggaaacgaactggccctgccctccaaatatgtgaacttcctgtacctggccagcca
ctatgagaagctgaagggctcccccgaggataatgagcagaaacagctgtttgtggaacagcacaagc
actacctggacgagatcatcgagcagatcagcgagttctccaagagagtgatcctggccgacgctaat
ctggacaaagtgctgtccgcctacaacaagcaccgggataagcccatcagagagcaggccgagaatat
catccacctgtttaccctgaccaatctgggagcccctgccgccttcaagtactttgacaccaccatcg
accggaagaggtacaccagcaccaaagaggtgctggacgccaccctgatccaccagagcatcaccggc
ctgtacgagacacggatcgacctgtctcagctgggaggtgacagcggcgggagcggcgggagcggggg
gagcactaatctgagcgacatcattgagaaggagactgggaaacagctggtcattcaggagtccatcc
tgatgctgcctgaggaggtggaggaagtgatcggcaacaagccagagtctgacatcctggtgcacacc
gcctacgacgagtccacagatgagaatgtgatgctgctgacctctgacgcccccgagtataagccttg
ggccctggtcatccaggattctaacggcgagaataagatcaagatgctgagcggaggatccggaggat
ctggaggcagcaccaacctgtctgacatcatcgagaaggagacaggcaagcagctggtcatccaggag
agcatcctgatgctgcccgaagaagtcgaagaagtgatcggaaacaagcctgagagcgatatcctggt
ccataccgcctacgacgagagtaccgacgaaaatgtgatgctgctgacatccgacgccccagagtata
agccctgggctctggtcatccaggattccaacggagagaacaaaatcaaaatgctgtctggcggctca
aaaagaaccgccgacggcagcgaattcgagcccaagaagaagaggaaagtctaaccggtcatcatcac
catcaccattgagtttaaacccgctgatcagcctcgactgtgccttctagttgccagccatctgttgt
ttgcccctcccccgtgccttccttgaccctggaaggtgccactcccactgtcctttcctaataaaatg
aggaaattgcatcgcattgtctgagtaggtgtcattctattctggggggtggggtggggcaggacagc
aagggggaggattgggaagacaatagcaggcatgctggggatgcggtgggctctatggcttctgaggc
ggaaagaaccagctggggctcgataccgtcgacctctagctagagcttggcgtaatcatggtcatagc
tgtttcctgtgtgaaattgttatccgctcacaattccacacaacatacgagccggaagcataaagtgt
aaagcctaggatgcctaatgagtgagctaactcacattaattgcgttgcgctcactgcccgctttcca
gtcgggaaacctgtcgtgccagctgcattaatgaatcggccaacgcgcgggaagaggcggtttgcgta
ttgggcgctcttccgcttcctcgctcactgactcgctgcgctcggtcgttcggctgcggcgagcggta
tcagctcactcaaaggcggtaatacggttatccacagaatcaggggataacgcaggaaagaacatgtg
agcaaaaggccagcaaaaggccaggaaccgtaaaaaggccgcgttgctggcgtttttccataggctcc
gcccccctgacgagcatcacaaaaatcgacgctcaagtcagaggtggcgaaacccgacaggactataa
agataccaggcgtttccccctggaagctccctcgtgcgctctcctgttccgaccctgccgcttaccgg
atacctgtccgcctttctcccttcgggaagcgtggcgctttctcatagctcacgctgtaggtatctca
gttcggtgtaggtcgttcgctccaagctgggctgtgtgcacgaaccccccgttcagcccgaccgctgc
gccttatccggtaactatcgtcttgagtccaacccggtaagacacgacttatcgccactggcagcagc
cactggtaacaggattagcagagcgaggtatgtaggcggtgctacagagttcttgaagtggtggccta
actacggctacactagaagaacagtatttggtatctgcgctctgctgaagccagttaccttcggaaaa
agagttggtagctcttgatccggcaaacaaaccaccgctggtagcggtggtttttttgtttgcaagca
gcagattacgcgcagaaaaaaaggatctcaagaagatcctttgatcttttctacggggtctgacactc
agtggaacgaaaactcacgttaagggattttggtcatgagattatcaaaaaggatcttcacctagatc
cttttaaattaaaaatgaagttttaaatcaatctaaagtatatatgagtaaacttggtctgacagtta
ccaatgcttaatcagtgaggcacctatctcagcgatctgtctatttcgttcatccatagttgcctgac
tccccgtcgtgtagataactacgatacgggagggcttaccatctggccccagtgctgcaatgataccg
cgagacccacgctcaccggctccagatttatcagcaataaaccagccagccggaagggccgagcgcag
aagtggtcctgcaactttatccgcctccatccagtctattaattgttgccgggaagctagagtaagta
gttcgccagttaatagtttgcgcaacgttgttgccattgctacaggcatcgtggtgtcacgctcgtcg
tttggtatggcttcattcagctccggttcccaacgatcaaggcgagttacatgatcccccatgttgtg
caaaaaagcggttagctccttcggtcctccgatcgttgtcagaagtaagttggccgcagtgttatcac
tcatggttatggcagcactgcataattctcttactgtcatgccatccgtaagatgcttttctgtgact
ggtgagtactcaaccaagtcattctgagaatagtgtatgcggcgaccgagttgctcttgcccggcgtc
aatacgggataataccgcgccacatagcagaactttaaaagtgctcatcattggaaaacgttcttcgg
ggcgaaaactctcaaggatcttaccgctgttgagatccagttcgatgtaacccactcgtgcacccaac
tgatcttcagcatcttttactttcaccagcgtttctgggtgagcaaaaacaggaaggcaaaatgccgc
aaaaaagggaataagggcgacacggaaatgttgaatactcatactcttcctttttcaatattattgaa
gcatttatcagggttattgtctcatgagcggatacatatttgaatgtatttagaaaaataaacaaata
ggggttccgcgcacatttccccgaaaagtgccacctgacgtcgacggatcgggagatcgatctcccga
tcccctagggtcgactctcagtacaatctgctctgatgccgcatagttaagccagtatctgctccctg
cttgtgtgttggaggtcgctgagtagtgcgcgagcaaaatttaagctacaacaaggcaaggcttgacc
gacaattgcatgaagaatctgcttagggttaggcgttttgcgctgcttcgcgatgtacgggccagata
tacgcgttgacattgattattgactagttattaatagtaatcaattacggggtcattagttcatagcc
catatatggagttccgcgttacataacttacggtaaatggcccgcctggctgaccgcccaacgacccc
cgcccattgacgtcaataatgacgtatgttcccatagtaacgccaatagggactttccattgacgtca
atgggtggagtatttacggtaaactgcccacttggcagtacatcaagtgtatc
Target sequence of the Exon 44 gRNA (SEQ ID NO: 9)
cgcctgcaggtaaaagcata
PAM (SEQ ID NO: 10)
NGG
PAM (SEQ ID NO: 11)
NNNRRT
PAM (SEQ ID NO: 12)
NNGRR (R = A or G)
PAM (SEQ ID NO: 13)
NNGRRN (R = A or G)
PAM (SEQ ID NO: 14)
NNGRRT (R = A or G)
PAM (SEQ ID NO: 15)
NNGRRV (R = A or G; V = A, C, or G)
RT-PCR primer (SEQ ID NO: 16)
CTACAACAAAGCTCAGGTCG
RT-PCR primer (SEQ ID NO: 17)
TTCTCAGGTAAAGCTCTGGAAAC
Polynucleotide encoding UGI-2 (SEQ ID NO: 18)
accaacctgtctgacatcatcgagaaggagacaggcaagcagctggtcatccaggagagcatcctgat
gctgcccgaagaagtcgaagaagtgatcggaaacaagcctgagagcgatatcctggtccataccgcct
acgacgagagtaccgacgaaaatgtgatgctgctgacatccgacgccccagagtataagccctgggct
ctggtcatccaggattccaacggagagaacaaaatcaaaatgctg
PAM (SEQ ID NO: 19)
NGA
UGI polypeptide (SEQ ID NO: 20)
TNLSDIIEKETGKQLVIQESILMLPEEVEEVIGNKPESDILVHTAYDESTDENVMLLTSDAPEYKPWA
LVIQDSNGENKIKML
ABE7.9
(Gaudelli et al. Nature 2017, 551, 464-471)
ABE7.9 (ecTadA(wt)-linker(32 aa)-ecTadA*(7.9)-linker(32 aa)-Cas9 nickase-NLS):
lowercase double underline = ecTadA (wt), monomer 1 of 2
lowercase, underlined = linker
CAPS UNDERLINED = evolved ecTadA* internal monomer 2 of 2, with mutations
highlighted in BOLD
CAPS = Cas9 nickase (D10A mutation underlined)
lowercase = NLS
Protein (SEQ ID NO: 27):
msevefsheywmrhaltlakrawderevpvgavlvhnnrvigegwnrpigrhdptahaeimalrqgglvmqnyrlidatlyvtle
pcvmcagamihsrigrvvfgardaktgaagslmdvihhpgmnhrveitegiladecaallsdffrmrrgeikaqkkaqsstdsg
gssggssgsetpgtsesatpessggssggsSEVEFSHEYWMRHALTLAKRALDEREVPVGAVLVLNNR
VIGEGWNRAIGLHDPTAHAEIMALRQGGLVMQNYRLIDATLYVTFEPCVMCAGAMIHSRIG
RVVFGVRNAKTGAAGSLMDVLHYPGMNHRVEITEGILADECNALLCYFFRMPRQVFNAQK
KAQSSTDsggssggssgsetpgtsesatpessggssggsDKKYSIGLAIGTNSVGWAVITDEYKVPSKKF
KVLGNTDRHSIKKNLIGALLFDSGETAEATRLKRTARRRYTRRKNRICYLQEIFSNEMAKVD
DSFFHRLEESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSARLSKSRR
LENLIAQLPGEKKNGLFGNLIALSLGLTPNFKSNFDLAEDAKLQLSKDTYDDDLDNLLAQIGD
QYADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSIP
HQIHLGELHAILRRQEDFYPFLKQNREKIEKILTFRIPYYVGPLARGNSRFAWMTRKSEETITP
WNFEEVVDKGASAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDRFNASLGTYHDLLK
IIKDKDFLDNEENEDILEDIVLTLTLFEDREMIEERLKTYAHLFDDKVMKQLKRRRYTGWGRL
SRKLINGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGLSE
LDKAGFIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEIGKATAK
YFFYSNIMNFFKTEITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAKVEKGKSKKLKS
VKELLGITIMERSSFEKNPIDFLEAKGYKEVKKDLIIKLPKYSLFELENGRKRMLASAGELQKG
NELALPSKYVNFLYLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRYTSTKEVLDATLIHQ
SITGLYETRIDLSQLGGDsggspkkkrkv*
DNA (SEQ ID NO: 35):
atgtccgaagtcgagttttcccatgagtactggatgagacacgcattgactctcgcaaagagggcttgggatgaacgcgaggtgc
ccgtgggggcagtactcgtgcataacaatcgcgtaatcggcgaaggttggaataggccgatcggacgccacgaccccactgc
acatgcggaaatcatggcccttcgacagggagggcttgtgatgcagaattatcgacttatcgatgcgacgctgtacgtcacgcttg
aaccttgcgtaatgtgcgcgggagctatgattcactcccgcattggacgagttgtattcggtgcccgcgacgccaagacgggtgc
cgcaggttcactgatggacgtgctgcatcacccaggcatgaaccaccgggtagaaatcacagaaggcatattggcggacgaa
tgtgcggcgctgttgtccgacttttttcgcatgcggaggcaggagatcaaggcccagaaaaaagcacaatcctctactgactctg
gtggttcttctggtggttctagcggcagcgagactcccgggacctcagagtccgccacacccgaaagttctggtggttcttctggtg
gttctTCCGAAGTCGAGTTTTCCCATGAGTACTGGATGAGACACGCATTGACTCTCGCAAA
GAGGGCTCTCGATGAACGCGAGGTGCCCGTGGGGGCAGTACTCGTGCTCAACAATCG
CGTAATCGGCGAAGGTTGGAATAGGGCAATCGGACTCCACGACCCCACTGCACATGCG
GAAATCATGGCCCTTCGACAGGGAGGGCTTGTGATGCAGAATTATCGACTTATCGATG
CGACGCTGTACGTCACGTITGAACCTTGCGTAATGTGCGCGGGACCTATGATTCACTC
CCGCATTGGACGAGTTGTATTCGGTGTTCGCAACGCCAAGACGGGTGCCGCAGGTTCA
CTGATGGACGTGCTGCATTACCCAGGCATGAACCACCGGGTAGAAATCACAGAAGGCA
TATTGGCGGACGAATGTAACGCGCTGTTGTGTTACTTTTTCGCATGCCCAGGCAGGTC
TTTAACGCCCAGAAAAAAGCACAATCCTCTACTGACtctggtggttcttctggtggttctagcggcagcgag
actcccgggacctcagagtccgccacacccgaaagttctggtggttcttctggtggttctGATAAAAAGTATTCTATTG
GTTTAGCCATCGGCACTAATTCCGTTGGATGGGCTGTCATAACCGATGAATACAAAGTA
CCTTCAAAGAAATTTAAGGTGTTGGGGAACACAGACCGTCATTCGATTAAAAAGAATCTT
ATCGGTGCCCTCCTATTCGATAGTGGCGAAACGGCAGAGGCGACTCGCCTGAAACGAA
CCGCTCGGAGAAGGTATACACGTCGCAAGAACCGAATATGTTACTTACAAGAAATTTTT
AGCAATGAGATGGCCAAAGTTGACGATTCTTTCTTTCACCGTTTGGAAGAGTCCTTCCT
TGTCGAAGAGGACAAGAAACATGAACGGCACCCCATCTTTGGAAACATAGTAGATGAG
GTGGCATATCATGAAAAGTACCCAACGATTTATCACCTCAGAAAAAAGCTAGTTGACTC
AACTGATAAAGCGGACCTGAGGTTAATCTACTTGGCTCTTGCCCATATGATAAAGTTCC
GTGGGCACTTTCTCATTGAGGGTGATCTAAATCCGGACAACTCGGATGTCGACAAACTG
TTCATCCAGTTAGTACAAACCTATAATCAGTTGTTTGAAGAGAACCCTATAAATGCAAGT
GGCGTGGATGCGAAGGCTATTCTTAGCGCCCGCCTCTCTAAATCCCGACGGCTAGAAA
ACCTGATCGCACAATTACCCGGAGAGAAGAAAAATGGGTTGTTCGGTAACCTTATAGCG
CTCTCACTAGGCCTGACACCAAATTTTAAGTCGAACTTCGACTTAGCTGAAGATGCCAA
ATTGCAGCTTAGTAAGGACACGTACGATGACGATCTCGACAATCTACTGGCACAAATTG
GAGATCAGTATGCGGACTTATTTTTGGCTGCCAAAAACCTTAGCGATGCAATCCTCCTA
TCTGACATACTGAGAGTTAATACTGAGATTACCAAGGCGCCGTTATCCGCTTCAATGAT
CAAAAGGTACGATGAACATCACCAAGACTTGACACTTCTCAAGGCCCTAGTCCGTCAGC
AACTGCCTGAGAAATATAAGGAAATATTCTTTGATCAGTCGAAAAACGGGTACGCAGGT
TATATTGACGGCGGAGCGAGTCAAGAGGAATTCTACAAGTTTATCAAACCCATATTAGA
GAAGATGGATGGGACGGAAGAGTTGCTTGTAAAACTCAATCGCGAAGATCTACTGCGA
AAGCAGCGGACTTTCGACAACGGTAGCATTCCACATCAAATCCACTTAGGCGAATTGCA
TGCTATACTTAGAAGGCAGGAGGATTTTTATCCGTTCCTCAAAGACAATCGTGAAAAGA
TTGAGAAAATCCTAACCTTTCGCATACCTTACTATGTGGGACCCCTGGCCCGAGGGAAC
TCTCGGTTCGCATGGATGACAAGAAAGTCCGAAGAAACGATTACTCCATGGAATTTTGA
GGAAGTTGTCGATAAAGGTGCGTCAGCTCAATCGTTCATCGAGAGGATGACCAACTTTG
ACAAGAATTTACCGAACGAAAAAGTATTGCCTAAGCACAGTTTACTTTACGAGTATTTCA
CAGTGTACAATGAACTCACGAAAGTTAAGTATGTCACTGAGGGCATGCGTAAACCCGCC
TTTCTAAGCGGAGAACAGAAGAAAGCAATAGTAGATCTGTTATTCAAGACCAACCGCAA
AGTGACAGTTAAGCAATTGAAAGAGGACTACTTTAAGAAAATTGAATGCTTCGATTCTGT
CGAGATCTCCGGGGTAGAAGATCGATTTAATGCGTCACTTGGTACGTATCATGACCTCC
TAAAGATAATTAAAGATAAGGACTTCCTGGATAACGAAGAGAATGAAGATATCTTAGAAG
ATATAGTGTTGACTCTTACCCTCTTTGAAGATCGGGAAATGATTGAGGAAAGACTAAAAA
CATACGCTCACCTGTTCGACGATAAGGTTATGAAACAGTTAAAGAGGCGTCGCTATACG
GGCTGGGGACGATTGTCGCGGAAACTTATCAACGGGATAAGAGACAAGCAAAGTGGTA
AAACTATTCTCGATTTTCTAAAGAGCGACGGCTTCGCCAATAGGAACTTTATGCAGCTG
ATCCATGATGACTCTTTAACCTTCAAAGAGGATATACAAAAGGCACAGGTTTCCGGACA
AGGGGACTCATTGCACGAACATATTGCGAATCTTGCTGGTTCGCCAGCCATCAAAAAG
GGCATACTCCAGACAGTCAAAGTAGTGGATGAGCTAGTTAAGGTCATGGGACGTCACA
AACCGGAAAACATTGTAATCGAGATGGCACGCGAAAATCAAACGACTCAGAAGGGGCA
AAAAAACAGTCGAGAGCGGATGAAGAGAATAGAAGAGGGTATTAAAGAACTGGGCAGC
CAGATCTTAAAGGAGCATCCTGTGGAAAATACCCAATTGCAGAACGAGAAACTTTACCT
CTATTACCTACAAAATGGAAGGGACATGTATGTTGATCAGGAACTGGACATAAACCGTT
TATCTGATTACGACGTCGATCACATTGTACCCCAATCCTTTTTGAAGGACGATTCAATCG
ACAATAAAGTGCTTACACGCTCGGATAAGAACCGAGGGAAAAGTGACAATGTTCCAAGC
GAGGAAGTCGTAAAGAAAATGAAGAACTATTGGCGGCAGCTCCTAAATGCGAAACTGAT
AACGCAAAGAAAGTTCGATAACTTAACTAAAGCTGAGAGGGGTGGCTTGTCTGAACTTG
ACAAGGCCGGATTTATTAAACGTCAGCTCGTGGAAACCCGCCAAATCACAAAGCATGTT
GCACAGATACTAGATTCCCGAATGAATACGAAATACGACGAGAACGATAAGCTGATTCG
GGAAGTCAAAGTAATCACTTTAAAGTCAAAATTGGTGTCGGACTTCAGAAAGGATTTTCA
ATTCTATAAAGTTAGGGAGATAAATAACTACCACCATGCGCACGACGCTTATCTTAATGC
CGTCGTAGGGACCGCACTCATTAAGAAATACCCGAAGCTAGAAAGTGAGTTTGTGTATG
GTGATTACAAAGTTTATGACGTCCGTAAGATGATCGCGAAAAGCGAACAGGAGATAGG
CAAGGCTACAGCCAAATACTTCTTTTATTCTAACATTATGAATTTCTTTAAGACGGAAATC
ACTCTGGCAAACGGAGAGATACGCAAACGACCTTTAATTGAAACCAATGGGGAGACAG
GTGAAATCGTATGGGATAAGGGCCGGGACTTCGCGACGGTGAGAAAAGTTTTGTCCAT
GCCCCAAGTCAACATAGTAAAGAAAACTGAGGTGCAGACCGGAGGGTTTTCAAAGGAA
TCGATTCTTCCAAAAAGGAATAGTGATAAGCTCATCGCTCGTAAAAAGGACTGGGACCC
GAAAAAGTACGGTGGCTTCGATAGCCCTACAGTTGCCTATTCTGTCCTAGTAGTGGCAA
AAGTTGAGAAGGGAAAATCCAAGAAACTGAAGTCAGTCAAAGAATTATTGGGGATAACG
ATTATGGAGCGCTCGTCTTTTGAAAAGAACCCCATCGACTTCCTTGAGGCGAAAGGTTA
CAAGGAAGTAAAAAAGGATCTCATAATTAAACTACCAAAGTATAGTCTGTTTGAGTTAGA
AAATGGCCGAAAACGGATGTTGGCTAGCGCCGGAGAGCTTCAAAAGGGGAACGAACTC
GCACTACCGTCTAAATACGTGAATTTCCTGTATTTAGCGTCCCATTACGAGAAGTTGAAA
GGTTCACCTGAAGATAACGAACAGAAGCAACTTTTTGTTGAGCAGCACAAACATTATCT
CGACGAAATCATAGAGCAAATTTCGGAATTCAGTAAGAGAGTCATCCTAGCTGATGCCA
ATCTGGACAAAGTATTAAGCGCATACAACAAGCACAGGGATAAACCCATACGTGAGCAG
GCGGAAAATATTATCCATTTGTTTACTCTTACCAACCTCGGCGCTCCAGCCGCATTCAA
GTATTTTGACACAACGATAGATCGCAAACGATACACTTCTACCAAGGAGGTGCTAGACG
CGACACTGATTCACCAATCCATCACGGGATTATATGAAACTCGGATAGATTTGTCACAG
CTTGGGGGTGACtctggtggttctcccaagaagaagaggaaagtcTAA
ABE7.10
(Gaudelli et al. Nature 2017, 551, 464-471)
ABE7.10 (ecTadA(wt)-linker(32 aa)-ecTadA*(7.10)-linker(32 aa)-Cas9 nickase-NLS):
lowercase double underline = ecTadA (wt), monomer 1 of 2
lowercase, underlined = linker
CAPS UNDERLINED = evolved ecTadA* internal monomer 2 of 2, with mutations
highlighted in BOLD
CAPS = Cas9 nickase (D10A mutation underlined)
lowercase = NLS
Protein (SEQ ID NO: 28):
msevefsheywmrhaltlakrawderevpvgavivhnnrvigegwnrpigrhdptahaeimalrqgglvmgnyrlidatiyvtle
pcvmcagamihsrigryyfgardaktgaagslmdvihhpgmnhrveitegiladecaallsdffrmrrgeikaqkkagsstdsg
gssggssgsetpgtsesatpessggssggsSEVEFSHEYWMRHALTLAKRARDEREVPVGAVLVLNN
RVIGEGWNRAIGLHDPTAHAEIMALRQGGLVMQNYRLIDATLYVTFEPCVMCAGAMIHSRI
GRVVFGVRNAKTGAAGSLMDVLHYPGMNHRVEITEGILADECAALLCYFERMPRQVFNAQ
KKAQSSTDsggssggssgsetpgtsesatpessggssggsDKKYSIGLAIGTNSVGWAVITDEYKVPSK
KFKVLGNTDRHSIKKNLIGALLFDSGETAEATRLKRTARRRYTRRKNRICYLQEIFSNEMAKV
DDSFFHRLEESFLVEEDKKHERHPIFGNIVDEVAYHEKYPTIYHLRKKLVDSTDKADLRLIYL
ALAHMIKFRGHFLIEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKDTYDDDLDNLLAQI
GDQYADLFLAAKNLSDAILLSDILRVNTEITKAPLSASMIKRYDEHHQDLTLLKALVRQQLPEK
YKEIFFDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTRKSEE
TITPWNFEEVVDKGASAQSFIERMTNFDKNLPNEKVLPKHSLLYEYFTVYNELTKVKYVTEG
MRKPAFLSGEQKKAIVDLLFKTNRKVTVKQLKEDYFKKIECFDSVEISGVEDRFNASLGTYH
DLLKIIKDKDFLDNEENEDILEDIVLTLTLFEDREMIEERLKTYAHLFDDKVMKQLKRRRYTGW
GRLSRKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSRERMKR
IEEGIKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAERGG
LSELDKAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSEQEIGKA
TAKYFFYSNIMNFFKTEITLANGEIRKRPLIETNGETGEIVWDKGRDFATVRKVLSMPQVNIV
KKTEVQTGGFSKESILPKRNSDKLIARKKDWDPKKYGGFDSPTVAYSVLVVAKVEKGKSKK
LKSVKELLGITIMERSSFEKNPIDFLEAKGYKEVKKDLIIKLPKYSLFELENGRKRMLASAGEL
QKGNELALPSKYVNFLYLASHYEKLKGSPEDNEQKQLFVEQHKHYLDEIIEQISEFSKRVILA
DANLDKVLSAYNKHRDKPIREQAENIIHLFTLTNLGAPAAFKYFDTTIDRKRYTSTKEVLDATL
IHQSITGLYETRIDLSQLGGDsggspkkkrkv*
DNA (SEQ ID NO: 36):
atgtccgaagtcgagttttcccatgagtactggatgagacacgcattgactctcgcaaagagggcttoggatgaacacgagatgc
ccgtgggggcagtactcgtgcataacaatcgcgtaatcggcgaaggttggaataggccgatcggacgccacgaccccactgc
acatgcggaaatcatggcccttcgacagggagggcttgtgatgcagaattatcgacttatcgatgcgacgctgtacgtcacgcttg
aaccttgcgtaatgtgcgcgggagctatgattcactcccgcattggacgagttgtattcggtgcccgcgacgccaagacgggtgc
cgcaggttcactgatggacgtgctgcatcacccaggcatgaaccaccgggtagaaatcacagaaggcatattggcggacgaa
tgtgcggcgctgttgtccgacttttttcgcatgcggaggcaggagatcaaggcccagaaaaaagcacaatcctctactgactctg
gtggttcttctggtggttctagcggcagcgagactcccgggacctcagagtccgccacacccgaaagttctggtggttcttctggtg
gttctTCCGAAGTCGAGTTTTCCCATGAGTACTGGATGAGACACGCATTGACTCTCGCAAA
GAGGGCTCGAGATGAACGCGAGGTGCCCGTGGGGGCAGTACTCGTGCTCAACAATCG
CGTAATCGGCGAAGGTTGGAATAGGGCAATCGGACTCCACGACCCCACTGCACATGCG
GAAATCATGGCCCTTCGACAGGGAGGGCTTGTGATGCAGAATTATCGACTTATCGATG
CGACGCTGTACGTCACGTTTGAACCTTGCGTAATGTGCGCGGGAGCTATGATTCACTC
CCGCATTGGACGAGTTGTATTCGGTGTTCGCAACGCCAAGACGGGTGCCGCAGGTTCA
CTGATGGACGTGCTGCATTACCCAGGCATGAACCACCGGGTAGAAATCACAGAAGGCA
TATTGGCGGACGAATGTGCGGCGCTGTTGTGTTACTTTTTTCGCATGCCCAGGCAGGT
CTTTAACGCCCAGAAAAAAGCACAATCCTCTACTGACtctggtggttcttctggtggttctagcggcagcg
agactcccgggacctcagagtccgccacacccgaaagttctggtggttcttctggtggttctGATAAAAAGTATTCTATT
GGTTTAGCCATCGGCACTAATTCCGTTGGATGGGCTGTCATAACCGATGAATACAAAGT
ACCTTCAAAGAAATTTAAGGTGTTGGGGAACACAGACCGTCATTCGATTAAAAAGAATC
TTATCGGTGCCCTCCTATTCGATAGTGGCGAAACGGCAGAGGCGACTCGCCTGAAACG
AACCGCTCGGAGAAGGTATACACGTCGCAAGAACCGAATATGTTACTTACAAGAAATTT
TTAGCAATGAGATGGCCAAAGTTGACGATTCTTTCTTTCACCGTTTGGAAGAGTCCTTC
CTTGTCGAAGAGGACAAGAAACATGAACGGCACCCCATCTTTGGAAACATAGTAGATGA
GGTGGCATATCATGAAAAGTACCCAACGATTTATCACCTCAGAAAAAAGCTAGTTGACT
CAACTGATAAAGCGGACCTGAGGTTAATCTACTTGGCTCTTGCCCATATGATAAAGTTC
CGTGGGCACTTTCTCATTGAGGGTGATCTAAATCCGGACAACTCGGATGTCGACAAACT
GTTCATCCAGTTAGTACAAACCTATAATCAGTTGTTTGAAGAGAACCCTATAAATGCAAG
TGGCGTGGATGCGAAGGCTATTCTTAGCGCCCGCCTCTCTAAATCCCGACGGCTAGAA
AACCTGATCGCACAATTACCCGGAGAGAAGAAAAATGGGTTGTTCGGTAACCTTATAGC
GCTCTCACTAGGCCTGACACCAAATTTTAAGTCGAACTTCGACTTAGCTGAAGATGCCA
AATTGCAGCTTAGTAAGGACACGTACGATGACGATCTCGACAATCTACTGGCACAAATT
GGAGATCAGTATGCGGACTTATTTTTGGCTGCCAAAAACCTTAGCGATGCAATCCTCCT
ATCTGACATACTGAGAGTTAATACTGAGATTACCAAGGCGCCGTTATCCGCTTCAATGA
TCAAAAGGTACGATGAACATCACCAAGACTTGACACTTCTCAAGGCCCTAGTCCGTCAG
CAACTGCCTGAGAAATATAAGGAAATATTCTTTGATCAGTCGAAAAACGGGTACGCAGG
TTATATTGACGGCGGAGCGAGTCAAGAGGAATTCTACAAGTTTATCAAACCCATATTAG
AGAAGATGGATGGGACGGAAGAGTTGCTTGTAAAACTCAATCGCGAAGATCTACTGCG
AAAGCAGCGGACTTTCGACAACGGTAGCATTCCACATCAAATCCACTTAGGCGAATTGC
ATGCTATACTTAGAAGGCAGGAGGATTTTTATCCGTTCCTCAAAGACAATCGTGAAAAG
ATTGAGAAAATCCTAACCTTTCGCATACCTTACTATGTGGGACCCCTGGCCCGAGGGAA
CTCTCGGTTCGCATGGATGACAAGAAAGTCCGAAGAAACGATTACTCCATGGAATTTTG
AGGAAGTTGTCGATAAAGGTGCGTCAGCTCAATCGTTCATCGAGAGGATGACCAACTTT
GACAAGAATTTACCGAACGAAAAAGTATTGCCTAAGCACAGTTTACTTTACGAGTATTTC
ACAGTGTACAATGAACTCACGAAAGTTAAGTATGTCACTGAGGGCATGCGTAAACCCGC
CTTTCTAAGCGGAGAACAGAAGAAAGCAATAGTAGATCTGTTATTCAAGACCAACCGCA
AAGTGACAGTTAAGCAATTGAAAGAGGACTACTTTAAGAAAATTGAATGCTTCGATTCTG
TCGAGATCTCCGGGGTAGAAGATCGATTTAATGCGTCACTTGGTACGTATCATGACCTC
CTAAAGATAATTAAAGATAAGGACTTCCTGGATAACGAAGAGAATGAAGATATCTTAGAA
GATATAGTGTTGACTCTTACCCTCTTTGAAGATCGGGAAATGATTGAGGAAAGACTAAA
AACATACGCTCACCTGTTCGACGATAAGGTTATGAAACAGTTAAAGAGGCGTCGCTATA
CGGGCTGGGGACGATTGTCGCGGAAACTTATCAACGGGATAAGAGACAAGCAAAGTG
GTAAAACTATTCTCGATTTTCTAAAGAGCGACGGCTTCGCCAATAGGAACTTTATGCAG
CTGATCCATGATGACTCTTTAACCTTCAAAGAGGATATACAAAAGGCACAGGTTTCCGG
ACAAGGGGACTCATTGCACGAACATATTGCGAATCTTGCTGGTTCGCCAGCCATCAAAA
AGGGCATACTCCAGACAGTCAAAGTAGTGGATGAGCTAGTTAAGGTCATGGGACGTCA
CAAACCGGAAAACATTGTAATCGAGATGGCACGCGAAAATCAAACGACTCAGAAGGGG
CAAAAAAACAGTCGAGAGCGGATGAAGAGAATAGAAGAGGGTATTAAAGAACTGGGCA
GCCAGATCTTAAAGGAGCATCCTGTGGAAAATACCCAATTGCAGAACGAGAAACTTTAC
CTCTATTACCTACAAAATGGAAGGGACATGTATGTTGATCAGGAACTGGACATAAACCG
TTTATCTGATTACGACGTCGATCACATTGTACCCCAATCCTTTTTGAAGGACGATTCAAT
CGACAATAAAGTGCTTACACGCTCGGATAAGAACCGAGGGAAAAGTGACAATGTTCCAA
GCGAGGAAGTCGTAAAGAAAATGAAGAACTATTGGCGGCAGCTCCTAAATGCGAAACT
GATAACGCAAAGAAAGTTCGATAACTTAACTAAAGCTGAGAGGGGTGGCTTGTCTGAAC
TTGACAAGGCCGGATTTATTAAACGTCAGCTCGTGGAAACCCGCCAAATCACAAAGCAT
GTTGCACAGATACTAGATTCCCGAATGAATACGAAATACGACGAGAACGATAAGCTGAT
TCGGGAAGTCAAAGTAATCACTTTAAAGTCAAAATTGGTGTCGGACTTCAGAAAGGATT
TTCAATTCTATAAAGTTAGGGAGATAAATAACTACCACCATGCGCACGACGCTTATCTTA
ATGCCGTCGTAGGGACCGCACTCATTAAGAAATACCCGAAGCTAGAAAGTGAGTTTGT
GTATGGTGATTACAAAGTTTATGACGTCCGTAAGATGATCGCGAAAAGCGAACAGGAGA
TAGGCAAGGCTACAGCCAAATACTTCTTTTATTCTAACATTATGAATTTCTTTAAGACGG
AAATCACTCTGGCAAACGGAGAGATACGCAAACGACCTTTAATTGAAACCAATGGGGAG
ACAGGTGAAATCGTATGGGATAAGGGCCGGGACTTCGCGACGGTGAGAAAAGTTTTGT
CCATGCCCCAAGTCAACATAGTAAAGAAAACTGAGGTGCAGACCGGAGGGTTTTCAAA
GGAATCGATTCTTCCAAAAAGGAATAGTGATAAGCTCATCGCTCGTAAAAAGGACTGGG
ACCCGAAAAAGTACGGTGGCTTCGATAGCCCTACAGTTGCCTATTCTGTCCTAGTAGTG
GCAAAAGTTGAGAAGGGAAAATCCAAGAAACTGAAGTCAGTCAAAGAATTATTGGGGAT
AACGATTATGGAGCGCTCGTCTTTTGAAAAGAACCCCATCGACTTCCTTGAGGCGAAAG
GTTACAAGGAAGTAAAAAAGGATCTCATAATTAAACTACCAAAGTATAGTCTGTTTGAGT
TAGAAAATGGCCGAAAACGGATGTTGGCTAGCGCCGGAGAGCTTCAAAAGGGGAACGA
ACTCGCACTACCGTCTAAATACGTGAATTTCCTGTATTTAGCGTCCCATTACGAGAAGTT
GAAAGGTTCACCTGAAGATAACGAACAGAAGCAACTTTTTGTTGAGCAGCACAAACATT
ATCTCGACGAAATCATAGAGCAAATTTCGGAATTCAGTAAGAGAGTCATCCTAGCTGAT
GCCAATCTGGACAAAGTATTAAGCGCATACAACAAGCACAGGGATAAACCCATACGTGA
GCAGGCGGAAAATATTATCCATTTGTTTACTCTTACCAACCTCGGCGCTCCAGCCGCAT
TCAAGTATTTTGACACAACGATAGATCGCAAACGATACACTTCTACCAAGGAGGTGCTA
GACGCGACACTGATTCACCAATCCATCACGGGATTATATGAAACTCGGATAGATTTGTC
ACAGCTTGGGGGTGACtctggtggttctcccaagaagaagaggaaagtcTAA
ABEmax
(Koblan et al. Nature Biotech. 2018, 36, 843-846)
ABEmax (NLS-ecTadA(wt)-linker(32 aa)-ecTadA*(7.10)-linker(32 aa)-Cas9 nickase-
linker-NLS):
lowercase double underline = ecTadA (wt), monomer 1 of 2
lowercase, underlined = linker
CAPS UNDERLINED = evolved ecTadA* internal monomer 2 of 2
CAPS = Cas9 nickase (D10A mutation underlined)
lowercase = NLS
Protein (SEQ ID NO: 29):
mkrtadgsefespkkkrkvsevefsheywmrhaltlakrawderevpvgavlvhnnrvigegwnrpigrhdptahaeimalrq
gglvmqnyrlidatlyvtlepcvmcagamihsrigrvvfgardaktgaagslmdvlhhpgmnhrveitegiladecaallsdffrmr
rqeikaqkkaqsstdsggssggssgsetpgtsesatpessggssggsSEVEFSHEYWMRHALTLAKRARDER
EVPVGAVLVLNNRVIGEGWNRAIGLHDPTAHAEIMALRQGGLVMQNYRLIDATLYVTFEPCV
MCAGAMIHSRIGRVVFGVRNAKTGAAGSLMDVLHYPGMNHRVEITEGILADECAALLCYFF
RMPRQVFNAQKKAQSSTDsggssggssgsetpgtsesatpessggssggsDKKYSIGLAIGTNSVGWA
VITDEYKVPSKKFKVLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTY
DDDLDNLLAQIGDQYADLFLAAKNLSDAILLSDILRVNTEITKAPLSASMIKRYDEHHQDLTLL
KALVRQQLPEKYKEIFFDQSKNGYAGYIDGGASQEEFYKFIKPILEKMDGTEELLVKLNREDL
LRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIECFDSVEISGVED
RFNASLGTYHDLLKIIKDKDFLDNEENEDILEDIVLTLTLFEDREMIEERLKTYAHLFDDKVMK
QLKRRRYTGWGRLSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVIEMARENQTTQKG
QKNSRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLK
SKLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFELENGR
KRMLASAGELQKGNELALPSKYVNFLYLASHYEKLKGSPEDNEQKQLFVEQHKHYLDEIIEQ
ISEFSKRVILADANLDKVLSAYNKHRDKPIREQAENIIHLFTLTNLGAPAAFKYFDTTIDRKRYT
STKEVLDATLIHQSITGLYETRIDLSQLGGDsggskrtadgsefepkkkrkv*
DNA (SEQ ID NO: 37):
atgaaacggacagccgacggaagcgagttcgagtcaccaaagaagaagcggaaagtctctgaagtcgagtttagccacga
gtattggatgaggcacgcactgaccctggcaaagcgagcatgggatgaaagagaagtccccgtgggcgccgtgctggtgcac
aacaatagagtgatcggagagggatggaacaggccaatcggccgccacgaccctaccgcacacgcagagatcatggcact
gaggcagggaggcctggtcatgcagaattaccgcctgatcgatgccaccctgtatgtgacactggagccatgcgtgatgtgcgc
aggagcaatgatccacagcaggatcggaagagtggtgttcggagcacgggacgccaagaccogcgcagcaggctccctga
tggatgtgctgcaccaccccggcatgaaccaccgggtggagatcacagagggaatcctggcagacgagtgcgccgccctgct
gagcgatttctttagaatgcggagacaggagatcaaggcccagaagaaggcacagagctccaccgactctggaggatctagc
ggaggatcctctggaagcgagacaccaggcacaagcgagtccgccacaccagagagctccggcggctcctccggaggatc
cTCTGAGGTGGAGTTTTCCCACGAGTACTGGATGAGACATGCCCTGACCCTGGCCAAG
AGGGCACGCGATGAGAGGGAGGTGCCTGTGGGAGCCGTGCTGGTGCTGAACAATAGA
GTGATCGGCGAGGGCTGGAACAGAGCCATCGGCCTGCACGACCCAACAGCCCATGCC
GAAATTATGGCCCTGAGACAGGGCGGCCTGGTCATGCAGAACTACAGACTGATTGACG
CCACCCTGTACGTGACATTCGAGCCTTGCGTGATGTGCGCCGGCGCCATGATCCACTC
TAGGATCGGCCGCGTGGTGTTTGGCGTGAGGAACGCAAAAACCGGCGCCGCAGGCTC
CCTGATGGACGTGCTGCACTACCCCGGCATGAATCACCGCGTCGAAATTACCGAGGGA
ATCCTGGCAGATGAATGTGCCGCCCTGCTGTGCTATTTCTTTCGGATGCCTAGACAGGT
GTTCAATGCTCAGAAGAAGGCCCAGAGCTCCACCGACtccggaggatctagcggaggctcctctggct
ctgagacacctggcacaagcgagagcgcaacacctgaaagcagcgggggcagcagcggggggtcaGACAAGAAG
TACAGCATCGGCCTGGCCATCGGCACCAACTCTGTGGGCTGGGCCGTGATCACCGAC
GAGTACAAGGTGCCCAGCAAGAAATTCAAGGTGCTGGGCAACACCGACCGGCACAGC
ATCAAGAAGAACCTGATCGGAGCCCTGCTGTTCGACAGCGGCGAAACAGCCGAGGCC
ACCCGGCTGAAGAGAACCGCCAGAAGAAGATACACCAGACGGAAGAACCGGATCTGC
TATCTGCAAGAGATCTTCAGCAACGAGATGGCCAAGGTGGACGACAGCTTCTTCCACA
GACTGGAAGAGTCCTTCCTGGTGGAAGAGGATAAGAAGCACGAGCGGCACCCCATCTT
CGGCAACATCGTGGACGAGGTGGCCTACCACGAGAAGTACCCCACCATCTACCACCTG
AGAAAGAAACTGGTGGACAGCACCGACAAGGCCGACCTGCGGCTGATCTATCTGGCC
CTGGCCCACATGATCAAGTTCCGGGGCCACTTCCTGATCGAGGGCGACCTGAACCCC
GACAACAGCGACGTGGACAAGCTGTTCATCCAGCTGGTGCAGACCTACAACCAGCTGT
TCGAGGAAAACCCCATCAACGCCAGCGGCGTGGACGCCAAGGCCATCCTGTCTGCCA
GACTGAGCAAGAGCAGACGGCTGGAAAATCTGATCGCCCAGCTGCCCGGCGAGAAGA
AGAATGGCCTGTTCGGAAACCTGATTGCCCTGAGCCTGGGCCTGACCCCCAACTTCAA
GAGCAACTTCGACCTGGCCGAGGATGCCAAACTGCAGCTGAGCAAGGACACCTACGA
CGACGACCTGGACAACCTGCTGGCCCAGATCGGCGACCAGTACGCCGACCTGTTTCT
GGCCGCCAAGAACCTGTCCGACGCCATCCTGCTGAGCGACATCCTGAGAGTGAACAC
CGAGATCACCAAGGCCCCCCTGAGCGCCTCTATGATCAAGAGATACGACGAGCACCAC
CAGGACCTGACCCTGCTGAAAGCTCTCGTGCGGCAGCAGCTGCCTGAGAAGTACAAAG
AGATTTTCTTCGACCAGAGCAAGAACGGCTACGCCGGCTACATTGACGGCGGAGCCAG
CCAGGAAGAGTTCTACAAGTTCATCAAGCCCATCCTGGAAAAGATGGACGGCACCGAG
GAACTGCTCGTGAAGCTGAACAGAGAGGACCTGCTGCGGAAGCAGCGGACCTTCGAC
AACGGCAGCATCCCCCACCAGATCCACCTGGGAGAGCTGCACGCCATTCTGCGGCGG
CAGGAAGATTTTTACCCATTCCTGAAGGACAACCGGGAAAAGATCGAGAAGATCCTGAC
CTTCCGCATCCCCTACTACGTGGGCCCTCTGGCCAGGGGAAACAGCAGATTCGCCTGG
ATGACCAGAAAGAGCGAGGAAACCATCACCCCCTGGAACTTCGAGGAAGTGGTGGACA
AGGGCGCTTCCGCCCAGAGCTTCATCGAGCGGATGACCAACTTCGATAAGAACCTGCC
CAACGAGAAGGTGCTGCCCAAGCACAGCCTGCTGTACGAGTACTTCACCGTGTATAAC
GAGCTGACCAAAGTGAAATACGTGACCGAGGGAATGAGAAAGCCCGCCTTCCTGAGCG
GCGAGCAGAAAAAGGCCATCGTGGACCTGCTGTTCAAGACCAACCGGAAAGTGACCGT
GAAGCAGCTGAAAGAGGACTACTTCAAGAAAATCGAGTGCTTCGACTCCGTGGAAATCT
CCGGCGTGGAAGATCGGTTCAACGCCTCCCTGGGCACATACCACGATCTGCTGAAAAT
TATCAAGGACAAGGACTTCCTGGACAATGAGGAAAACGAGGACATTCTGGAAGATATC
GTGCTGACCCTGACACTGTTTGAGGACAGAGAGATGATCGAGGAACGGCTGAAAACCT
ATGCCCACCTGTTCGACGACAAAGTGATGAAGCAGCTGAAGCGGCGGAGATACACCG
GCTGGGGCAGGCTGAGCCGGAAGCTGATCAACGGCATCCGGGACAAGCAGTCCGGCA
AGACAATCCTGGATTTCCTGAAGTCCGACGGCTTCGCCAACAGAAACTTCATGCAGCTG
ATCCACGACGACAGCCTGACCTTTAAAGAGGACATCCAGAAAGCCCAGGTGTCCGGCC
AGGGCGATAGCCTGCACGAGCACATTGCCAATCTGGCCGGCAGCCCCGCCATTAAGA
AGGGCATCCTGCAGACAGTGAAGGTGGTGGACGAGCTCGTGAAAGTGATGGGCCGGC
ACAAGCCCGAGAACATCGTGATCGAAATGGCCAGAGAGAACCAGACCACCCAGAAGG
GACAGAAGAACAGCCGCGAGAGAATGAAGCGGATCGAAGAGGGCATCAAAGAGCTGG
GCAGCCAGATCCTGAAAGAACACCCCGTGGAAAACACCCAGCTGCAGAACGAGAAGCT
GTACCTGTACTACCTGCAGAATGGGCGGGATATGTACGTGGACCAGGAACTGGACATC
AACCGGCTGTCCGACTACGATGTGGACCATATCGTGCCTCAGAGCTTTCTGAAGGACG
ACTCCATCGACAACAAGGTGCTGACCAGAAGCGACAAGAACCGGGGCAAGAGCGACA
ACGTGCCCTCCGAAGAGGTCGTGAAGAAGATGAAGAACTACTGGCGGCAGCTGCTGAA
CGCCAAGCTGATTACCCAGAGAAAGTTCGACAATCTGACCAAGGCCGAGAGAGGCGG
CCTGAGCGAACTGGATAAGGCCGGCTTCATCAAGAGACAGCTGGTGGAAACCCGGCA
GATCACAAAGCACGTGGCACAGATCCTGGACTCCCGGATGAACACTAAGTACGACGAG
AATGACAAGCTGATCCGGGAAGTGAAAGTGATCACCCTGAAGTCCAAGCTGGTGTCCG
ATTTCCGGAAGGATTTCCAGTTTTACAAAGTGCGCGAGATCAACAACTACCACCACGCC
CACGACGCCTACCTGAACGCCGTCGTGGGAACCGCCCTGATCAAAAAGTACCCTAAGC
TGGAAAGCGAGTTCGTGTACGGCGACTACAAGGTGTACGACGTGCGGAAGATGATCGC
CAAGAGCGAGCAGGAAATCGGCAAGGCTACCGCCAAGTACTTCTTCTACAGCAACATC
ATGAACTTTTTCAAGACCGAGATTACCCTGGCCAACGGCGAGATCCGGAAGCGGCCTC
TGATCGAGACAAACGGCGAAACCGGGGAGATCGTGTGGGATAAGGGCCGGGATTTTG
CCACCGTGCGGAAAGTGCTGAGCATGCCCCAAGTGAATATCGTGAAAAAGACCGAGGT
GCAGACAGGCGGCTTCAGCAAAGAGTCTATCCTGCCCAAGAGGAACAGCGATAAGCTG
ATCGCCAGAAAGAAGGACTGGGACCCTAAGAAGTACGGCGGCTTCGACAGCCCCACC
GTGGCCTATTCTGTGCTGGTGGTGGCCAAAGTGGAAAAGGGCAAGTCCAAGAAACTGA
AGAGTGTGAAAGAGCTGCTGGGGATCACCATCATGGAAAGAAGCAGCTTCGAGAAGAA
TCCCATCGACTTTCTGGAAGCCAAGGGCTACAAAGAAGTGAAAAAGGACCTGATCATCA
AGCTGCCTAAGTACTCCCTGTTCGAGCTGGAAAACGGCCGGAAGAGAATGCTGGCCTC
TGCCGGCGAACTGCAGAAGGGAAACGAACTGGCCCTGCCCTCCAAATATGTGAACTTC
CTGTACCTGGCCAGCCACTATGAGAAGCTGAAGGGCTCCCCCGAGGATAATGAGCAGA
AACAGCTGTTTGTGGAACAGCACAAGCACTACCTGGACGAGATCATCGAGCAGATCAG
CGAGTTCTCCAAGAGAGTGATCCTGGCCGACGCTAATCTGGACAAAGTGCTGTCCGCC
TACAACAAGCACCGGGATAAGCCCATCAGAGAGCAGGCCGAGAATATCATCCACCTGT
TTACCCTGACCAATCTGGGAGCCCCTGCCGCCTTCAAGTACTTTGACACCACCATCGAC
CGGAAGAGGTACACCAGCACCAAAGAGGTGCTGGACGCCACCCTGATCCACCAGAGC
ATCACCGGCCTGTACGAGACACGGATCGACCTGTCTCAGCTGGGAGGTGACtctggcggct
caaaaagaaccgccgacggcagcgaattcgagcccaagaagaagaggaaagtcTAA
ABE8e
(Richter et al. Nature Biotech. 2020, 38, 883-891)
ABE8e (NLS-ecTadA*(8e)-linker(32 aa)-Cas9 nickase-linker-NLS):
lowercase, underlined = linker
CAPS UNDERLINED = evolved ecTadA*
CAPS = Cas9 nickase (D10A mutation underlined)
lowercase = NLS
Protein (SEQ ID NO: 30):
mkrtadgsefespkkkrkvSEVEFSHEYWMRHALTLAKRARDEREVPVGAVLVLNNRVIGEGWNR
AIGLHDPTAHAEIMALRQGGLVMQNYRLIDATLYVTFEPCVMCAGAMIHSRIGRVVFGVRNS
KRGAAGSLMNVLNYPGMNHRVEITEGILADECAALLCDFYRMPRQVFNAQKKAQSSINsggs
sggssgsetpgtsesatpessggssggsDKKYSIGLAIGTNSVGWAVITDEYKVPSKKFKVLGNTDRHS
IKKNLIGALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQK
KAIVDLLFKTNRKVTVKOLKEDYFKKIECFDSVEISGVEDRFNASLGTYHDLLKIIKDKDFLDN
EENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIK
KGILQTVKVVDELVKVMGRHKPENIVIEMARENQTTQKGQKNSRERMKRIEEGIKELGSQIL
KEHPVENTQLQNEKLYLYYLQNGRDMYVDQELDINRLSDYDVDHIVPQSFLKDDSIDNKVLT
RSDKNRGKSDNVPSEEVVKKMKNYWRQLLNAKLITORKFDNLTKAERGGLSELDKAGFIKR
QLVETRQITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFFYSNIMN
FFKTEITLANGEIRKRPLIETNGETGEIVWDKGRDFATVRKVLSMPQVNIVKKTEVQTGGFSK
ESILPKRNSDKLIARKKDWDPKKYGGFDSPTVAYSVLVVAKVEKGKSKKLKSVKELLGITIME
RSSFEKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSAYNK
HRDKPIREQAENIIHLFTLTNLGAPAAFKYFDTTIDRKRYTSTKEVLDATLIHQSITGLYETRID
LSQLGGDsggskrtadgsefepkkkrkv*
DNA (SEQ ID NO: 38):
atgaaacggacagccgacggaagcgagttcgagtcaccaaagaagaagcggaaagtcTCTGAGGTGGAGTTTT
CCCACGAGTACTGGATGAGACATGCCCTGACCCTGGCCAAGAGGGCACGGGATGAGA
GGGAGGTGCCTGTGGGAGCCGTGCTGGTGCTGAACAATAGAGTGATCGGCGAGGGCT
GGAACAGAGCCATCGGCCTGCACGACCCAACAGCCCATGCCGAAATTATGGCCCTGA
GACAGGGCGGCCTGGTCATGCAGAACTACAGACTGATTGACGCCACCCTGTACGTGAC
ATTCGAGCCTTGCGTGATGTGCGCCGGCGCCATGATCCACTCTAGGATCGGCCGCGT
GGTGTTTGGCGTGAGGAACTCAAAAAGAGGCGCCGCAGGCTCCCTGATGAACGTGCT
GAACTACCCCGGCATGAATCACCGCGTCGAAATTACCGAGGGAATCCTGGCAGATGAA
TGTGCCGCCCTGCTGTGCGATTTCTATCGGATGCCTAGACAGGTGTTCAATGCTCAGAA
GAAGGCCCAGAGCTCCATCAACtccggaggatctagcggaggctcctctggctctgagacacctggcacaagc
gagagcgcaacacctgaaagcagcgggggcagcagcggggggtcaGACAAGAAGTACAGCATCGGCCTG
GCCATCGGCACCAACTCTGTGGGCTGGGCCGTGATCACCGACGAGTACAAGGTGCCC
AGCAAGAAATTCAAGGTGCTGGGCAACACCGACCGGCACAGCATCAAGAAGAACCTGA
TCGGAGCCCTGCTGTTCGACAGCGGCGAAACAGCCGAGGCCACCCGGCTGAAGAGAA
CCGCCAGAAGAAGATACACCAGACGGAAGAACCGGATCTGCTATCTGCAAGAGATCTT
CAGCAACGAGATGGCCAAGGTGGACGACAGCTTCTTCCACAGACTGGAAGAGTCCTTC
CTGGTGGAAGAGGATAAGAAGCACGAGCGGCACCCCATCTTCGGCAACATCGTGGAC
GAGGTGGCCTACCACGAGAAGTACCCCACCATCTACCACCTGAGAAAGAAACTGGTGG
ACAGCACCGACAAGGCCGACCTGCGGCTGATCTATCTGGCCCTGGCCCACATGATCAA
GTTCCGGGGCCACTTCCTGATCGAGGGCGACCTGAACCCCGACAACAGCGACGTGGA
CAAGCTGTTCATCCAGCTGGTGCAGACCTACAACCAGCTGTTCGAGGAAAACCCCATC
AACGCCAGCGGCGTGGACGCCAAGGCCATCCTGTCTGCCAGACTGAGCAAGAGCAGA
CGGCTGGAAAATCTGATCGCCCAGCTGCCCGGCGAGAAGAAGAATGGCCTGTTCGGA
AACCTGATTGCCCTGAGCCTGGGCCTGACCCCCAACTTCAAGAGCAACTTCGACCTGG
CCGAGGATGCCAAACTGCAGCTGAGCAAGGACACCTACGACGACGACCTGGACAACC
TGCTGGCCCAGATCGGCGACCAGTACGCCGACCTGTTTCTGGCCGCCAAGAACCTGTC
CGACGCCATCCTGCTGAGCGACATCCTGAGAGTGAACACCGAGATCACCAAGGCCCC
CCTGAGCGCCTCTATGATCAAGAGATACGACGAGCACCACCAGGACCTGACCCTGCTG
AAAGCTCTCGTGCGGCAGCAGCTGCCTGAGAAGTACAAAGAGATTTTCTTCGACCAGA
GCAAGAACGGCTACGCCGGCTACATTGACGGCGGAGCCAGCCAGGAAGAGTTCTACA
AGTTCATCAAGCCCATCCTGGAAAAGATGGACGGCACCGAGGAACTGCTCGTGAAGCT
GAACAGAGAGGACCTGCTGCGGAAGCAGCGGACCTTCGACAACGGCAGCATCCCCCA
CCAGATCCACCTGGGAGAGCTGCACGCCATTCTGCGGCGGCAGGAAGATTTTTACCCA
TTCCTGAAGGACAACCGGGAAAAGATCGAGAAGATCCTGACCTTCCGCATCCCCTACT
ACGTGGGCCCTCTGGCCAGGGGAAACAGCAGATTCGCCTGGATGACCAGAAAGAGCG
AGGAAACCATCACCCCCTGGAACTTCGAGGAAGTGGTGGACAAGGGCGCTTCCGCCC
AGAGCTTCATCGAGCGGATGACCAACTTCGATAAGAACCTGCCCAACGAGAAGGTGCT
GCCCAAGCACAGCCTGCTGTACGAGTACTTCACCGTGTATAACGAGCTGACCAAAGTG
AAATACGTGACCGAGGGAATGAGAAAGCCCGCCTTCCTGAGCGGCGAGCAGAAAAAG
GCCATCGTGGACCTGCTGTTCAAGACCAACCGGAAAGTGACCGTGAAGCAGCTGAAAG
AGGACTACTTCAAGAAAATCGAGTGCTTCGACTCCGTGGAAATCTCCGGCGTGGAAGA
TCGGTTCAACGCCTCCCTGGGCACATACCACGATCTGCTGAAAATTATCAAGGACAAG
GACTTCCTGGACAATGAGGAAAACGAGGACATTCTGGAAGATATCGTGCTGACCCTGA
CACTGTTTGAGGACAGAGAGATGATCGAGGAACGGCTGAAAACCTATGCCCACCTGTT
CGACGACAAAGTGATGAAGCAGCTGAAGCGGCGGAGATACACCGGCTGGGGCAGGCT
GAGCCGGAAGCTGATCAACGGCATCCGGGACAAGCAGTCCGGCAAGACAATCCTGGA
TTTCCTGAAGTCCGACGGCTTCGCCAACAGAAACTTCATGCAGCTGATCCACGACGAC
AGCCTGACCTTTAAAGAGGACATCCAGAAAGCCCAGGTGTCCGGCCAGGGCGATAGC
CTGCACGAGCACATTGCCAATCTGGCCGGCAGCCCCGCCATTAAGAAGGGCATCCTGC
AGACAGTGAAGGTGGTGGACGAGCTCGTGAAAGTGATGGGCCGGCACAAGCCCGAGA
ACATCGTGATCGAAATGGCCAGAGAGAACCAGACCACCCAGAAGGGACAGAAGAACAG
CCGCGAGAGAATGAAGCGGATCGAAGAGGGCATCAAAGAGCTGGGCAGCCAGATCCT
GAAAGAACACCCCGTGGAAAACACCCAGCTGCAGAACGAGAAGCTGTACCTGTACTAC
CTGCAGAATGGGCGGGATATGTACGTGGACCAGGAACTGGACATCAACCGGCTGTCC
GACTACGATGTGGACCATATCGTGCCTCAGAGCTTTCTGAAGGACGACTCCATCGACA
ACAAGGTGCTGACCAGAAGCGACAAGAACCGGGGCAAGAGCGACAACGTGCCCTCCG
AAGAGGTCGTGAAGAAGATGAAGAACTACTGGCGGCAGCTGCTGAACGCCAAGCTGAT
TACCCAGAGAAAGTTCGACAATCTGACCAAGGCCGAGAGAGGCGGCCTGAGCGAACT
GGATAAGGCCGGCTTCATCAAGAGACAGCTGGTGGAAACCCGGCAGATCACAAAGCAC
GTGGCACAGATCCTGGACTCCCGGATGAACACTAAGTACGACGAGAATGACAAGCTGA
TCCGGGAAGTGAAAGTGATCACCCTGAAGTCCAAGCTGGTGTCCGATTTCCGGAAGGA
TTTCCAGTTTTACAAAGTGCGCGAGATCAACAACTACCACCACGCCCACGACGCCTACC
TGAACGCCGTCGTGGGAACCGCCCTGATCAAAAAGTACCCTAAGCTGGAAAGCGAGTT
CGTGTACGGCGACTACAAGGTGTACGACGTGCGGAAGATGATCGCCAAGAGCGAGCA
GGAAATCGGCAAGGCTACCGCCAAGTACTTCTTCTACAGCAACATCATGAACTTTTTCA
AGACCGAGATTACCCTGGCCAACGGCGAGATCCGGAAGCGGCCTCTGATCGAGACAA
ACGGCGAAACCGGGGAGATCGTGTGGGATAAGGGCCGGGATTTTGCCACCGTGCGGA
AAGTGCTGAGCATGCCCCAAGTGAATATCGTGAAAAAGACCGAGGTGCAGACAGGCGG
CTTCAGCAAAGAGTCTATCCTGCCCAAGAGGAACAGCGATAAGCTGATCGCCAGAAAG
AAGGACTGGGACCCTAAGAAGTACGGCGGCTTCGACAGCCCCACCGTGGCCTATTCTG
TGCTGGTGGTGGCCAAAGTGGAAAAGGGCAAGTCCAAGAAACTGAAGAGTGTGAAAGA
GCTGCTGGGGATCACCATCATGGAAAGAAGCAGCTTCGAGAAGAATCCCATCGACTTT
CTGGAAGCCAAGGGCTACAAAGAAGTGAAAAAGGACCTGATCATCAAGCTGCCTAAGT
ACTCCCTGTTCGAGCTGGAAAACGGCCGGAAGAGAATGCTGGCCTCTGCCGGCGAAC
TGCAGAAGGGAAACGAACTGGCCCTGCCCTCCAAATATGTGAACTTCCTGTACCTGGC
CAGCCACTATGAGAAGCTGAAGGGCTCCCCCGAGGATAATGAGCAGAAACAGCTGTTT
GTGGAACAGCACAAGCACTACCTGGACGAGATCATCGAGCAGATCAGCGAGTTCTCCA
AGAGAGTGATCCTGGCCGACGCTAATCTGGACAAAGTGCTGTCCGCCTACAACAAGCA
CCGGGATAAGCCCATCAGAGAGCAGGCCGAGAATATCATCCACCTGTTTACCCTGACC
AATCTGGGAGCCCCTGCCGCCTTCAAGTACTTTGACACCACCATCGACCGGAAGAGGT
ACACCAGCACCAAAGAGGTGCTGGACGCCACCCTGATCCACCAGAGCATCACCGGCC
TGTACGAGACACGGATCGACCTGTCTCAGCTGGGAGGTGACtctggcggctcaaaaagaaccgc
cgacggcagcgaattcgagcccaagaagaagaggaaagtcTAA
ABE8.8m
(Gaudelli et al. Nature Biotech. 2020, 38, 892-900)
ABE8.8m (ecTadA*(8.8)-linker(32 aa)-Cas9 nickase-NLS):
lowercase, underlined = linker
CAPS UNDERLINED = evolved ecTadA*
CAPS = Cas9 nickase (D10A mutation underlined)
lowercase = NLS
Protein (SEQ ID NO: 31):
MSEVEFSHEYWMRHALTLAKRARDEREVPVGAVLVLNNRVIGEGWNRAIGLHDPTAHAEI
MALRQGGLVMQNYRLIDATLYVTFEPCVMCAGAMIHSRIGRVVFGVRNAKTGAAGSLMDV
LHHPGMNHRVEITEGILADECAALLCRFFRMPRRVFNAQKKAQSSTDsggssggssgsetpgtses
atpessggssggsDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGDegadkrt
adgsefespkkkrkv*
DNA (SEQ ID NO: 39):
ATGTCCGAAGTCGAGTTTTCCCATGAGTACTGGATGAGACACGCATTGACTCTCGCAAA
GAGGGCTCGAGATGAACGCGAGGTGCCCGTGGGGGCAGTACTCGTGCTCAACAATCG
CGTAATCGGCGAAGGTTGGAATAGGGCAATCGGACTCCACGACCCCACTGCACATGCG
GAAATCATGGCCCTTCGACAGGGAGGGCTTGTGATGCAGAATTATCGACTTATCGATG
CGACGCTGTACGTCACGTTTGAACCTTGCGTAATGTGCGCGGGAGCTATGATTCACTC
CCGCATTGGACGAGTTGTATTCGGTGTTCGCAACGCCAAGACGGGTGCCGCAGGTTCA
CTGATGGACGTGCTGCATCATCCAGGCATGAACCACCGGGTAGAAATCACAGAAGGCA
TATTGGCGGACGAATGTGCGGCGCTGTTGTGTCGTTTTTTTCGCATGCCCAGGCGGGT
CTTTAACGCCCAGAAAAAAGCACAATCCTCTACTGACTCTGGTGGTTCTTCTGGTGGTT
CTAGCGGCAGCGAGACTCCCGGGACCTCAGAGTCCGCCACACCCGAAAGTTCTGGTG
GTTCTTCTGGTGGTTCTGACAAGAAGTACAGCATCGGCCTGGCCATCGGCACCAACTC
TGTGGGCTGGGCCGTGATCACCGACGAGTACAAGGTGCCCAGCAAGAAATTCAAGGT
GCTGGGCAACACCGACCGGCACAGCATCAAGAAGAACCTGATCGGAGCCCTGCTGTT
CGACAGCGGCGAAACAGCCGAGGCCACCCGGCTGAAGAGAACCGCCAGAAGAAGATA
CACCAGACGGAAGAACCGGATCTGCTATCTGCAAGAGATCTTCAGCAACGAGATGGCC
AAGGTGGACGACAGCTTCTTCCACAGACTGGAAGAGTCCTTCCTGGTGGAAGAGGATA
AGAAGCACGAGCGGCACCCCATCTTCGGCAACATCGTGGACGAGGTGGCCTACCACG
AGAAGTACCCCACCATCTACCACCTGAGAAAGAAACTGGTGGACAGCACCGACAAGGC
CGACCTGCGGCTGATCTATCTGGCCCTGGCCCACATGATCAAGTTCCGGGGCCACTTC
CTGATCGAGGGCGACCTGAACCCCGACAACAGCGACGTGGACAAGCTGTTCATCCAG
CTGGTGCAGACCTACAACCAGCTGTTCGAGGAAAACCCCATCAACGCCAGCGGCGTG
GACGCCAAGGCCATCCTGTCTGCCAGACTGAGCAAGAGCAGACGGCTGGAAAATCTGA
TCGCCCAGCTGCCCGGCGAGAAGAAGAATGGCCTGTTCGGAAACCTGATTGCCCTGA
GCCTGGGCCTGACCCCCAACTTCAAGAGCAACTTCGACCTGGCCGAGGATGCCAAACT
GCAGCTGAGCAAGGACACCTACGACGACGACCTGGACAACCTGCTGGCCCAGATCGG
CGACCAGTACGCCGACCTGTTTCTGGCCGCCAAGAACCTGTCCGACGCCATCCTGCTG
AGCGACATCCTGAGAGTGAACACCGAGATCACCAAGGCCCCCCTGAGCGCCTCTATGA
TCAAGAGATACGACGAGCACCACCAGGACCTGACCCTGCTGAAAGCTCTCGTGCGGCA
GCAGCTGCCTGAGAAGTACAAAGAGATTTTCTTCGACCAGAGCAAGAACGGCTACGCC
GGCTACATTGACGGCGGAGCCAGCCAGGAAGAGTTCTACAAGTTCATCAAGCCCATCC
TGGAAAAGATGGACGGCACCGAGGAACTGCTCGTGAAGCTGAACAGAGAGGACCTGC
TGCGGAAGCAGCGGACCTTCGACAACGGCAGCATCCCCCACCAGATCCACCTGGGAG
AGCTGCACGCCATTCTGCGGCGGCAGGAAGATTTTTACCCATTCCTGAAGGACAACCG
GGAAAAGATCGAGAAGATCCTGACCTTCCGCATCCCCTACTACGTGGGCCCTCTGGCC
AGGGGAAACAGCAGATTCGCCTGGATGACCAGAAAGAGCGAGGAAACCATCACCCCCT
GGAACTTCGAGGAAGTGGTGGACAAGGGCGCTTCCGCCCAGAGCTTCATCGAGCGGA
TGACCAACTTCGATAAGAACCTGCCCAACGAGAAGGTGCTGCCCAAGCACAGCCTGCT
GTACGAGTACTTCACCGTGTATAACGAGCTGACCAAAGTGAAATACGTGACCGAGGGA
ATGAGAAAGCCCGCCTTCCTGAGCGGCGAGCAGAAAAAGGCCATCGTGGACCTGCTG
TTCAAGACCAACCGGAAAGTGACCGTGAAGCAGCTGAAAGAGGACTACTTCAAGAAAA
TCGAGTGCTTCGACTCCGTGGAAATCTCCGGCGTGGAAGATCGGTTCAACGCCTCCCT
GGGCACATACCACGATCTGCTGAAAATTATCAAGGACAAGGACTTCCTGGACAATGAG
GAAAACGAGGACATTCTGGAAGATATCGTGCTGACCCTGACACTGTTTGAGGACAGAG
AGATGATCGAGGAACGGCTGAAAACCTATGCCCACCTGTTCGACGACAAAGTGATGAA
GCAGCTGAAGCGGCGGAGATACACCGGCTGGGGCAGGCTGAGCCGGAAGCTGATCAA
CGGCATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTAAAGAG
GACATCCAGAAAGCCCAGGTGTCCGGCCAGGGCGATAGCCTGCACGAGCACATTGCC
AATCTGGCCGGCAGCCCCGCCATTAAGAAGGGCATCCTGCAGACAGTGAAGGTGGTG
GACGAGCTCGTGAAAGTGATGGGCCGGCACAAGCCCGAGAACATCGTGATCGAAATG
GCCAGAGAGAACCAGACCACCCAGAAGGGACAGAAGAACAGCCGCGAGAGAATGAAG
CGGATCGAAGAGGGCATCAAAGAGCTGGGCAGCCAGATCCTGAAAGAACACCCCGTG
GAAAACACCCAGCTGCAGAACGAGAAGCTGTACCTGTACTACCTGCAGAATGGGGGGG
ATATGTACGTGGACCAGGAACTGGACATCAACCGGCTGTCCGACTACGATGTGGACCA
TATCGTGCCTCAGAGCTTTCTGAAGGACGACTCCATCGACAACAAGGTGCTGACCAGA
AGCGACAAGAACCGGGGCAAGAGCGACAACGTGCCCTCCGAAGAGGTCGTGAAGAAG
ATGAAGAACTACTGGCGGCAGCTGCTGAACGCCAAGCTGATTACCCAGAGAAAGTTCG
ACAATCTGACCAAGGCCGAGAGAGGCGGCCTGAGCGAACTGGATAAGGCCGGCTTCA
TCAAGAGACAGCTGGTGGAAACCCGGCAGATCACAAAGCACGTGGCACAGATCCTGGA
CTCCCGGATGAACACTAAGTACGACGAGAATGACAAGCTGATCCGGGAAGTGAAAGTG
ATCACCCTGAAGTCCAAGCTGGTGTCCGATTTCCGGAAGGATTTCCAGTTTTACAAAGT
GCGCGAGATCAACAACTACCACCACGCCCACGACGCCTACCTGAACGCCGTCGTGGG
AACCGCCCTGATCAAAAAGTACCCTAAGCTGGAAAGCGAGTTCGTGTACGGCGACTAC
AAGGTGTACGACGTGCGGAAGATGATCGCCAAGAGCGAGCAGGAAATCGGCAAGGCT
ACCGCCAAGTACTTCTTCTACAGCAACATCATGAACTTTTTCAAGACCGAGATTACCCTG
GCCAACGGCGAGATCCGGAAGCGGCCTCTGATCGAGACAAACGGCGAAACCGGGGAG
ATCGTGTGGGATAAGGGCCGGGATTTTGCCACCGTGCGGAAAGTGCTGAGCATGCCC
CAAGTGAATATCGTGAAAAAGACCGAGGTGCAGACAGGCGGCTTCAGCAAAGAGTCTA
TCCTGCCCAAGAGGAACAGCGATAAGCTGATCGCCAGAAAGAAGGACTGGGACCCTAA
GAAGTACGGCGGCTTCGACAGCCCCACCGTGGCCTATTCTGTGCTGGTGGTGGCCAA
AGTGGAAAAGGGCAAGTCCAAGAAACTGAAGAGTGTGAAAGAGCTGCTGGGGATCACC
ATCATGGAAAGAAGCAGCTTCGAGAAGAATCCCATCGACTTTCTGGAAGCCAAGGGCT
ACAAAGAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAACGAA
CTGGCCCTGCCCTCCAAATATGTGAACTTCCTGTACCTGGCCAGCCACTATGAGAAGCT
GAAGGGCTCCCCCGAGGATAATGAGCAGAAACAGCTGTTTGTGGAACAGCACAAGCAC
TACCTGGACGAGATCATCGAGCAGATCAGCGAGTTCTCCAAGAGAGTGATCCTGGCCG
ACGCTAATCTGGACAAAGTGCTGTCCGCCTACAACAAGCACCGGGATAAGCCCATCAG
AGAGCAGGCCGAGAATATCATCCACCTGTTTACCCTGACCAATCTGGGAGCCCCTGCC
GCCTTCAAGTACTTTGACACCACCATCGACCGGAAGAGGTACACCAGCACCAAAGAGG
TGCTGGACGCCACCCTGATCCACCAGAGCATCACCGGCCTGTACGAGACACGGATCG
ACCTGTCTCAGCTGGGAGGTGACgagggagctgataagcgcaccgccgatggttccgagttcgaaagcccca
agaagaagaggaaagtcTAA
ABE8.13m
(Gaudelli et al. Nature Biotech. 2020, 38, 892-900)
ABE8.13m (ecTadA*(8.13)-linker(32 aa)-Cas9 nickase-NLS):
lowercase, underlined = linker
CAPS UNDERLINED = evolved ecTadA*
CAPS = Cas9 nickase (D10A mutation underlined)
lowercase = NLS
Protein (SEQ ID NO: 32):
MSEVEFSHEYWMRHALTLAKRARDEREVPVGAVLVLNNRVIGEGWNRAIGLHDPTAHAEI
MALRQGGLVMQNYRLYDATLYVTFEPCVMCAGAMIHSRIGRVVFGVRNAKTGAAGSLMDV
LHHPGMNHRVEITEGILADECAALLCRFFRMPRRVFNAQKKAQSSTDsggssggssgsetpgtses
atpessggssggsDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGDegadkrt
adgsefespkkkrkv*
DNA (SEQ ID NO: 40):
ATGTCCGAAGTCGAGTTTTCCCATGAGTACTGGATGAGACACGCATTGACTCTCGCAAA
GAGGGCTCGAGATGAACGCGAGGTGCCCGTGGGGGCAGTACTCGTGCTCAACAATCG
CGTAATCGGCGAAGGTTGGAATAGGGCAATCGGACTCCACGACCCCACTGCACATGCG
GAAATCATGGCCCTTCGACAGGGAGGGCTTGTGATGCAGAATTATCGACTTTATGATGC
GACGCTGTACGTCACGTTTGAACCTTGCGTAATGTGCGCGGGAGCTATGATTCACTCC
CGCATTGGACGAGTTGTATTCGGTGTTCGCAACGCCAAGACGGGTGCCGCAGGTTCAC
TGATGGACGTGCTGCATCATCCAGGCATGAACCACCGGGTAGAAATCACAGAAGGCAT
ATTGGCGGACGAATGTGCGGCGCTGTTGTGTCGTTTTTTTCGCATGCCCAGGGGGGTC
TTTAACGCCCAGAAAAAAGCACAATCCTCTACTGACtctggtggttcttctggtggttctagcggcagcgag
actcccgggacctcagagtccgccacacccgaaagttctggtggttcttctggtggttctGACAAGAAGTACAGCATC
GGCCTGGCCATCGGCACCAACTCTGTGGGCTGGGCCGTGATCACCGACGAGTACAAG
GTGCCCAGCAAGAAATTCAAGGTGCTGGGCAACACCGACCGGCACAGCATCAAGAAGA
ACCTGATCGGAGCCCTGCTGTTCGACAGCGGCGAAACAGCCGAGGCCACCCGGCTGA
AGAGAACCGCCAGAAGAAGATACACCAGACGGAAGAACCGGATCTGCTATCTGCAAGA
GATCTTCAGCAACGAGATGGCCAAGGTGGACGACAGCTTCTTCCACAGACTGGAAGAG
TCCTTCCTGGTGGAAGAGGATAAGAAGCACGAGCGGCACCCCATCTTCGGCAACATCG
TGGACGAGGTGGCCTACCACGAGAAGTACCCCACCATCTACCACCTGAGAAAGAAACT
GGTGGACAGCACCGACAAGGCCGACCTGCGGCTGATCTATCTGGCCCTGGCCCACAT
GATCAAGTTCCGGGGCCACTTCCTGATCGAGGGCGACCTGAACCCCGACAACAGCGA
CGTGGACAAGCTGTTCATCCAGCTGGTGCAGACCTACAACCAGCTGTTCGAGGAAAAC
CCCATCAACGCCAGCGGCGTGGACGCCAAGGCCATCCTGTCTGCCAGACTGAGCAAG
AGCAGACGGCTGGAAAATCTGATCGCCCAGCTGCCCGGCGAGAAGAAGAATGGCCTG
TTCGGAAACCTGATTGCCCTGAGCCTGGGCCTGACCCCCAACTTCAAGAGCAACTTCG
ACCTGGCCGAGGATGCCAAACTGCAGCTGAGCAAGGACACCTACGACGACGACCTGG
ACAACCTGCTGGCCCAGATCGGCGACCAGTACGCCGACCTGTTTCTGGCCGCCAAGAA
CCTGTCCGACGCCATCCTGCTGAGCGACATCCTGAGAGTGAACACCGAGATCACCAAG
GCCCCCCTGAGCGCCTCTATGATCAAGAGATACGACGAGCACCACCAGGACCTGACCC
TGCTGAAAGCTCTCGTGCGGCAGCAGCTGCCTGAGAAGTACAAAGAGATTTTCTTCGA
CCAGAGCAAGAACGGCTACGCCGGCTACATTGACGGCGGAGCCAGCCAGGAAGAGTT
CTACAAGTTCATCAAGCCCATCCTGGAAAAGATGGACGGCACCGAGGAACTGCTCGTG
AAGCTGAACAGAGAGGACCTGCTGCGGAAGCAGCGGACCTTCGACAACGGCAGCATC
CCCCACCAGATCCACCTGGGAGAGCTGCACGCCATTCTGCGGCGGCAGGAAGATTTTT
ACCCATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAGAAA
GAGCGAGGAAACCATCACCCCCTGGAACTTCGAGGAAGTGGTGGACAAGGGCGCTTC
CGCCCAGAGCTTCATCGAGCGGATGACCAACTTCGATAAGAACCTGCCCAACGAGAAG
GTGCTGCCCAAGCACAGCCTGCTGTACGAGTACTTCACCGTGTATAACGAGCTGACCA
AAGTGAAATACGTGACCGAGGGAATGAGAAAGCCCGCCTTCCTGAGCGGCGAGCAGA
AAAAGGCCATCGTGGACCTGCTGTTCAAGACCAACCGGAAAGTGACCGTGAAGCAGCT
GAAAGAGGACTACTTCAAGAAAATCGAGTGCTTCGACTCCGTGGAAATCTCCGGCGTG
GAAGATCGGTTCAACGCCTCCCTGGGCACATACCACGATCTGCTGAAAATTATCAAGGA
CAAGGACTTCCTGGACAATGAGGAAAACGAGGACATTCTGGAAGATATCGTGCTGACC
CTGACACTGTTTGAGGACAGAGAGATGATCGAGGAACGGCTGAAAACCTATGCCCACC
TGTTCGACGACAAAGTGATGAAGCAGCTGAAGCGGCGGAGATACACCGGCTGGGGCA
GGCTGAGCCGGAAGCTGATCAACGGCATCCGGGACAAGCAGTCCGGCAAGACAATCC
TGGATTTCCTGAAGTCCGACGGCTTCGCCAACAGAAACTTCATGCAGCTGATCCACGA
CGACAGCCTGACCTTTAAAGAGGACATCCAGAAAGCCCAGGTGTCCGGCCAGGGCGA
TAGCCTGCACGAGCACATTGCCAATCTGGCCGGCAGCCCCGCCATTAAGAAGGGCATC
CTGCAGACAGTGAAGGTGGTGGACGAGCTCGTGAAAGTGATGGGCCGGCACAAGCCC
GAGAACATCGTGATCGAAATGGCCAGAGAGAACCAGACCACCCAGAAGGGACAGAAG
AACAGCCGCGAGAGAATGAAGCGGATCGAAGAGGGCATCAAAGAGCTGGGCAGCCAG
ATCCTGAAAGAACACCCCGTGGAAAACACCCAGCTGCAGAACGAGAAGCTGTACCTGT
ACTACCTGCAGAATGGGGGGGATATGTACGTGGACCAGGAACTGGACATCAACCGGCT
GTCCGACTACGATGTGGACCATATCGTGCCTCAGAGCTTTCTGAAGGACGACTCCATC
GACAACAAGGTGCTGACCAGAAGCGACAAGAACCGGGGCAAGAGCGACAACGTGCCC
TCCGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAGC
GAACTGGATAAGGCCGGCTTCATCAAGAGACAGCTGGTGGAAACCCGGCAGATCACAA
AGCACGTGGCACAGATCCTGGACTCCCGGATGAACACTAAGTACGACGAGAATGACAA
GCTGATCCGGGAAGTGAAAGTGATCACCCTGAAGTCCAAGCTGGTGTCCGATTTCCGG
AAGGATTTCCAGTTTTACAAAGTGCGCGAGATCAACAACTACCACCACGCCCACGACG
CCTACCTGAACGCCGTCGTGGGAACCGCCCTGATCAAAAAGTACCCTAAGCTGGAAAG
CGAGTTCGTGTACGGCGACTACAAGGTGTACGACGTGCGGAAGATGATCGCCAAGAG
CGAGCAGGAAATCGGCAAGGCTACCGCCAAGTACTTCTTCTACAGCAACATCATGAACT
TTTTCAAGACCGAGATTACCCTGGCCAACGGCGAGATCCGGAAGCGGCCTCTGATCGA
GACAAACGGCGAAACCGGGGAGATCGTGTGGGATAAGGGCCGGGATTTTGCCACCGT
GCGGAAAGTGCTGAGCATGCCCCAAGTGAATATCGTGAAAAAGACCGAGGTGCAGACA
GGCGGCTTCAGCAAAGAGTCTATCCTGCCCAAGAGGAACAGCGATAAGCTGATCGCCA
GAAAGAAGGACTGGGACCCTAAGAAGTACGGCGGCTTCGACAGCCCCACCGTGGCCT
ATTCTGTGCTGGTGGTGGCCAAAGTGGAAAAGGGCAAGTCCAAGAAACTGAAGAGTGT
GAAAGAGCTGCTGGGGATCACCATCATGGAAAGAAGCAGCTTCGAGAAGAATCCCATC
GACTTTCTGGAAGCCAAGGGCTACAAAGAAGTGAAAAAGGACCTGATCATCAAGCTGC
CTAAGTACTCCCTGTTCGAGCTGGAAAACGGCCGGAAGAGAATGCTGGCCTCTGCCGG
CGAACTGCAGAAGGGAAACGAACTGGCCCTGCCCTCCAAATATGTGAACTTCCTGTAC
CTGGCCAGCCACTATGAGAAGCTGAAGGGCTCCCCCGAGGATAATGAGCAGAAACAG
CTGTTTGTGGAACAGCACAAGCACTACCTGGACGAGATCATCGAGCAGATCAGCGAGT
TCTCCAAGAGAGTGATCCTGGCCGACGCTAATCTGGACAAAGTGCTGTCCGCCTACAA
CAAGCACCGGGATAAGCCCATCAGAGAGCAGGCCGAGAATATCATCCACCTGTTTACC
CTGACCAATCTGGGAGCCCCTGCCGCCTTCAAGTACTTTGACACCACCATCGACCGGA
AGAGGTACACCAGCACCAAAGAGGTGCTGGACGCCACCCTGATCCACCAGAGCATCAC
CGGCCTGTACGAGACACGGATCGACCTGTCTCAGCTGGGAGGTGACgagggagctgataagc
gcaccgccgatggttccgagttcgaaagccccaagaagaagaggaaagtcTAA
ABE8.17m
(Gaudelli et al. Nature Biotech. 2020, 38, 892-900)
ABE8.17m (ecTadA*(8.17)-linker(32 aa)-Cas9 nickase-NLS):
lowercase, underlined = linker
CAPS UNDERLINED = evolved ecTadA*
CAPS = Cas9 nickase (D10A mutation underlined)
lowercase = NLS
Protein (SEQ ID NO: 33):
MSEVEFSHEYWMRHALTLAKRARDEREVPVGAVLVLNNRVIGEGWNRAIGLHDPTAHAEI
MALRQGGLVMQNYRLIDATLYSTFEPCVMCAGAMIHSRIGRVVFGVRNAKTGAAGSLMDV
LHYPGMNHRVEITEGILADECAALLCYFFRMPRRVFNAQKKAQSSTDsggssggssgsetpgtses
atpessggssggsDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQ
EEFYKFIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLIGITIMERSSFEKNPIDFL
EAKGYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGDegadkrt
adgsefespkkkrkv*
DNA (SEQ ID NO: 41):
ATGTCCGAAGTCGAGTTTTCCCATGAGTACTGGATGAGACACGCATTGACTCTCGCAAA
GAGGGCTCGAGATGAACGCGAGGTGCCCGTGGGGGCAGTACTCGTGCTCAACAATCG
CGTAATCGGCGAAGGTTGGAATAGGGCAATCGGACTCCACGACCCCACTGCACATGCG
GAAATCATGGCCCTTCGACAGGGAGGGCTTGTGATGCAGAATTATCGACTTATCGATG
CGACGCTGTACTCGACGTTTGAACCTTGCGTAATGTGCGCGGGAGCTATGATTCACTC
CCGCATTGGACGAGTTGTATTCGGTGTTCGCAACGCCAAGACGGGTGCCGCAGGTTCA
CTGATGGACGTGCTGCATTACCCAGGCATGAACCACCGGGTAGAAATCACAGAAGGCA
TATTGGCGGACGAATGTGCGGCGCTGTTGTGTTACTTTTTTCGCATGCCCAGGCGTGT
CTTTAACGCCCAGAAAAAAGCACAATCCTCTACTGACtctggtggttcttctggtggttctagcggcagcg
agactcccgggacctcagagtccgccacacccgaaagttctggtggttcttctggtggttctGACAAGAAGTACAGCAT
CGGCCTGGCCATCGGCACCAACTCTGTGGGCTGGGCCGTGATCACCGACGAGTACAA
GGTGCCCAGCAAGAAATTCAAGGTGCTGGGCAACACCGACCGGCACAGCATCAAGAA
GAACCTGATCGGAGCCCTGCTGTTCGACAGCGGCGAAACAGCCGAGGCCACCCGGCT
GAAGAGAACCGCCAGAAGAAGATACACCAGACGGAAGAACCGGATCTGCTATCTGCAA
GAGATCTTCAGCAACGAGATGGCCAAGGTGGACGACAGCTTCTTCCACAGACTGGAAG
AGTCCTTCCTGGTGGAAGAGGATAAGAAGCACGAGCGGCACCCCATCTTCGGCAACAT
CGTGGACGAGGTGGCCTACCACGAGAAGTACCCCACCATCTACCACCTGAGAAAGAAA
CTGGTGGACAGCACCGACAAGGCCGACCTGCGGCTGATCTATCTGGCCCTGGCCCAC
ATGATCAAGTTCCGGGGCCACTTCCTGATCGAGGGCGACCTGAACCCCGACAACAGCG
ACGTGGACAAGCTGTTCATCCAGCTGGTGCAGACCTACAACCAGCTGTTCGAGGAAAA
CCCCATCAACGCCAGCGGCGTGGACGCCAAGGCCATCCTGTCTGCCAGACTGAGCAA
GAGCAGACGGCTGGAAAATCTGATCGCCCAGCTGCCCGGCGAGAAGAAGAATGGCCT
GTTCGGAAACCTGATTGCCCTGAGCCTGGGCCTGACCCCCAACTTCAAGAGCAACTTC
GACCTGGCCGAGGATGCCAAACTGCAGCTGAGCAAGGACACCTACGACGACGACCTG
GACAACCTGCTGGCCCAGATCGGCGACCAGTACGCCGACCTGTTTCTGGCCGCCAAG
AACCTGTCCGACGCCATCCTGCTGAGCGACATCCTGAGAGTGAACACCGAGATCACCA
AGGCCCCCCTGAGCGCCTCTATGATCAAGAGATACGACGAGCACCACCAGGACCTGAC
CCTGCTGAAAGCTCTCGTGCGGCAGCAGCTGCCTGAGAAGTACAAAGAGATTTTCTTC
GACCAGAGCAAGAACGGCTACGCCGGCTACATTGACGGCGGAGCCAGCCAGGAAGAG
TTCTACAAGTTCATCAAGCCCATCCTGGAAAAGATGGACGGCACCGAGGAACTGCTCG
TGAAGCTGAACAGAGAGGACCTGCTGCGGAAGCAGCGGACCTTCGACAACGGCAGCA
TCCCCCACCAGATCCACCTGGGAGAGCTGCACGCCATTCTGCGGCGGCAGGAAGATTT
TTACCCATTCCTGAAGGACAACCGGGAAAAGATCGAGAAGATCCTGACCTTCCGCATC
CCCTACTACGTGGGCCCTCTGGCCAGGGGAAACAGCAGATTCGCCTGGATGACCAGA
AAGAGCGAGGAAACCATCACCCCCTGGAACTTCGAGGAAGTGGTGGACAAGGGCGCT
TCCGCCCAGAGCTTCATCGAGCGGATGACCAACTTCGATAAGAACCTGCCCAACGAGA
AGGTGCTGCCCAAGCACAGCCTGCTGTACGAGTACTTCACCGTGTATAACGAGCTGAC
CAAAGTGAAATACGTGACCGAGGGAATGAGAAAGCCCGCCTTCCTGAGCGGCGAGCA
GAAAAAGGCCATCGTGGACCTGCTGTTCAAGACCAACCGGAAAGTGACCGTGAAGCAG
CTGAAAGAGGACTACTTCAAGAAAATCGAGTGCTTCGACTCCGTGGAAATCTCCGGCG
TGGAAGATCGGTTCAACGCCTCCCTGGGCACATACCACGATCTGCTGAAAATTATCAAG
GACAAGGACTTCCTGGACAATGAGGAAAACGAGGACATTOTGGAAGATATCGTGCTGA
CCCTGACACTGTTTGAGGACAGAGAGATGATCGAGGAACGGCTGAAAACCTATGCCCA
CCTGTTCGACGACAAAGTGATGAAGCAGCTGAAGCGGCGGAGATACACCGGCTGGGG
CAGGCTGAGCCGGAAGCTGATCAACGGCATCCGGGACAAGCAGTCCGGCAAGACAAT
CCTGGATTTCCTGAAGTCCGACGGCTTCGCCAACAGAAACTTCATGCAGCTGATCCAC
GACGACAGCCTGACCTTTAAAGAGGACATCCAGAAAGCCCAGGTGTCCGGCCAGGGC
GATAGCCTGCACGAGCACATTGCCAATCTGGCCGGCAGCCCCGCCATTAAGAAGGGC
ATCCTGCAGACAGTGAAGGTGGTGGACGAGCTCGTGAAAGTGATGGGCCGGCACAAG
CCCGAGAACATCGTGATCGAAATGGCCAGAGAGAACCAGACCACCCAGAAGGGACAG
AAGAACAGCCGCGAGAGAATGAAGCGGATCGAAGAGGGCATCAAAGAGCTGGGCAGC
CAGATCCTGAAAGAACACCCCGTGGAAAACACCCAGCTGCAGAACGAGAAGCTGTACC
TGTACTACCTGCAGAATGGGCGGGATATGTACGTGGACCAGGAACTGGACATCAACCG
GCTGTCCGACTACGATGTGGACCATATCGTGCCTCAGAGCTTTCTGAAGGACGACTCC
ATCGACAACAAGGTGCTGACCAGAAGCGACAAGAACCGGGGCAAGAGCGACAACGTG
CCCTCCGAAGAGGTCGTGAAGAAGATGAAGAACTACTGGCGGCAGCTGCTGAACGCC
AAGCTGATTACCCAGAGAAAGTTCGACAATCTGACCAAGGCCGAGAGAGGCGGCCTGA
GCGAACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGAATGA
CAAGCTGATCCGGGAAGTGAAAGTGATCACCCTGAAGTCCAAGCTGGTGTCCGATTTC
CGGAAGGATTTCCAGTTTTACAAAGTGCGCGAGATCAACAACTACCACCACGCCCACG
ACGCCTACCTGAACGCCGTCGTGGGAACCGCCCTGATCAAAAAGTACCCTAAGCTGGA
AAGCGAGTTCGTGTACGGCGACTACAAGGTGTACGACGTGCGGAAGATGATCGCCAAG
AGCGAGCAGGAAATOGGCAAGGCTACCGCCAAGTACTTCTTCTACAGCAACATCATGA
ACTTTTTCAAGACCGAGATTACCCTGGCCAACGGCGAGATCCGGAAGCGGCCTCTGAT
CGAGACAAACGGCGAAACCGGGGAGATCGTGTGGGATAAGGGCCGGGATTTTGCCAC
CGTGCGGAAAGTGCTGAGCATGCCCCAAGTGAATATCGTGAAAAAGACCGAGGTGCAG
ACAGGCGGCTTCAGCAAAGAGTCTATCCTGCCCAAGAGGAACAGCGATAAGOTGATCG
CCAGAAAGAAGGACTGGGACCCTAAGAAGTACGGCGGCTTCGACAGCCCCACCGTGG
CCTATTCTGTGCTGGTGGTGGCCAAAGTGGAAAAGGGCAAGTCCAAGAAACTGAAGAG
TGTGAAAGAGCTGCTGGGGATCACCATCATGGAAAGAAGCAGCTTCGAGAAGAATCCC
ATCGACTTTCTGGAAGCCAAGGGCTACAAAGAAGTGAAAAAGGACCTGATCATCAAGCT
GCCTAAGTACTCCCTGTTCGAGCTGGAAAACGGCCGGAAGAGAATGCTGGCCTCTGCC
GGCGAACTGCAGAAGGGAAACGAACTGGCCCTGCCCTCCAAATATGTGAACTTCCTGT
ACCTGGCCAGCCACTATGAGAAGCTGAAGGGCTCCCCCGAGGATAATGAGCAGAAACA
GCTGTTTGTGGAACAGCACAAGCACTACCTGGACGAGATCATCGAGCAGATCAGCGAG
TTCTCCAAGAGAGTGATCCTGGCCGACGCTAATCTGGACAAAGTGCTGTCCGCCTACA
ACAAGCACCGGGATAAGCCCATCAGAGAGCAGGCCGAGAATATCATCCACCTGTTTAC
CCTGACCAATCTGGGAGCCCCTGCCGCCTTCAAGTACTTTGACACCACCATCGACCGG
AAGAGGTACACCAGCACCAAAGAGGTGCTGGACGCCACCCTGATCCACCAGAGCATCA
CCGGCCTGTACGAGACACGGATCGACCTGTCTCAGCTGGGAGGTGACgagggagctgataa
gcgcaccgccgatggttccgagttcgaaagccccaagaagaagaggaaagtcTAA
ABE8.20m
(Gaudelli et al. Nature Biotech. 2020, 38, 892-900)
ABE8.20m (ecTadA*(8.20)-linker(32 aa)-Cas9 nickase-NLS):
lowercase, underlined = linker
CAPS UNDERLINED = evolved ecTadA*
CAPS = Cas9 nickase (D10A mutation underlined)
lowercase = NLS
Protein (SEQ ID NO: 34):
MSEVEFSHEYWMRHALTLAKRARDEREVPVGAVLVLNNRVIGEGWNRAIGLHDPTAHAEI
MALRQGGLVMQNYRLYDATLYSTFEPCVMCAGAMIHSRIGRVVFGVRNAKTGAAGSLMDV
LHHPGMNHRVEITEGILADECAALLCRFFRMPRRVFNAQKKAQSSTDsggssggssgsetpgtses
atpessggssggsDKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDKKHER
HPIFGNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFGNLIA
LSLGLTPNFKSNFDLAEDAKLQLSKDTYDDDLDNLLAQIGDQYADLFLAAKNLSDAILLSDILR
VNTEITKAPLSASMIKRYDEHHQDLTLLKALVRQQLPEKYKEIFFDQSKNGYAGYIDGGASQ
EEFYKFIKPILEKMDGTEELLVKLNREDLLRKORTFDNGSIPHQIHLGELHAILRRQEDFYPFL
KDNREKIEKILTFRIPYYVGPLARGNSRFAWMTRKSEETITPWNFEEVVDKGASAQSFIERM
TNFDKNLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENEDILEDIVLT
LTLFEDREMIEERLKTYAHLFDDKVMKQLKRRRYTGWGRLSRKLINGIRDKQSGKTILDFLK
SDGFANRNFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPVENTQLQ
NEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANGEI
RKRPLIETNGETGEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIMERSSFEKNPIDFL
EAKGYKEVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPIREQAEN
IIHLFTLTNLGAPAAFKYFDTTIDRKRYTSTKEVLDATLIHQSITGLYETRIDLSQLGGDegadkrt
adgsefespkkkrkv*
DNA (SEQ ID NO: 42):
ATGTCCGAAGTCGAGTTTTCCCATGAGTACTGGATGAGACACGCATTGACTCTCGCAAA
GAGGGCTCGAGATGAACGCGAGGTGCCCGTGGGGGCAGTACTCGTGCTCAACAATCG
CGTAATCGGCGAAGGTTGGAATAGGGCAATCGGACTCCACGACCCCACTGCACATGCG
GAAATCATGGCCCTTCGACAGGGAGGGCTTGTGATGCAGAATTATCGACTTTATGATGC
GACGCTGTACTCGACGTTTGAACCTTGCGTAATGTGCGCGGGAGCTATGATTCACTCC
CGCATTGGACGAGTTGTATTCGGTGTTCGCAACGCCAAGACGGGTGCCGCAGGTTCAC
TGATGGACGTGCTGCATCATCCAGGCATGAACCACCGGGTAGAAATCACAGAAGGCAT
ATTGGCGGACGAATGTGCGGCGCTGTTGTGTCGTTTTTTTCGCATGCCCAGGCGGGTC
TTTAACGCCCAGAAAAAAGCACAATCCTCTACTGACtctggtggttcttctggggttctagcggcagcgag
actcccgggacctcagagtccgccacacccgaaagttctggtggttcttctggtggttctGACAAGAAGTACAGCATC
GGCCTGGCCATCGGCACCAACTCTGTGGGCTGGGCCGTGATCACCGACGAGTACAAG
GTGCCCAGCAAGAAATTCAAGGTGCTGGGCAACACCGACCGGCACAGCATCAAGAAGA
ACCTGATCGGAGCCCTGCTGTTCGACAGCGGCGAAACAGCCGAGGCCACCCGGCTGA
AGAGAACCGCCAGAAGAAGATACACCAGACGGAAGAACCGGATCTGCTATCTGCAAGA
GATCTTCAGCAACGAGATGGCCAAGGTGGACGACAGCTTCTTCCACAGACTGGAAGAG
TCCTTCCTGGTGGAAGAGGATAAGAAGCACGAGCGGCACCCCATCTTCGGCAACATCG
TGGACGAGGTGGCCTACCACGAGAAGTACCCCACCATCTACCACCTGAGAAAGAAACT
GGTGGACAGCACCGACAAGGCCGACCTGCGGCTGATCTATCTGGCCCTGGCCCACAT
GATCAAGTTCCGGGGCCACTTCCTGATCGAGGGCGACCTGAACCCCGACAACAGCGA
CGTGGACAAGCTGTTCATCCAGCTGGTGCAGACCTACAACCAGCTGTTCGAGGAAAAC
CCCATCAACGCCAGCGGCGTGGACGCCAAGGCCATCCTGTCTGCCAGACTGAGCAAG
AGCAGACGGCTGGAAAATCTGATCGCCCAGCTGCCCGGCGAGAAGAAGAATGGCCTG
TTCGGAAACCTGATTGCCCTGAGCCTGGGCCTGACCCCCAACTTCAAGAGCAACTTCG
ACCTGGCCGAGGATGCCAAACTGCAGCTGAGCAAGGACACCTACGACGACGACCTGG
ACAACCTGCTGGCCCAGATCGGCGACCAGTACGCCGACCTGTTTCTGGCCGCCAAGAA
CCTGTCCGACGCCATCCTGCTGAGCGACATCCTGAGAGTGAACACCGAGATCACCAAG
GCCCCCCTGAGCGCCTCTATGATCAAGAGATACGACGAGCACCACCAGGACCTGACCC
TGCTGAAAGCTCTCGTGCGGCAGCAGCTGCCTGAGAAGTACAAAGAGATTTTCTTCGA
CCAGAGCAAGAACGGCTACGCCGGCTACATTGACGGCGGAGCCAGCCAGGAAGAGTT
CTACAAGTTCATCAAGCCCATCCTGGAAAAGATGGACGGCACCGAGGAACTGCTCGTG
AAGCTGAACAGAGAGGACCTGCTGCGGAAGCAGCGGACCTTCGACAACGGCAGCATC
CCCCACCAGATCCACCTGGGAGAGCTGCACGCCATTCTGCGGCGGCAGGAAGATTTTT
ACCCATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAGAAA
GAGCGAGGAAACCATCACCCCCTGGAACTTCGAGGAAGTGGTGGACAAGGGCGCTTC
CGCCCAGAGCTTCATCGAGCGGATGACCAACTTCGATAAGAACCTGCCCAACGAGAAG
GTGCTGCCCAAGCACAGCCTGCTGTACGAGTACTTCACCGTGTATAACGAGCTGACCA
AAGTGAAATACGTGACCGAGGGAATGAGAAAGCCCGCCTTCCTGAGCGGCGAGCAGA
AAAAGGCCATCGTGGACCTGCTGTTCAAGACCAACCGGAAAGTGACCGTGAAGCAGCT
GAAAGAGGACTACTTCAAGAAAATCGAGTGCTTCGACTCCGTGGAAATCTCCGGCGTG
GAAGATCGGTTCAACGCCTCCCTGGGCACATACCACGATCTGCTGAAAATTATCAAGGA
CAAGGACTTCCTGGACAATGAGGAAAACGAGGACATTCTGGAAGATATCGTGCTGACC
CTGACACTGTTTGAGGACAGAGAGATGATCGAGGAACGGCTGAAAACCTATGCCCACC
TGTTCGACGACAAAGTGATGAAGCAGCTGAAGCGGCGGAGATACACCGGCTGGGGCA
GGCTGAGCCGGAAGCTGATCAACGGCATCCGGGACAAGCAGTCCGGCAAGACAATCC
TGGATTTCCTGAAGTCCGACGGCTTCGCCAACAGAAACTTCATGCAGCTGATCCACGA
CGACAGCCTGACCTTTAAAGAGGACATCCAGAAAGCCCAGGTGTCCGGCCAGGGCGA
TAGCCTGCACGAGCACATTGCCAATCTGGCCGGCAGCCCCGCCATTAAGAAGGGCATC
CTGCAGACAGTGAAGGTGGTGGACGAGCTCGTGAAAGTGATGGGCCGGCACAAGCCC
GAGAACATCGTGATCGAAATGGCCAGAGAGAACCAGACCACCCAGAAGGGACAGAAG
AACAGCCGCGAGAGAATGAAGCGGATCGAAGAGGGCATCAAAGAGCTGGGCAGCCAG
ATCCTGAAAGAACACCCCGTGGAAAACACCCAGCTGCAGAACGAGAAGCTGTACCTGT
ACTACCTGCAGAATGGGGGGGATATGTACGTGGACCAGGAACTGGACATCAACCGGCT
GTCCGACTACGATGTGGACCATATCGTGCCTCAGAGCTTTCTGAAGGACGACTCCATC
GACAACAAGGTGCTGACCAGAAGCGACAAGAACCGGGGCAAGAGCGACAACGTGCCC
TCCGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAGC
GAACTGGATAAGGCCGGCTTCATCAAGAGACAGCTGGTGGAAACCCGGCAGATCACAA
AGCACGTGGCACAGATCCTGGACTCCCGGATGAACACTAAGTACGACGAGAATGACAA
GCTGATCCGGGAAGTGAAAGTGATCACCCTGAAGTCCAAGCTGGTGTCCGATTTCCGG
AAGGATTTCCAGTTTTACAAAGTGCGCGAGATCAACAACTACCACCACGCCCACGACG
CCTACCTGAACGCCGTCGTGGGAACCGCCCTGATCAAAAAGTACCCTAAGCTGGAAAG
CGAGTTCGTGTACGGCGACTACAAGGTGTACGACGTGCGGAAGATGATCGCCAAGAG
CGAGCAGGAAATCGGCAAGGCTACCGCCAAGTACTTCTTCTACAGCAACATCATGAACT
TTTTCAAGACCGAGATTACCCTGGCCAACGGCGAGATCCGGAAGCGGCCTCTGATCGA
GACAAACGGCGAAACCGGGGAGATCGTGTGGGATAAGGGCCGGGATTTTGCCACCGT
GCGGAAAGTGCTGAGCATGCCCCAAGTGAATATCGTGAAAAAGACCGAGGTGCAGACA
GGCGGCTTCAGCAAAGAGTCTATCCTGCCCAAGAGGAACAGCGATAAGCTGATCGCCA
GAAAGAAGGACTGGGACCCTAAGAAGTACGGCGGCTTCGACAGCCCCACCGTGGCCT
ATTCTGTGCTGGTGGTGGCCAAAGTGGAAAAGGGCAAGTCCAAGAAACTGAAGAGTGT
GAAAGAGCTGCTGGGGATCACCATCATGGAAAGAAGCAGCTTCGAGAAGAATCCCATC
GACTTTCTGGAAGCCAAGGGCTACAAAGAAGTGAAAAAGGACCTGATCATCAAGCTGC
CTAAGTACTCCCTGTTCGAGCTGGAAAACGGCCGGAAGAGAATGCTGGCCTCTGCCGG
CGAACTGCAGAAGGGAAACGAACTGGCCCTGCCCTCCAAATATGTGAACTTCCTGTAC
CTGGCCAGCCACTATGAGAAGCTGAAGGGCTCCCCCGAGGATAATGAGCAGAAACAG
CTGTTTGTGGAACAGCACAAGCACTACCTGGACGAGATCATCGAGCAGATCAGCGAGT
TCTCCAAGAGAGTGATCCTGGCCGACGCTAATCTGGACAAAGTGCTGTCCGCCTACAA
CAAGCACCGGGATAAGCCCATCAGAGAGCAGGCCGAGAATATCATCCACCTGTTTACC
CTGACCAATCTGGGAGCCCCTGCCGCCTTCAAGTACTTTGACACCACCATCGACCGGA
AGAGGTACACCAGCACCAAAGAGGTGCTGGACGCCACCCTGATCCACCAGAGCATCAC
CGGCCTGTACGAGACACGGATCGACCTGTCTCAGCTGGGAGGTGACgagggagctgataagc
gcaccgccgatggttccgagttcgaaagccccaagaagaagaggaaagtcTAA
SEQ ID NO: 43
DNA encoding g04 gRNA
gttcctgtaagataccaaa
SEQ ID NO: 44
g04 gRNA
guuccuguaagauaccaaa
SEQ ID NO: 45
ABE ecTadA wild-type, protein
SEVEFSHEYWMRHALTLAKRAWDEREVPVGAVLVHNNRVIGEGWNRPIGRHDPTAHAEIMALRQGGLV
MQNYRLIDATLYVTLEPCVMCAGAMIHSRIGRVVFGARDAKTGAAGSLMDVLHHPGMNHRVEITEGIL
ADECAALLSDFFRMRRQEIKAQKKAQSSTD
SEQ ID NO: 46
ABE ecTadA*7.9, protein
SEVEFSHEYWMRHALTLAKRALDEREVPVGAVLVLNNRVIGEGWNRAIGLHDPTAHAEIMALRQGGLV
MQNYRLIDATLYVTFEPCVMCAGAMIHSRIGRVVFGVRNAKTGAAGSLMDVLHYPGMNHRVEITEGIL
ADECNALLCYFFRMPRQVFNAQKKAQSSTD
SEQ ID NO: 47
ABE ecTadA*7.10, protein
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNAKTGAAGSLMDVLHYPGMNHRVEITEGIL
ADECAALLCYFFRMPRQVFNAQKKAQSSTD
SEQ ID NO: 48
ABE ecTadA*8e, protein
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNSKRGAAGSLMNVLNYPGMNHRVEITEGIL
ADECAALLCDFYRMPRQVFNAQKKAQSSIN
SEQ ID NO: 49
ABE ecTadA*8.8, protein
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNAKTGAAGSLMDVLHHPGMNHRVEITEGIL
ADECAALLCRFFRMPRRVFNAQKKAQSSTD
SEQ ID NO: 50
ABE ecTadA*8.13, protein
SEVEFSHEYWMRHALTLAKRARDEREVPVGAVLVLNNRVIGEGWNRAIGLHDPTAHAEIMALRQGGLV
MQNYRLYDATLYVTFEPCVMCAGAMIHSRIGRVVFGVRNAKTGAAGSLMDVLHHPGMNHRVEITEGIL
ADECAALLCRFFRMPRRVFNAQKKAQSSTD
SEQ ID NO: 51
ABE ecTadA*8.17, protein
SEVEFSHEYWMRHALTLAKRARDEREVPVGAVLVLNNRVIGEGWNRAIGLHDPTAHAEIMALRQGGLV
MQNYRLIDATLYSTFEPCVMCAGAMIHSRIGRVVFGVRNAKTGAAGSLMDVLHYPGMNHRVEITEGIL
ADECAALLCYFFRMPRRVFNAQKKAQSSTD
SEQ ID NO: 52
ABE ecTadA*8.20, protein
ecTadA*8.20
SEVEFSHEYWMRHALTLAKRARDEREVPVGAVLVLNNRVIGEGWNRAIGLHDPTAHAEIMALRQGGLV
MQNYRLYDATLYSTFEPCVMCAGAMIHSRIGRVVFGVRNAKTGAAGSLMDVLHHPGMNHRVEITEGIL
ADECAALLCRFFRMPRRVFNAQKKAQSSTD
SEQ ID NO: 53
ABE ecTadA wild-type, DNA
tctgaagtcgagtttagccacgagtattggatgaggcacgcactgaccctggcaaagcgagcatggga
tgaaagagaagtccccgtgggcgccgtgctggtgcacaacaatagagtgatcggagagggatggaaca
ggccaatcggccgccacgaccctaccgcacacgcagagatcatggcactgaggcagggaggcctggtc
atgcagaattaccgcctgatcgatgccaccctgtatgtgacactggagccatgcgtgatgtgcgcagg
agcaatgatccacagcaggatcggaagagtggtgttcggagcacgggacgccaagaccggcgcagcag
gctccctgatggatgtgctgcaccaccccggcatgaaccaccgggtggagatcacagagggaatcctg
gcagacgagtgcgccgccctgctgagcgatttctttagaatgcggagacaggagatcaaggcccagaa
gaaggcacagagctccaccgac
SEQ ID NO: 54
ABE ecTadA*7.9, DNA
tccgaagtcgagttttcccatgagtactggatgagacacgcattgactctcgcaaagagggctctcga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcattacccaggcatgaaccaccgggtagaaatcacagaaggcatattg
gcggacgaatgtaacgcgctgttgtgttacttttttcgcatgcccaggcaggtctttaacgcccagaa
aaaagcacaatcctctactgac
SEQ ID NO: 55
ABE ecTadA*7.10, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcattacccaggcatgaaccaccgggtagaaatcacagaaggcatattg
gcggacgaatgtgcggcgctgttgtgttacttttttcgcatgcccaggcaggtctttaacgcccagaa
aaaagcacaatcctctactgac
SEQ ID NO: 56
ABE ecTadA*8e, DNA
tctgaggtggagttttcccacgagtactggatgagacatgccctgaccctggccaagagggcacggga
tgagagggaggtgcctgtgggagccgtgctggtgctgaacaatagagtgatcggcgagggctggaaca
gagccatcggcctgcacgacccaacagcccatgccgaaattatggccctgagacagggcggcctggtc
atgcagaactacagactgattgacgccaccctgtacgtgacattcgagccttgcgtgatgtgcgccgg
cgccatgatccactctaggatcggccgcgtggtgtttggcgtgaggaactcaaaaagaggcgccgcag
gctccctgatgaacgtgctgaactaccccggcatgaatcaccgcgtcgaaattaccgagggaatcctg
gcagatgaatgtgccgccctgctgtgcgatttctatcggatgcctagacaggtgttcaatgctcagaa
gaaggcccagagctccatcaac
SEQ ID NO: 57
ABE ecTadA*8.8, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcatcatccaggcatgaaccaccgggtagaaatcacagaaggcatattg
gcggacgaatgtgcggcgctgttgtgtcgtttttttcgcatgcccaggcgggtctttaacgcccagaa
aaaagcacaatcctctactgactctggtggttcttctggtggttctagcggcagcgagactcccggga
cctcagagtccgccacacccgaaagttctggtggttcttctggtggttct
SEQ ID NO: 58
ABE ecTadA*8.13, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgactttatgatgcgacgctgtacgtcacgtttgaaccttgcgtaatgtgcgcggg
agctatgattcactcccgcattggacgagttgtattcggtgttcgcaacgccaagacgggtgccgcag
gttcactgatggacgtgctgcatcatccaggcatgaaccaccgggtagaaatcacagaaggcatattg
gcggacgaatgtgcggcgctgttgtgtcgtttttttcgcatgcccaggcgggtctttaacgcccagaa
aaaagcacaatcctctactgac
SEQ ID NO: 59
ABE ecTadA*8.17, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgacttatcgatgcgacgctgtactcgacgtttgaaccttgcgtaatgtgcgcggg
agctatgattcactcccgcattggacgagttgtattcggtgttcgcaacgccaagacgggtgccgcag
gttcactgatggacgtgctgcattacccaggcatgaaccaccgggtagaaatcacagaaggcatattg
gcggacgaatgtgcggcgctgttgtgttacttttttcgcatgcccaggcgtgtctttaacgcccagaa
aaaagcacaatcctctactgac
SEQ ID NO: 60
ABE ecTadA*8.20, DNA
tccgaagtcgagttttcccatgagtactggatgagacacgcattgactctcgcaaagagggctcgaga
tgaacgcgaggtgcccgtgggggcagtactcgtgctcaacaatcgcgtaatcggcgaaggttggaata
gggcaatcggactccacgaccccactgcacatgcggaaatcatggcccttcgacagggagggcttgtg
atgcagaattatcgactttatgatgcgacgctgtactcgacgtttgaaccttgcgtaatgtgcgcggg
agctatgattcactcccgcattggacgagttgtattcggtgttcgcaacgccaagacgggtgccgcag
gttcactgatggacgtgctgcatcatccaggcatgaaccaccgggtagaaatcacagaaggcatattg
gcggacgaatgtgcggcgctgttgtgtcgtttttttcgcatgcccaggcgggtctttaacgcccagaa
aaaagcacaatcctctactgac
SEQ ID NO: 61
Linker, amino acid
SGGSSGGSSGSETPGTSESATPESSGGSSGGS
SEQ ID NO: 62
Linker, amino acid
SGGS
SEQ ID NO: 63
Linker, DNA
tctggtggttcttctggtggttctagcggcagcgagactcccgggacctcagagtccgccacacccga
aagttctggtggttcttctggtggttct
SEQ ID NO: 64
Linker, DNA
tctggtggttct
SEQ ID NO: 65
NLS, amino acid
PKKKRKV
SEQ ID NO: 66
NLS, amino acid
KRTADGSEFEPKKKRKV
SEQ ID NO: 67
NLS, amino acid
KRTADGSEFESPKKKRKV
SEQ ID NO: 68
NLS, amino acid
EGADKRTADGSEFESPKKKRKV
SEQ ID NO: 69
NLS, DNA
ccc aag aag aag agg aaa gtc
SEQ ID NO: 70
NLS, DNA
aaa aga acc gcc gac ggc agc gaa ttc gag ccc aag aag aag agg aaa
gtc
SEQ ID NO: 71
NLS, DNA
aaa cgg aca gcc gac gga agc gag ttc gag tca cca aag aag aag cgg
aaa gtc
SEQ ID NO: 72
NLS, DNA
gag gga gct gat aag cgc acc gcc gat ggt tcc gag ttc gaa agc ccc
aag aag aag agg aaa gtc
SEQ ID NO: 73
DNA sequence of the gRNA constant region
gtttaagagctatgctggaaacagcatagcaagtttaaataaggctagtccgttatcaactt
gaaaaagtggcaccgagtcggtgc
SEQ ID NO: 74
RNA sequence of the gRNA constant region
Guuuaagagcuaugcuggaaacagcauagcaaguuuaaauaaggcuaguccguuaucaacuu
gaaaaaguggcaccgagucggugc

1. A CRISPR/Cas-based base editing system for altering an RNA splice site encoded in the genomic DNA of a subject, the CRISPR/Cas-based base editing system comprising a fusion protein and at least one guide RNA (gRNA), wherein the fusion protein comprises a Cas protein and a base-editing domain, and
wherein the at least one gRNA targets a sequence comprising at least one of SEQ ID NOs: 21-23 or 43 or a complement or a fragment thereof and/or the gRNA comprises a sequence selected from SEQ ID NOs: 24-26 or 44 or a complement or a fragment thereof.

2. A CRISPR/Cas-based base editing system for altering an RNA splice site encoded in the genomic DNA of a subject, the CRISPR/Cas-based base editing system comprising a fusion protein and at least one guide RNA (gRNA), wherein the fusion protein comprises a Cas protein and a base-editing domain, and
wherein the base-editing domain comprises a polypeptide selected from SEQ ID NOs: 45-52 and/or is encoded by a polynucleotide comprising a sequence selected from SEQ ID NOs: 53-80.

3. The CRISPR/Cas-based base editing system of claim 2, wherein the fusion protein comprises a polypeptide selected from SEQ ID NOs: 27-34 and/or is encoded by a polynucleotide comprising a sequence selected from SEQ ID NOs: 35-42.

4. The CRISPR/Cas-based base editing system of any one of claims 1-3, wherein altering the RNA splice site encoded in the genomic DNA results in exclusion or inclusion of at least one exon sequence in an RNA transcript.

5. A CRISPR/Cas-based base editing system for restoring dystrophin function in a subject, the CRISPR/Cas-based base editing system comprising a fusion protein and at least one guide RNA (gRNA), wherein the fusion protein comprises a Cas protein and a base-editing domain,
wherein the at least one gRNA targets a sequence comprising at least one of SEQ ID NOs: 21-23 or 43 or a complement or a fragment thereof and/or the gRNA comprises a sequence selected from SEQ ID NOs: 24-26 or 44 or a complement or a fragment thereof.

6. A CRISPR/Cas-based base editing system for restoring dystrophin function in a subject, the CRISPR/Cas-based base editing system comprising a fusion protein and at least one guide RNA (gRNA), wherein the fusion protein comprises a Cas protein and a base-editing domain, and
wherein base-editing domain comprises a polypeptide selected from SEQ ID NOs: 45-52 and/or is encoded by a polynucleotide comprising a sequence selected from SEQ ID NOs: 53-80.

7. The CRISPR/Cas-based base editing system of claim 6, wherein the fusion protein comprises a polypeptide selected from SEQ ID NOs: 27-34 and/or is encoded by a polynucleotide comprising a sequence selected from SEQ ID NOs: 35-42.

8. The CRISPR/Cas-based base editing system of any one of claims 5-7, wherein the subject has a mutated dystrophin gene, and wherein the at least one guide RNA (gRNA) targets an RNA splice site in the mutated dystrophin gene of the subject.

9. The CRISPR/Cas-based base editing system of claim 8, wherein administration of the CRISPR/Cas-based base editing system to the subject results in at least one exon sequence being excluded or included in an RNA transcript of the dystrophin gene of the subject and the reading frame of dystrophin gene in the subject being restored.

10. The CRISPR/Cas-based base editing system any one of claims 1-9, wherein the Cas protein comprises a Cas9, and wherein the Cas9 comprises at least one amino acid mutation which eliminates the nuclease activity of Cas9.

11. The CRISPR/Cas-based base editing system of claim 10, wherein the at least one amino acid mutation is at least one of D10A, H840A, or a combination thereof, in the amino acid sequence corresponding to SEQ ID NO: 2 or 3.

12. The CRISPR/Cas-based base editing system of any one of claims 1-11, wherein the Cas protein is a Streptococcus pyogenes Cas9 protein or a Staphylococcus aureus Cas9 protein.

13. The CRISPR/Cas-based base editing system of any one of claims 1-12, wherein the Cas protein comprises an amino acid sequence of SEQ ID NO: 4 or 5.

14. The CRISPR/Cas-based base editing system of any one of claims 1-13, wherein the base-editing domain further comprises (i) a cytidine deaminase domain and (ii) at least one uracil glycosylase inhibitor (UGI) domain.

15. The CRISPR/Cas-based base editing system of claim 14, wherein the cytidine deaminase domain comprises an apolipoprotein B mRNA-editing enzyme, catalytic polypeptide-like (APOBEC) deaminase.

16. The CRISPR/Cas-based base editing system of claim 14 or 15, wherein the cytidine deaminase domain comprises an APOBEC 1 deaminase.

17. The CRISPR/Cas-based base editing system of claim 16, wherein the cytidine deaminase domain comprises a rat APOBEC 1 deaminase.

18. The CRISPR/Cas-based base editing system of any one of claims 14-17, wherein the at least one UGI domain comprises a domain capable of inhibiting UDG activity.

19. The CRISPR/Cas-based base editing system of claim 18, wherein the at least one UGI domain comprises the amino acid sequence of SEQ ID NO: 20 or an amino acid sequence encoded by the polynucleotide sequence of SEQ ID NO: 6 or SEQ ID NO: 18.

20. The CRISPR/Cas-based base editing system of any one of claims 14-19, wherein the base-editing domain comprises one UGI domain or two UGI domains.

21. The CRISPR/Cas-based base editing system of any one of claims 1-20, wherein the fusion protein comprises the structure: NH₂-[ABE]-[Cas protein]-COOH, and wherein each instance of “-” comprises an optional linker.

22. The CRISPR/Cas-based base editing system of any one of claims 1-20, wherein the fusion protein comprises the structure: NH₂-[Cas protein]-[ABE]-COOH, and wherein each instance of “-” comprises an optional linker.

23. The CRISPR/Cas-based base editing system of any one of claims 1-22, wherein the fusion protein further comprises a nuclear localization sequence (NLS).

24. An isolated polynucleotide encoding the CRISPR/Cas-based base editing system of any one of claims 1-23.

25. The isolated polynucleotide of claim 24, wherein the polynucleotide comprises a first polynucleotide encoding the fusion protein and a second polynucleotide encoding the gRNA.

26. A vector comprising the isolated polynucleotide of claim 24 or 25.

27. The vector of claim 26, wherein the vector comprises a heterologous promoter driving expression of the isolated polynucleotide.

28. A cell comprising the isolated polynucleotide of claim 24 or 25 or the vector of claim 26 or 27.

29. A composition for restoring dystrophin function in a cell having a mutant dystrophin gene, the composition comprising the CRISPR/Cas-based base editing system of any one of claims 1-23.

30. A kit comprising the CRISPR/Cas-based base editing system of any one of claims 1-23, the isolated polynucleotide of claim 24 or 25, the vector of claim 26 or 27, the cell of claim 28, or the composition of claim 29.

31. A method for restoring dystrophin function in a cell or a subject having a mutant dystrophin gene, the method comprising contacting the cell or the subject with the CRISPR/Cas-based base editing system of any one of claims 1-23.

32. The method of claim 31, wherein an “AG” splice acceptor in exon 45 of the mutant dystrophin gene is converted to an “GG” sequence and the dystrophin function is restored by exon 45 skipping.

33. The method of claim 31 or 32, wherein the subject is suffering from Duchenne Muscular Dystrophy.


SEQUENCE LISTING (74 sequences)

SEQ ID NO: 1 - DNA, 20 bp, Artificial Sequence
  Synthetic
gttcctgtaagataccaaaa

SEQ ID NO: 2 - protein, 1368 aa, Streptococcus pyogenes [sequence omitted]

SEQ ID NO: 3 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 4 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 6 - DNA, 249 bp, Artificial Sequence
  Synthetic
actaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctg

SEQ ID NO: 7 - DNA, 8961 bp, Artificial Sequence
  Synthetic
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 8 - DNA, 8961 bp, Artificial Sequence
  Synthetic
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtcagcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctaaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctaggatgcctaatgagtgagctaactcacattaattgcgttgcgctcactgcccgctttccagtcgggaaacctgtcgtgccagctgcattaatgaatcggccaacgcgcgggaagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 9 - DNA, 20 bp, Artificial Sequence
  Synthetic
cgcctgcaggtaaaagcata

SEQ ID NO: 10 - DNA, 3 bp, Artificial Sequence
  Synthetic
  misc_feature (1)..(1): n is a, c, g, or t
ngg

SEQ ID NO: 11 - DNA, 6 bp, Artificial Sequence
  Synthetic
  misc_feature (1)..(3): n is a, c, g, or t
  r (4)..(5): r is a, or g
nnnrrt

SEQ ID NO: 12 - DNA, 5 bp, Artificial Sequence
  Synthetic
  misc_feature (1)..(2): n is a, c, g, or t
  r (4)..(5): r is a, or g
nngrr

SEQ ID NO: 13 - DNA, 6 bp, Artificial Sequence
  Synthetic
  misc_feature (1)..(2): n is a, c, g, or t
  r (4)..(5): r is a, or g
  misc_feature (6)..(6): n is a, c, g, or t
nngrrn

SEQ ID NO: 14 - DNA, 6 bp, Artificial Sequence
  Synthetic
  misc_feature (1)..(2): n is a, c, g, or t
  r (4)..(5): r is a, or g
nngrrt

SEQ ID NO: 15 - DNA, 6 bp, Artificial Sequence
  Synthetic
  misc_feature (1)..(2): n is a, c, g, or t
  r (4)..(5): r is a, or g
  v (6)..(6): v is a, c, or g
nngrrv

SEQ ID NO: 16 - DNA, 20 bp, Artificial Sequence
  Synthetic
ctacaacaaagctcaggtcg

SEQ ID NO: 17 - DNA, 23 bp, Artificial Sequence
  Synthetic
ttctcaggtaaagctctggaaac

SEQ ID NO: 18 - DNA, 249 bp, Artificial Sequence
  Synthetic
accaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctg

SEQ ID NO: 19 - DNA, 3 bp, Artificial Sequence
  Synthetic
  misc_feature (1)..(1): n is a, c, g, or t
nga

SEQ ID NO: 20 - protein, 83 aa, Artificial Sequence
  Synthetic
TNLSDIIEKETGKQLVIQESILMLPEEVEEVIGNKPESDILVHTAYDESTDENVMLLTSDAPEYKPWALVIQDSNGENKIKML

SEQ ID NO: 21 - DNA, 20 bp, Artificial Sequence
  Synthetic
tggtatcttacaggaactcc

SEQ ID NO: 22 - DNA, 20 bp, Artificial Sequence
  Synthetic
atcttacaggaactccagga

SEQ ID NO: 23 - DNA, 20 bp, Artificial Sequence
  Synthetic
caggaactccaggatggcat

SEQ ID NO: 24 - RNA, 20 nt, Artificial Sequence
  Synthetic
ugguaucuuacaggaacucc

SEQ ID NO: 25 - RNA, 20 nt, Artificial Sequence
  Synthetic
aucuuacaggaacuccagga

SEQ ID NO: 26 - RNA, 20 nt, Artificial Sequence
  Synthetic
caggaacuccaggauggcau

SEQ ID NO: 27 - protein, 1775 aa, Artificial Sequence
  Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 28 - protein, 1775 aa, Artificial Sequence
  Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 29 - protein, 1803 aa, Artificial Sequence
  Synthetic
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 30 - protein, 1605 aa, Artificial Sequence
  Synthetic
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 31 - protein, 1588 aa, Artificial Sequence
  Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 32 - protein, 1588 aa, Artificial Sequence
  Synthetic
MSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 33 - protein, 1588 aa, Artificial Sequence
  Synthetic
MSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 34 - protein, 1588 aa, Artificial Sequence
  Synthetic
MSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGADKRTADGSEFESPKKKRKV

SEQ ID NO: 35 - DNA, 5328 bp, Artificial Sequence
  Synthetic
atgtccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttctggtggttcttctggtggttcttccgaagtcgagttttcccatgagtactggatgagacacgcattgactctcgcaaagagggctctcgatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtaacgcgctgttgtgttacttttttcgcatgcccaggcaggtctttaacgcccagaaaaaagcacaatcctctactgactctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctcccaagaagaagaggaaagtctaa

SEQ ID NO: 36 - DNA, 5328 bp, Artificial Sequence
  Synthetic
atgtccgaagtcgagttttcccatgagtactggatgagacacgcattgactctcgcaaagagggcttgggatgaacgcgaggtgcccgtgggggcagtactcgtgcataacaatcgcgtaatcggcgaaggttggaataggccgatcggacgccacgaccccactgcacatgcggaaatcatggcccttcgacagggagggcttgtgatgcagaattatcgacttatcgatgcgacgctgtacgtcacgcttgaaccttgcgtaatgtgcgcgggagctatgattcactcccgcattggacgagttgtattcggtgcccgcgacgccaagacgggtgccgcaggttcactgatggacgtgctgcatcacccaggcatgaaccaccgggtagaaatcacagaaggcatattggcggacgaatgtgcggcgctgttgtccgacttttttcgcatgcggaggcaggagatcaaggcccagaaaaaagcacaatcctctactgactctggtggttcttctggtggttctagcggcagcgagactcccgggacctcagagtccgccacacccgaaagttctggtggttcttctggtggttcttccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgactctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctcccaagaagaagaggaaagtctaa

SEQ ID NO: 37 - DNA, 5412 bp, Artificial Sequence
  Synthetic
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 38 - DNA, 4818 bp, Artificial Sequence
  Synthetic
atgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtctaa

SEQ ID NO: 39 - DNA, 4767 bp, Artificial Sequence
  Synthetic
atgtccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgagggagctgataagcgcaccgccgatggttccgagttcgaaagccccaagaagaagaggaaagtctaa

SEQ ID NO: 40 - DNA, 4767 bp, Artificial Sequence
  Synthetic
atgtccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgagggagctgataagcgcaccgccgatggttccgagttcgaaagccccaagaagaagaggaaagtctaa

SEQ ID NO: 41 - DNA, 4767 bp, Artificial Sequence
  Synthetic
atgtccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcgtgtctttaacgcccagaaaaaagcacaatcctctactgactctggtggttcttctggtggttctagcggcagcgagactcccgggacctcagagtccgccacacccgaaagttctggtggttcttctggtggttctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgagggagctgataagcgcaccgccgatggttccgagttcgaaagccccaagaagaagaggaaagtctaa

SEQ ID NO: 42 - DNA, 4767 bp, Artificial Sequence
  Synthetic
atgtccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttctgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacgagggagctgataagcgcaccgccgatggttccgagttcgaaagccccaagaagaagaggaaagtctaa

SEQ ID NO: 43 - DNA, 19 bp, Artificial Sequence
  Synthetic
gttcctgtaagataccaaa

SEQ ID NO: 44 - RNA, 19 nt, Artificial Sequence
  Synthetic
guuccuguaagauaccaaa

SEQ ID NO: 45 - protein, 166 aa, Artificial Sequence
  Synthetic
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 46 - protein, 166 aa, Artificial Sequence
  Synthetic
SEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 47 - protein, 166 aa, Artificial Sequence
  Synthetic
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 48 - protein, 166 aa, Artificial Sequence
  Synthetic
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 49 - protein, 166 aa, Artificial Sequence
  Synthetic
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 50 - protein, 166 aa, Artificial Sequence
  Synthetic
SEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 51 - protein, 166 aa, Artificial Sequence
  Synthetic
SEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTD

SEQ ID NO: 52 - protein, 166 aa, Artificial Sequence
  Synthetic
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 53 - DNA, 498 bp, Artificial Sequence
  Synthetic
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgac

SEQ ID NO: 54 - DNA, 498 bp, Artificial Sequence
  Synthetic
tccgaagtcgagttttcccatgagtactggatgagacacgcattgactctcgcaaagagggctctcgatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtaacgcgctgttgtgttacttttttcgcatgcccaggcaggtctttaacgcccagaaaaaagcacaatcctctactgac

SEQ ID NO: 55 - DNA, 498 bp, Artificial Sequence
  Synthetic
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcaggtctttaacgcccagaaaaaagcacaatcctctactgac

SEQ ID NO: 56 - DNA, 498 bp, Artificial Sequence
  Synthetic
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 57 - DNA, 594 bp, Artificial Sequence
  Synthetic
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgactctggtggttcttctggtggttctagcggcagcgagactcccgggacctcagagtccgccacacccgaaagttctggtggttcttctggtggttct

SEQ ID NO: 58 - DNA, 498 bp, Artificial Sequence
  Synthetic
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtacgtcacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgac

SEQ ID NO: 59 - DNA, 498 bp, Artificial Sequence
  Synthetic
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgacttatcgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcattacccaggcatgaaccaccgggtagaaatcacagaaggcatattggcggacgaatgtgcggcgctgttgtgttacttttttcgcatgcccaggcgtgtctttaacgcccagaaaaaagcacaatcctctactgac

SEQ ID NO: 60 - DNA, 498 bp, Artificial Sequence
  Synthetic
tccgaagtcgagttttcccatgagtactggatgagacacgcattgactctcgcaaagagggctcgagatgaacgcgaggtgcccgtgggggcagtactcgtgctcaacaatcgcgtaatcggcgaaggttggaatagggcaatcggactccacgaccccactgcacatgcggaaatcatggcccttcgacagggagggcttgtgatgcagaattatcgactttatgatgcgacgctgtactcgacgtttgaaccttgcgtaatgtgcgcgggagctatgattcactcccgcattggacgagttgtattcggtgttcgcaacgccaagacgggtgccgcaggttcactgatggacgtgctgcatcatccaggcatgaaccaccgggtagaaatcacagaaggcatattggcggacgaatgtgcggcgctgttgtgtcgtttttttcgcatgcccaggcgggtctttaacgcccagaaaaaagcacaatcctctactgac

SEQ ID NO: 61 - protein, 32 aa, Artificial Sequence
  Synthetic
SGGSSGGSSGSETPGTSESATPESSGGSSGGS

SEQ ID NO: 62 - protein, 4 aa, Artificial Sequence
  Synthetic
SGGS

SEQ ID NO: 63 - DNA, 96 bp, Artificial Sequence
  Synthetic
tctggtggttcttctggtggttctagcggcagcgagactcccgggacctcagagtccgccacacccgaaagttctggtggttcttctggtggttct

SEQ ID NO: 64 - DNA, 12 bp, Artificial Sequence
  Synthetic
tctggtggttct

SEQ ID NO: 65 - protein, 7 aa, Artificial Sequence
  Synthetic
PKKKRKV

SEQ ID NO: 66 - protein, 17 aa, Artificial Sequence
  Synthetic
KRTADGSEFEPKKKRKV

SEQ ID NO: 67 - protein, 18 aa, Artificial Sequence
  Synthetic
KRTADGSEFESPKKKRKV

SEQ ID NO: 68 - protein, 22 aa, Artificial Sequence
  Synthetic
EGADKRTADGSEFESPKKKRKV

SEQ ID NO: 69 - DNA, 21 bp, Artificial Sequence
  Synthetic
cccaagaagaagaggaaagtc

SEQ ID NO: 70 - DNA, 51 bp, Artificial Sequence
  Synthetic
aaaagaaccgccgacggcagcgaattcgagcccaagaagaagaggaaagtc

SEQ ID NO: 71 - DNA, 54 bp, Artificial Sequence
  Synthetic
aaacggacagccgacggaagcgagttcgagtcaccaaagaagaagcggaaagtc

SEQ ID NO: 72 - DNA, 66 bp, Artificial Sequence
  Synthetic
gagggagctgataagcgcaccgccgatggttccgagttcgaaagccccaagaagaagaggaaagtc

SEQ ID NO: 73 - DNA, 86 bp, Artificial Sequence
  Synthetic
gtttaagagctatgctggaaacagcatagcaagtttaaataaggctagtccgttatcaacttgaaaaagtggcaccgagtcggtgc

SEQ ID NO: 74 - RNA, 86 nt, Artificial Sequence
  Synthetic
guuuaagagcuaugcuggaaacagcauagcaaguuuaaauaaggcuaguccguuaucaacuugaaaaaguggcaccgagucggugc